(19) United States
(12) Patent Application Publication
(10) Pub. No.: US 2026/0022162 A1
(43) Pub. Date: Jan. 22, 2026

(54) ANTI-HIV VACCINE ANTIBODIES WITH REDUCED POLYREACTIVITY

(71) Applicants: California Institute of Technology, Pasadena, CA (US); The Rockefeller University, New York, NY (US)

(72) Inventors: Stuart A. Sievers, Encino, CA (US); Jennifer Keeffe, Pasadena, CA (US); Michel C. Nussenzweig, New York, NY (US); Pamela J. Bjorkman, La Canada, CA (US)

(21) Appl. No.: 19/343,891

(22) Filed: Sep. 29, 2025

Related U.S. Application Data
(62) Division of application No. 17/625,247, filed on Jan. 6, 2022, now Pat. No. 12,459,990.
(60) Provisional application No. 62/871,393, filed on Jul. 8, 2019.

(51) Int. Cl. C07K 16/10; A61K 39/42; A61K 45/06; A61P 31/18

(52) U.S. Cl. CPC C07K 16/1045; A61K 39/42; A61K 45/06; A61P 31/18; C07K 2317/31; C07K 2317/565

(57) ABSTRACT

This disclosure provides novel broadly neutralizing anti-HIV antibodies and antigen-binding fragments thereof. The disclosed anti-HIV antibodies exhibited improved biophysical properties, e.g., reduced polyreactivity, prolonged half-life, while retaining broad and potent neutralization activity. The anti-HIV bNAb variants as disclosed constitute a novel therapeutic strategy for treating and/or preventing HIV infection.

### CROSS-REFERENCE TO RELATED APPLICATIONS

The present application is a divisional application of U.S. patent application Ser. No. 17/625,247, filed on Jan. 6, 2022, which is a National Stage Application under 35 U.S.C. § 371 of International Application No. PCT/US20/41138 filed on Jul. 8, 2020, which claims priority under 35 U.S.C. § 119 (c) to U.S. Provisional Patent Application No. 62/871,393, filed on Jul. 8, 2019, the disclosures of all of which are hereby incorporated by reference in their entireties.

### STATEMENT REGARDING FEDERALLY SPONSORED RESEARCH

This invention was made with government support under AI100148 and AI129784 awarded by the National Institutes of Health. The government has certain rights in the invention.

### REFERENCE TO AN ELECTRONIC SEQUENCE LISTING

The contents of the electronic sequence listing (070413.20853_SeqList.xml; Size: 169,693 bytes; and Date of Creation: Aug. 16, 2025) is herein incorporated by reference in its entirety.

### FIELD OF THE INVENTION

This invention relates to broad and potent antibodies against Human Immunodeficiency Virus (“HIV”).

### BACKGROUND OF THE INVENTION

HIV causes acquired immunodeficiency syndrome (AIDS), a condition in humans characterized by clinical features including wasting syndromes, central nervous system degeneration and profound immunosuppression that results in life-threatening opportunistic infections and malignancies. Since its discovery in 1981, HIV type 1 (HIV-1) has led to the death of at least 25 million people worldwide. It is predicted that 20-60 million people will become infected over the next two decades, even if there is a 2.5% annual decrease in HIV infections. There is a need for therapeutic agents and methods for treatment or inhibition of HIV infection.

Broadly neutralizing antibodies (bNAbs) against HIV are being developed as potential therapeutics for treatment and/or prevention of HIV infection. Many bNAbs have been isolated from human donors and characterized. For some of these bNAbs, modifications have been engineered to improve their neutralization breadth and/or potency. One such engineered bNAb is NIH45-46 with the heavy chain mutation G54W (NIH45-46 G54W). Although NIH45-46 G54W has a desirable neutralization profile, it has biophysical properties, such as polyreactivity/non-specific binding, a short in vivo half-life, and a propensity to aggregate, which hinder its development as a human therapeutic. Although there are various known approaches to improving the biophysical properties of an antibody, it can be difficult to improve these properties without sacrificing much of the antibody's neutralizing activity.

### SUMMARY OF THE INVENTION

In one aspect, this disclosure provides novel isolated broadly neutralizing anti-HIV antibodies, or antigen-binding portions thereof, with improved biophysical properties, such as reduced polyreactivity. The isolated anti-HIV antibody, or antigen-binding portion thereof, comprises: a heavy chain having a heavy chain amino acid sequence that is at least 75% (e.g., 80%, 85%, 90%, 95%, 96%, 97%, 98%, 99% or more) identical to SEQ ID NO: 1, wherein the heavy chain amino acid sequence comprises one or more heavy chain substitutions at one or more residues selected from the group consisting of S5, S21, G54, G55, V57, T68, T70, V73, S75, F79, S82, D85, V89, Y97, and P112; or a light chain having a light chain amino acid sequence that is at least 75% (e.g., 80%, 85%, 90%, 95%, 96%, 97%, 98%, 99% or more) identical to SEQ ID NO: 2, wherein the light chain amino acid sequence comprises one or more light chain substitutions at one or more residues selected from the group consisting of T5, S7, T10, S12, S14, T18, 120, 20) S22, R24, Q27, S28, S30, R59, S61, S63, W65, D68, N70, S72, S74, and S78, wherein residue numbering is set forth with the Kabat numbering.

In some embodiments, the isolated anti-HIV antibody, or antigen-binding portion thereof, comprises: the heavy chain having the heavy chain polypeptide sequence that is at least 75% (e.g., 80%, 85%, 90%, 95%, 96%, 97%, 98%, 99% or more) identical to SEQ ID NO: 1, wherein the heavy chain amino acid sequence comprises the one or more heavy chain substitutions at the one or more residues selected from the group consisting of S5, S21, G54, G55, V57, T68, T70, V73, S75, F79, S82, D85, V89, Y97, and P112; and the light chain having the light chain amino acid sequence that is at least 75% (e.g., 80%, 85%, 90%, 95%, 96%, 97%, 98%, 99% or more) identical to SEQ ID NO: 2, wherein the light chain amino acid sequence comprises one or more light chain substitutions at one or more residues selected from the group consisting of T5, S7, T10, S12, S14, T18, 120, S22, R24, Q27, S28, S30, R59, S61, S63, W65, D68, N70, S72, S74, and S78.

In some embodiments, the one or more heavy chain substitutions comprise: a substitution of S5D or S5E, or a conservative substitution of Asp or Glu at residue S5; a substitution of S21D or S21E, or a conservative substitution of Asp or Glu at residue S21; a substitution of G54W or a conservative substitution of Trp at residue G54; a substitution of G55D or G55E, or a conservative substitution of Asp or Glu at residue G55; a substitution of V57D or V57E, or a conservative substitution of Asp or Glu at residue V57; a substitution of T68D or T68E, or a conservative substitution of Asp or Glu at residue T68; a substitution of T70D or T70E, or a conservative substitution of Asp or Glu at residue T70; a substitution of V73D or V73E, or a conservative substitution of Asp or Glu at residue V73; a substitution of S75D or S75E, or a conservative substitution of Asp or Glu at residue S75; a substitution of F79D, F79E, F79Y or F79H, or a conservative substitution of Asp, Glu, Tyr or His at residue F79; a substitution of S82D or S82E, or a conservative substitution of Asp or Glu at residue S82; a substitution of D85E, or a conservative substitution of Asp at residue D85; a substitution of V89D or V89E, or a conservative substitution of Asp or Glu at residue V89; a substitution of Y97D or Y97E, or a conservative substitution of Asp or Glu at residue Y97; a substitution of P112D or P112E, or a conservative substitution of Asp or Glu at residue P112; or a combination thereof.

In some embodiments, the one or more light chain substitutions comprises: a substitution of T5D or T5E, or a conservative substitution of Asp or Glu at residue T5; a substitution of S7D or STE, or a conservative substitution of Asp or Glu at residue S7; a substitution of T10D or T10E, or a conservative substitution of Asp or Glu at residue T10; a substitution of S12D or S12E, or a conservative substitution of Asp or Glu at residue S12; a substitution of S14D or S14E, or a conservative substitution of Asp or Glu at residue S14; a substitution of T18D or T18E, or a conservative substitution of Asp or Glu at residue T18; a substitution of 120D or 120E, or a conservative substitution of Asp or Glu at residue 120; a substitution of S22D or S22E, or a conservative substitution of Asp or Glu at residue S22; a substitution of R24D or R24E, or a conservative substitution of Asp or Glu at residue R24; a substitution of Q27D or Q27E, or a conservative substitution of Asp or Glu at residue Q27; a substitution of S28Y or S28H, or a conservative substitution of Tyr or His at residue S28; a substitution of S30D or S30E, or a conservative substitution of Asp or Glu at residue S30; a substitution of R59D or R59E, or a conservative substitution of Asp or Glu at residue R59; a substitution of S61D or S61E, or a conservative substitution of Asp or Glu at residue S61; a substitution of S63D or S63E, or a conservative substitution of Asp or Glu at residue S63; a substitution of W65D, W65E, W65Y or W65H, or a conservative substitution of Asp, Glu, Tyr or His at residue W65; a substitution of D68E, or a conservative substitution of Asp at residue D68; a substitution of N70D or N70E, or a conservative substitution of Asp or Glu at residue N70; a substitution of S72D or S72E, or a conservative substitution of Asp or Glu at residue S72; a substitution of S74D or S74E, or a conservative substitution of Asp or Glu at residue S74; a substitution of S78D or S78E, or a conservative substitution of Asp or Glu at residue S78; or a combination thereof.

In some embodiments, the one or more heavy chain substitutions further comprise substitutions of M428L and N434S.

In some embodiments, the light chain amino acid sequence comprises the one or more light chain substitutions of: (i) Q27E, S28H, and S30D; (ii) Q27E, S28H, S30D, and S74T; (iii) Q27E, S28H, S30D, S74T, M428L, and N434S; (iv) Q27E, S28Y, and S30D; (v) Q27D and S28H; (vi) S28Y; (vii) T5D, T10D, S12D, S14D, 120D, and S22D; or (viii) S61D, S63D, W65D, N70D, S72D, and S74D.

In some embodiments, the heavy chain amino acid sequence comprises the one or more heavy chain substitutions comprising G54W and T68D, and the light chain amino acid sequence comprises the one or more light chain substitutions of: (a) Q27E, S28H, S30D, and S74T; (b) Q27E, S28H, S30D, S74T, M428L, and N434S; (c) Q27E, S28Y, and S30D; (d) Q27D and S28H; or (e) S28Y.

In some embodiments, the heavy chain amino acid sequence comprises the one or more heavy chain substitutions comprising G54W; and the light chain amino acid sequence comprises the one or more light chain substitutions of: (a) Q27E, S28H, and S30D; (b) Q27E, S28Y, and S30D; or (c) Q27D and S28H.

In some embodiments, the heavy chain amino acid sequence comprises the amino acid sequence of SEQ ID NO: 3-35. In some embodiments, the light chain amino acid sequence comprises an amino acid sequence of SEQ ID NOs: 2 and 36-80.

In some embodiments, the isolated anti-HIV antibody comprises a CDRH 1, a CDRH 2, a CDRH 3, a CDRL 1, a CDRL 2, and a CDRL 3, comprising respective sequences set forth in a CDR sequence set of: SEQ ID NOs: 81, 84, 87, 90, 93, and 96; SEQ ID NOs: 81, 84, 87, 99, 93, and 96; SEQ ID NOs: 81, 84, 87, 100, 93, and 96; SEQ ID NOs: 81, 84, 87, 101, 93, and 96; or SEQ ID NOs: 81, 84, 87, 102, 93, and 96; (set forth with the Kabat numbering); SEQ ID NOs: 82, 85, 88, 91, 94, and 97 (set forth with the IMGT numbering); or SEQ ID NOs: 83, 86, 89, 92, 95, and 98 (set forth with the Chothia numbering).

In some embodiments, the heavy chain amino acid sequence and the light chain amino acid sequence comprise the respective amino acid sequences set forth in a sequence set selected from the group consisting of SEQ ID NOs: 3 and 36; 3 and 38; 3 and 39; 4 and 2; 4 and 38; 4 and 39; 4 and 41; and 34 and 37.

In some embodiments, the isolated anti-HIV antibody is a bispecific antibody comprising a first antigen binding arm binding to a first antigen and a second antigen binding arm binding to a second antigen, wherein the first antigen and the second antigen are different, and wherein the first antigen binding arm comprising the heavy chain amino acid sequence and the light chain amino acid sequence as specified above. In some embodiments, the second antigen is selected from the group consisting of CD3, FcγRI (CD64), FcγRII (CD32), FcγRIII (CD 16); CD89, CCR5, CD4, CD8, CD28, CD137, CTLA-4, gp41, killer cell immunoglobulin like receptor, three Ig domains and long cytoplasmic tail 1 (KIR3DL1), killer cell immunoglobulin like receptor, three Ig domains and long cytoplasmic tail 1 (KIR3DL1), killer cell immunoglobulin like receptor, two Ig domains and long cytoplasmic tail 1 (KIR2DL1), killer cell immunoglobulin like receptor, two Ig domains and long cytoplasmic tail 2 (KIR2DL2), killer cell immunoglobulin like receptor, two Ig domains and long cytoplasmic tail 3 (KIR2DL3), killer cell lectin like receptor C1 (KLRC1), killer cell lectin like receptor C2 (KLRC2), killer cell lectin like receptor C3 (KLRC3), killer cell lectin like receptor C4 (KLRC4), killer cell lectin like receptor D1 (KLRD1), killer cell lectin like receptor K1 (KLRK1), natural cytotoxicity triggering receptor 3 (NCR3 or NKp30), natural cytotoxicity triggering receptor 2 (NCR2 or NK-p44), natural cytotoxicity triggering receptor 1 (NCR1 or NK-p46), CD226 (DNAM-1), cytotoxic and regulatory T cell molecule (CRTAM or CD355), signaling lymphocytic activation molecule family member 1 (SLAMF1), CD48 (SLAMF2), lymphocyte antigen 9 (LY9 or SLAMF3), CD244 (2B4 or SLAMF4), CD84 (SLAMF5), SLAM family member 6 (SLAMF6 or NTB-A), SLAM family member 7 (SLAMF7 or CRACC), CD27 (TNFRSF7), semaphorin 4D (SEMA4D or CD 100), CD 160 (NK1), an immune checkpoint protein (e.g., PD-1, PD-L1, CTLA-4), and a second epitope of gp120.

Also provided in this disclosure are (i) an isolated nucleic acid comprising a sequence encoding a CDR, a heavy chain variable region, or a light chain variable region of the anti-HIV antibody, or antigen binding portion thereof, as described above; (ii) a vector comprising the nucleic acid described above; and (iii) a cultured cell comprising the vector described above.

Also within the scope of this disclosure is a method for making an anti-HIV antibody or a fragment thereof. The method comprises: (a) obtaining the cultured cell described above; (b) culturing the cell in a medium under conditions permitting expression of a polypeptide encoded by the vector and assembling of an antibody or fragment thereof; and (c) purifying the antibody or fragment from the cultured cell or the medium of the cell.

In another aspect, this disclosure also provides a pharmaceutical composition comprising (i) at least one anti-HIV antibody, or antigen binding portion thereof, the nucleic acid, or the vector, as described above; and (ii) a pharmaceutically acceptable carrier.

In some embodiments, the pharmaceutical composition further comprises a second therapeutic agent. In some embodiments, the second therapeutic agent comprises an antiviral agent or one or more additional antibodies. In some embodiments, the one or more additional antibodies comprise a second anti-HIV antibody (e.g., an isolated anti-HIV bNAb as disclosed) or antigen binding portion thereof, or a third antibody binding to a third antigen. In some embodiments, the third antigen is selected from the group consisting of CD3, FcγRI (CD64), FcγRII (CD32), FcγRIII 20) (CD 16); CD89, CCR5, CD4, CD8, CD28, CD137, CTLA-4, gp41, killer cell immunoglobulin like receptor, three Ig domains and long cytoplasmic tail 1 (KIR3DL1), killer cell immunoglobulin like receptor, three Ig domains and long cytoplasmic tail 1 (KIR3DL1), killer cell immunoglobulin like receptor, two Ig domains and long cytoplasmic tail 1 (KIR2DL1), killer cell immunoglobulin like receptor, two Ig domains and long cytoplasmic tail 2 (KIR2DL2), killer cell immunoglobulin like receptor, two Ig domains and long cytoplasmic tail 3 (KIR2DL3), killer cell lectin like receptor C1 (KLRC1), killer cell lectin like receptor C2 (KLRC2), killer cell lectin like receptor C3 (KLRC3), killer cell lectin like receptor C4 (KLRC4), killer cell lectin like receptor D1 (KLRD1), killer cell lectin like receptor K1 (KLRK1), natural cytotoxicity triggering receptor 3 (NCR3 or NKp30), natural cytotoxicity triggering receptor 2 (NCR2 or NK-p44), natural cytotoxicity triggering receptor 1 (NCR1 or NK-p46), CD226 (DNAM-1), cytotoxic and regulatory T cell molecule (CRTAM or CD355), signaling lymphocytic activation molecule family member 1 (SLAMF1), CD48 (SLAMF2), lymphocyte antigen 9 (LY9 or SLAMF3), CD244 (2B4 or SLAMF4), CD84 (SLAMF5), SLAM family member 6 (SLAMF6 or NTB-A), SLAM family member 7 (SLAMF7 or CRACC), CD27 (TNFRSF7), semaphorin 4D (SEMA4D or CD 100), CD 160 (NK1), an immune checkpoint protein (e.g., PD-1, PD-L1, CTLA-4), and a second epitope of gp120.

In some embodiments, the antiviral agent is selected from the group consisting of a non-nucleoside reverse transcriptase inhibitor, a protease inhibitor, an entry or fusion inhibitor, and an integrase inhibitor.

In yet another aspect, this disclosure provides a method of preventing or treating an HIV infection or an HIV-related disease. The method comprises (a) identifying a patient in need of such prevention or treatment, and (b) administering to the patient a first therapeutic agent comprising a therapeutically effective amount of at least one anti-HIV antibody, or antigen-binding portion thereof, as described above.

In some embodiments, the method further comprises administering a second therapeutic agent. In some embodiments, the second therapeutic agent comprises an antiviral agent or one or more additional antibodies. In some embodiments, wherein the one or more additional antibodies comprise a second anti-HIV antibody (e.g., an isolated anti-HIV bNAb as disclosed) or antigen binding portion thereof, or a third antibody binding to a third antigen. In some embodiments, the third antigen is selected from the group consisting of CD3, FcγRI (CD64), FcγRII (CD32), FcγRIII (CD 16); CD89, CCR5, CD4, CD8, CD28, CD137, CTLA-4, gp41, killer cell immunoglobulin like receptor, three Ig domains and long cytoplasmic tail 1 (KIR3DL1), killer cell immunoglobulin like receptor, three Ig domains and long cytoplasmic tail 1 (KIR3DL1), killer cell immunoglobulin like receptor, two Ig domains and long cytoplasmic tail 1 (KIR2DL1), killer cell immunoglobulin like receptor, two Ig domains and long cytoplasmic tail 2 (KIR2DL2), killer cell immunoglobulin like receptor, two Ig domains and long cytoplasmic tail 3 (KIR2DL3), killer cell lectin like receptor C1 (KLRC1), killer cell lectin like receptor C2 (KLRC2), killer cell lectin like receptor C3 (KLRC3), killer cell lectin like receptor C4 (KLRC4), killer cell lectin like receptor D1 (KLRD1), killer cell lectin like receptor K1 (KLRK1), natural cytotoxicity triggering receptor 3 (NCR3 or NKp30), natural cytotoxicity triggering receptor 2 (NCR2 or NK-p44), natural cytotoxicity triggering receptor 1 (NCR1 or NK-p46), CD226 (DNAM-1), cytotoxic and regulatory T cell molecule (CRTAM or CD355), signaling lymphocytic activation molecule family member 1 (SLAMF1), CD48 (SLAMF2), lymphocyte antigen 9 (LY9 or SLAMF3), CD244 (2B4 or SLAMF4), CD84 (SLAMF5), SLAM family member 6 (SLAMF6 or NTB-A), SLAM family member 7 (SLAMF7 or CRACC), CD27 (TNFRSF7), semaphorin 4D (SEMA4D or CD 100), CD 160 (NK1), an immune checkpoint protein (e.g., PD-1, PD-L1, CTLA-4), and a second epitope of gp120.

In some embodiments, the first therapeutic agent or the second therapeutic agent is administered to the patient intratumorally, intravenously, subcutaneously, intraosscously, orally, transdermally, or sublingually. In some embodiments, the first therapeutic agent is administered to the patient before, after, or concurrently with the second therapeutic agent.

In another aspect, this disclosure additionally provides a kit comprising a pharmaceutically acceptable dose unit of a pharmaceutically effective amount of at least one isolated anti-HIV antibody, or antigen-binding portion thereof, as described above.

In some embodiments, the kit further comprises a pharmaceutically acceptable dose unit of a pharmaceutically effective amount of an anti-HIV agent. The two pharmaceutically acceptable dose units can optionally take the form of a single pharmaceutically acceptable dose unit. In some embodiments, the anti-HIV agent is one selected from the group consisting of a non-nucleoside reverse transcriptase inhibitor, a protease inhibitor, an entry or fusion inhibitor, and an integrase inhibitor.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 is a graph showing HIV-1 neutralization coverage curves for a number of anti-HIV bNAbs, including the new variant HCl/SAP10.

FIG. 2 is a graph showing polyreactivity of a number of anti-HIV bNAbs, as measured using the baculovirus-based assay described in Hötzel, I., et al., 2012, mAbs 4, 753-760.

FIG. 3 is a set of graphs showing that HCl/SAP10 had similar biodistribution and half-life in mice as 3BNC117 (a bNAb with a good half-life and tissue distribution).

### DETAILED DESCRIPTION OF THE INVENTION

This disclosure is based, at least in part, on unexpected discoveries that the disclosed variants of anti-HIV bNAb NIH45-46 exhibited improved biophysical properties, while retaining broad and potent neutralization activity. The anti-HIV bNAb variants and antigen-binding fragments as disclosed constitute a novel therapeutic strategy for treating and/or preventing HIV infection.

### A. Novel Broadly Neutralizing Anti-HIV Antibodies

a. Antibodies

The invention disclosed herein involves novel isolated broadly neutralizing anti-HIV antibodies, or antigen-binding portions thereof, with improved biophysical properties, such as reduced polyreactivity, prolonged half-life.

In some embodiments, the isolated anti-HIV antibody, or antigen-binding portion thereof, comprises: a heavy chain having a heavy chain amino acid sequence that is at least 75% (e.g., 80%, 85%, 90%, 95%, 96%, 97%, 98%, 99% or more) identical to SEQ ID NO: 1, wherein the heavy chain amino acid sequence comprises one or more heavy chain substitutions at one or more residues selected from the group consisting of S5, S21, G54, G55, V57, T68, T70, V73, S75, F79, S82, D85, V89, Y97, and P112; or a light chain having a light chain amino acid sequence that is at least 75% (e.g., 80%, 85%, 90%, 95%, 96%, 97%, 98%, 99% or more) identical to SEQ ID NO: 2, wherein the light chain amino acid sequence comprises one or more light chain substitutions at one or more residues selected from the group consisting of T5, S7, T10, S12, S14, T18, 120, S22, R24, Q27, S28, S30, R59, S61, S63, W65, D68, N70, S72, S74, and S78, wherein residue numbering is set forth with the Kabat numbering.

In some embodiments, the isolated anti-HIV antibody, or antigen-binding portion thereof, comprises: the heavy chain having the heavy chain polypeptide sequence that is at least 75% (e.g., 80%, 85%, 90%, 95%, 96%, 97%, 98%, 99% or more) identical to SEQ ID NO: 1, wherein the heavy chain amino acid sequence comprises the one or more heavy chain substitutions at the one or more residues selected from the group consisting of S5, S21, G54, G55, V57, T68, T70, V73, S75, F79, S82, D85, V89, Y97, and P112; and the light chain having the light chain amino acid sequence that is at least 75% (e.g., 80%, 85%, 90%, 95%, 96%, 97%, 98%, 99% or more) identical to SEQ ID NO: 2, wherein the light chain amino acid sequence comprises one or more light chain substitutions at one or more residues selected from the group consisting of T5, S7, T10, S12, S14, T18, 120, S22, R24, Q27, S28, S30, R59, S61, S63, W65, D68, N70, S72, S74, and S78.

In some embodiments, the one or more heavy chain substitutions comprise: a substitution of S5D or SSE, or a conservative substitution of Asp or Glu at residue S5; a substitution of S21D or S21E, or a conservative substitution of Asp or Glu at residue S21; a substitution of G54W or a conservative substitution of Trp at residue G54; a substitution of G55D or G55E, or a conservative substitution of Asp or Glu at residue G55; a substitution of V57D or V57E, or a conservative substitution of Asp or Glu at residue V57; a substitution of T68D or T68E, or a conservative substitution of Asp or Glu at residue T68; a substitution of T70D or T70E, or a conservative substitution of Asp or Glu at residue T70; a substitution of V73D or V73E, or a conservative substitution of Asp or Glu at residue V73; a substitution of S75D or S75E, or a conservative substitution of Asp or Glu at residue S75; a substitution of F79D, F79E, F79Y or F79H, or a conservative substitution of Asp, Glu, Tyr or His at residue F79; a substitution of S82D or S82E, or a conservative substitution of Asp or Glu at residue S82; a substitution of D85E, or a conservative substitution of Asp at residue D85; a substitution of V89D or V89E, or a conservative substitution of Asp or Glu at residue V89; a substitution of Y97D or Y97E, or a conservative substitution of Asp or Glu at residue Y97; a substitution of P112D or P112E, or a conservative substitution of Asp or Glu at residue P112; or a combination thereof.

In some embodiments, the one or more light chain substitutions comprises: a substitution of T5D or T5E, or a conservative substitution of Asp or Glu at residue T5; a substitution of S7D or STE, or a conservative substitution of Asp or Glu at residue S7; a substitution of T10D or T10E, or a conservative substitution of Asp or Glu at residue T10; a substitution of S12D or S12E, or a conservative substitution of Asp or Glu at residue S12; a substitution of S14D or S14E, or a conservative substitution of Asp or Glu at residue S14; a substitution of T18D or T18E, or a conservative substitution of Asp or Glu at residue T18; a substitution of 120D or 120E, or a conservative substitution of Asp or Glu at residue 120; a substitution of S22D or S22E, or a conservative substitution of Asp or Glu at residue S22; a substitution of R24D or R24E, or a conservative substitution of Asp or Glu at residue R24; a substitution of Q27D or Q27E, or a conservative substitution of Asp or Glu at residue Q27; a substitution of S28Y or S28H, or a conservative substitution of Tyr or His at residue S28; a substitution of S30D or S30E, or a conservative substitution of Asp or Glu at residue S30; a substitution of R59D or R59E, or a conservative substitution of Asp or Glu at residue R59; a substitution of S61D or S61E, or a conservative substitution of Asp or Glu at residue S61; a substitution of S63D or S63E, or a conservative substitution of Asp or Glu at residue S63; a substitution of W65D, W65E, W65Y or W65H, or a conservative substitution of Asp, Glu, Tyr or His at residue W65; a substitution of D68E, or a conservative substitution of Asp at residue D68; a substitution of N70D or N70E, or a conservative substitution of Asp or Glu at residue N70; a substitution of S72D or S72E, or a conservative substitution of Asp or Glu at residue S72; a substitution of S74D or S74E, or a conservative substitution of Asp or Glu at residue S74; a substitution of S78D or S78E, or a conservative substitution of Asp or Glu at residue S78; or a combination thereof.

In some embodiments, the one or more heavy chain substitutions further comprise substitutions of M428L and N434S.

In some embodiments, the light chain amino acid sequence comprises the one or more light chain substitutions of: (i) Q27E, S28H, and S30D; (ii) Q27E, S28H, S30D, and S74T; (iii) Q27E, S28H, S30D, S74T, M428L, and N434S; (iv) Q27E, S28Y, and S30D; (v) Q27D and S28H; (vi) S28Y; (vii) T5D, T10D, S12D, S14D, 120D, and S22D; or (viii) S61D, S63D, W65D, N70D, S72D, and S74D.

In some embodiments, the heavy chain amino acid sequence comprises the one or more heavy chain substitutions comprising G54W and T68D, and the light chain amino acid sequence comprises the one or more light chain substitutions of: (a) Q27E, S28H, S30D, and S74T; (b) Q27E, S28H, S30D, S74T, M428L, and N434S; (c) Q27E, S28Y, and S30D; (d) Q27D and S28H; or (e) S28Y.

In some embodiments, the heavy chain amino acid sequence comprises the one or more heavy chain substitutions comprising G54W; and the light chain amino acid sequence comprises the one or more light chain substitutions of: (a) Q27E, S28H, and S30D; (b) Q27E, S28Y, and S30D; or (c) Q27D and S28H.

In some embodiments, the heavy chain amino acid sequence comprises the amino acid sequence of SEQ ID NOs: 1 or 3. In some embodiments, the light chain amino acid sequence comprises an amino acid sequence of SEQ ID NOs: 2 and 4-10.

In some embodiments, the heavy chain amino acid sequence comprises the amino acid sequence of SEQ ID NO: 3-35. In some embodiments, the light chain amino acid sequence comprises an amino acid sequence of SEQ ID NOs: 2 and 36-80.

In some embodiments, the isolated anti-HIV antibody comprises a CDRH 1, a CDRH 2, a CDRH 3, a CDRL 1, a CDRL 2, and a CDRL 3, comprising respective sequences set forth in a CDR sequence set of: SEQ ID NOs: 81, 84, 87, 90, 93, and 96; SEQ ID NOs: 81, 84, 87, 99, 93, and 96; SEQ ID NOs: 81, 84, 87, 100, 93, and 96; SEQ ID NOs: 81, 84, 87, 101, 93, and 96; or SEQ ID NOs: 81, 84, 87, 102, 93, and 96; (set forth with the Kabat numbering); SEQ ID NOs: 82, 85, 88, 91, 94, and 97 (set forth with the IMGT numbering); or SEQ ID NOs: 83, 86, 89, 92, 95, and 98 (set forth with the Chothia numbering).

In some embodiments, the heavy chain amino acid sequence and the light chain amino acid sequence comprise the respective amino acid sequences set forth in a sequence set selected from the group consisting of SEQ ID NOs: 3 and 36; 3 and 38; 3 and 39; 4 and 2; 4 and 38; 4 and 39; 4 and 41; and 34 and 37.

Representative Sequences of Anti-HIV bNAbs

| SEQ | | |
|---|---|---|
| ID | | |
| NO | SEQUENCE | NOTES |
| 1 | QVRLSQSGGQMKKPGESMRLSCRASGYEFLNCPIN | NIH45-46 |
| | WIRLAPGRRPEWMGWLKPRGGAVNYARKFQGRVTM | HC |
| | TRDVYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMFIEALHNHYTQKSLSLSPG | |
| | K | |
| 2 | EIVLTQSPATLSLSPGETAIISCRTSQSGSLAWYQ | NIH45-46 |
| | QRPGQAPRLVIYSGSTRAAGIPDRFSGSRWGADYN | LC |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |

Representative Heavy Chain Amino
Acid Sequences of Variants

| | | |
|---|---|---|
| 3 | QVRLSQSGGQMKKPGESMRLSCRASGYEFLNCPIN | NIH45-46 |
| | WIRLAPGRRPEWMGWLKPRWGAVNYARKFQGRVTM | G54W HC |
| | TRDVYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 4 | QVRLSQSGGQMKKPGESMRLSCRASGYEFLNCPIN | HC1 HC |
| | WIRLAPGRRPEWMGWLKPRGGAVNYARKFQGRVDM | NIH45-46 |
| | TRDVYSDTAFLELRSLTSDDTAVYFCTRGKYCTAR | G54W |
| | DYYNWDFEHWGRGAPVTVSSASTKGPSVFPLAPSS | T68D |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALFTNHYTQKSLSLSPG | |
| | K | |
| 5 | QVRLSQSGGQMKKPGESMRLSCRASGYEFLNCPIN | NIH45-46 |
| | WIRLAPGRRPEWMGWLKPRGGAVNYARKFQGRVEM | G54W |
| | TRDVYSDTAFLELRSLTSDDTAVYFCTRGKYCTAR | T68E |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNFIYTQKSLSLSPG | |
| | K | |
| 6 | QVRLSQSGGQMKKPGESMRLSCRASGYEFLNCPIN | NIH45-46 |
| | WIRLAPGRRPEWMGWLKPRGGAVNYARKFQGRVTM | G54W |
| | DRDVYSDTAFLELRSLTSDDTAVYFCTRGKYCTAR | T70D |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVFTNAKTKPREEQYNSTYRVVSVL | |
| | TVLHQDWLNGKEYKCKVSNKALPAPIEKTISKAKG | |
| | QPREPQVYTLPPSREEMTKNQVSLTCLVKGFYPSD | |
| | IAVEWESNGQPENNYKTTPPVLDSDGSFFLYSKLT | |
| | VDKSRWQQGNVFSCSVMFTEALFTNFIYTQKSLSL | |
| | SPGK | |
| 7 | QVRLSQSGGQMKKPGESMRLSCRASGYEFLNCPIN | NIH45-46 |
| | WIRLAPGRRPEWMGWLKPRGGAVNYARKFQGRVTM | G54W |
| | ERDVYSDTAFLELRSLTSDDTAVYFCTRGKYCTAR | T70E |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMFfEALFfNHYTQKSLSLSP | |
| | GK | |
| 8 | QVRLSQSGGQMKKPGESMRLSCRASGYEFLNCPIN | NIH45-46 |
| | WIRLAPGRRPEWMGWLKPRWGAVNYARKFQGRVTM | G54W |
| | TRDVYDDTAFLELRSLTSDDTAVYFCTRGKYCTAR | S75D |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 9 | QVRLSQSGGQMKKPGESMRLSCRASGYEFLNCPIN | NIH45-46 |
| | WIRLAPGRRPEWMGWLKPRWGAVNYARKFQGRVTM | G54W |
| | TRDVYEDTAFLELRSLTSDDTAVYFCTRGKYCTAR | S75E |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 10 | QVRLSQSGGQMKKPGESMRLSCRASGYEFLNCPIN | NIH45-46 |
| | WIRLAPGRRPEWMGWLKPRWGAVNYARKFQGRVTM | G54W |
| | TRDVYSDTAFLELRDLTSDDTAVYFCTRGKYCTAR | S82D |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVET | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 11 | QVRLSQSGGQMKKPGESMRLSCRASGYEFLNCPIN | NIH45-46 |
| | WIRLAPGRRPEWMGWLKPRWGAVNYARKFQGRVTM | G54W |
| | TRDVYSDTAFLELRELTSDDTAVYFCTRGKYCTAR | S82E |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALFINHYTQKSLSLSPG | |
| | K | |
| 12 | QVRLSQSGGQMKKPGESMRLSCRASGYEFLNCPIN | NIH45-46 |
| | WIRLAPGRRPEWMGWLKPRWGAVNYARKFQGRVTM | G54W |
| | TRDVYSDTAFLELRSLTSEDTAVYFCTRGKYCTAR | D85E |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 13 | QVRLSQSGGQMKKPGESMRLSCRASGYEFLNCPIN | NIH45-46 |
| | WIRLAPGRRPEWMGWLKPRWGAVNYARKFQGRVTM | G54W |
| | TRDVYSDTAFLELRSLTSDDTADYFCTRGKYCTAR | V89D |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 14 | QVRLSQSGGQMKKPGESMRLSCRASGYEFLNCPIN | NIH45-46 |
| | WIRLAPGRRPEWMGWLKPRWGAVNYARKFQGRVTM | G54W |
| | TRDVYSDTAFLELRSLTSDDTAEYFCTRGKYCTAR | V89E |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMFTEALFTNFIYTQKSLSLS | |
| | PGK | |
| 15 | QVRLSQSGGQMKKPGESMRLSCRASGYEFLNCPIN | NIH45-46 |
| | WIRLAPGRRPEWMGWLKPRWGAVNYARKFQGRVTM | G54W |
| | TRDVYSDTAFLELRSLTSDDTAVYFCTRGKYCTAR | PU2D |
| | DYYNWDFEHWGRGADVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALFINHYTQKSLSLSPG | |
| | K | |
| 16 | QVRLSQSGGQMKKPGESMRLSCRASGYEFLNCPIN | NIH45-46 |
| | WIRLAPGRRPEWMGWLKPRWGAVNYARKFQGRVTM | G54W |
| | TRDVYSDTAFLELRSLTSDDTAVYFCTRGKYCTAR | P112E |
| | DYYNWDFEHWGRGAEVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 17 | QVRLSQSGGQMKKPGESMRLDCRASGYEFLNCPIN | NIH45-46 |
| | WIRLAPGRRPEWMGWLKPRGGAVNYARKFQGRVTM | S21D |
| | TRDVYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 18 | QVRLSQSGGQMKKPGESMRLECRASGYEFLNCPIN | NIH45-46 |
| | WIRLAPGRRPEWMGWLKPRGGAVNYARKFQGRVTM | S21E |
| | TRDVYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMFTEALFTNFIYTQKSLSLS | |
| | PGK | |
| 19 | QVRLSQSGGQMKKPGESMRLSCRASGYEFLNCPIN | NIH45-46 |
| | WIRLAPGRRPEWMGWLKPRGDAVNYARKFQGRVTM | G55D |
| | TRDVYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALFfNHYTQKSLSLSPG | |
| | K | |
| 20 | QVRLSQSGGQMKKPGESMRLSCRASGYEFLNCPIN | NIH45-46 |
| | WIRLAPGRRPEWMGWLKPRGDAVNYARKFQGRVTM | G55E |
| | TRDVYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 21 | QVRLSQSGGQMKKPGESMRLSCRASGYEFLNCPIN | NIH45-46 |
| | WIRLAPGRRPEWMGWLKPRGGADNYARKFQGRVTM | V57D |
| | TRDVYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 22 | QVRLSQSGGQMKKPGESMRLSCRASGYEFLNCPIN | NIH45-46 |
| | WIRLAPGRRPEWMGWLKPRGGAENYARKFQGRVTM | V57E |
| | TRDVYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVFTNAKTKPREEQYNSTYRVVSVL | |
| | TVLHQDWLNGKEYKCKVSNKALPAPIEKTISKAKG | |
| | QPREPQVYTLPPSREEMTKNQVSLTCLVKGFYPSD | |
| | IAVEWESNGQPENNYKTTPPVLDSDGSFFLYSKLT | |
| | VDKSRWQQGNVFSCSVMFTEALFTNFIYTQKSLSL | |
| | SPGK | |
| 23 | QVRLSQSGGQMKKPGESMRLSCRASGYEFLNCPIN | NIH45-46 |
| | WIRLAPGRRPEWMGWLKPRGGAVNYARKFQGRVTM | V73D |
| | TRDDYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 24 | QVRLSQSGGQMKKPGESMRLSCRASGYEFLNCPIN | NIH45-46 |
| | WIRLAPGRRPEWMGWLKPRGGAVNYARKFQGRVTM | V73E |
| | TRDEYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 25 | QVRLSQSGGQMKKPGESMRLSCRASGYEFLNCPIN | NIH45-46 |
| | WIRLAPGRRPEWMGWLKPRGGAVNYARKFQGRVTM | F79D |
| | TRDVYSDTADLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 26 | QVRLSQSGGQMKKPGESMRLSCRASGYEFLNCPIN | NIH45-46 |
| | WIRLAPGRRPEWMGWLKPRGGAVNYARKFQGRVTM | F79E |
| | TRDVYSDTAELELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVFTNAKTKPREEQYNSTYRVVSVL | |
| | TVLHQDWLNGKEYKCKVSNKALPAPIEKTISKAKG | |
| | QPREPQVYTLPPSREEMTKNQVSLTCLVKGFYPSD | |
| | IAVEWESNGQPENNYKTTPPVLDSDGSFFLYSKLT | |
| | VDKSRWQQGNVFSCSVMFTEALFTNFIYTQKSLSL | |
| | SPGK | |
| 27 | QVRLSQSGGQMKKPGESMRLSCRASGYEFLNCPIN | NIH45-46 |
| | WIRLAPGRRPEWMGWLKPRGGAVNYARKFQGRVTM | F79Y |
| | TRDVYSDTAYLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 28 | QVRLSQSGGQMKKPGESMRLSCRASGYEFLNCPIN | NIH45-46 |
| | WIRLAPGRRPEWMGWLKPRGGAVNYARKFQGRVTM | F79H |
| | TRDVYSDTAYLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 29 | QVRLSQSGGQMKKPGESMRLSCRASGYEFLNCPIN | NIH45-46 |
| | WIRLAPGRRPEWMGWLKPRGGAVNYARKFQGRVTM | Y97D |
| | TRDVYSDTAFLELRSLTSDDTAVYFCTRGKDCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 30 | QVRLSQSGGQMKKPGESMRLSCRASGYEFLNCPIN | NIH45-46 |
| | WIRLAPGRRPEWMGWLKPRGGAVNYARKFQGRVTM | Y97E |
| | TRDVYSDTAFLELRSLTSDDTAVYFCTRGKECTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVMHEALHNHYTQKSLSLSPGK | |
| 31 | QVRLDQSGGQMKKPGESMRLSCRASGYEFLNCPIN | NIH45-46 |
| | WIRLAPGRRPEWMGWLKPRGGAVNYARKFQGRVTM | S5D |
| | TRDVYSDTAFLELRSLTSDDTAVYFCTRGKYCTAR | |
| | DYYNWDFEHWGRGAPVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNV | |
| | NFLKPSNTKVDKRVEPKSCDKTHTCPPCPAPELLG | |
| | GPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPAPIEKTISKAKG | |
| | QPREPQVYTLPPSREEMTKNQVSLTCLVKGFYPSD | |
| | IAVEWESNGQPENNYKTTPPVLDSDGSFFLYSKLT | |
| | VDKSRWQQGNVFSCSVMFIEALPINHYTQKSLSLS | |
| | PGK | |
| 32 | QVRLEQSGGQMKKPGESMRLSCRASGYEFLNCPIN | NIH45-46 |
| | WIRLAPGRRPEWMGWLKPRGGAVNYARKFQGRVTM | S5E |
| | TRDVYSDTAFLELRSLTSDDTAVYFCTRGKYCTAR | |
| | DYYNWDFEFTWGRGAPVTVSSASTKGPSVFPLAPS | |
| | SKSTSGGTAALGCLVKDYFPEPVTVSWNSGALTSG | |
| | VHTFPAVLQSSGLYSLSSVVTVPSSSLGTQTYICN | |
| | VNHKPSNTKVDKRVEPKSCDKTHTCPPCPAPELLG | |
| | GPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPAPIEKTISKAKG | |
| | QPREPQVYTLPPSREEMTKNQVSLTCLVKGFYPSD | |
| | IAVEWESNGQPENNYKTTPPVLDSDGSFFLYSKLT | |
| | VDKSRWQQGNVFSCSVMHEALHNHYTQKSLSLSPG | |
| | K | |
| 33 | QVRLSQSGGQMKKPGESMRLSCRASGYEFLNCPIN | NIH45-46 |
| | WIRLAPGRRPEWMGWLKPRGGAVNYARKFQGRVTM | with M428L |
| | TRDVYSDTAFLELRSLTSDDTAVYFCTRGKYCTAR | and N343S |
| | DYYNWDFEFTWGRGAPVTVSSASTKGPSVFPLAPS | |
| | SKSTSGGTAALGCLVKDYFPEPVTVSWNSGALTSG | |
| | VHTFPAVLQSSGLYSLSSVVTVPSSSLGTQTYICN | |
| | VNHKPSNTKVDKRVEPKSCDKTHTCPPCPAPELLG | |
| | GPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPAPIEKTISKAKG | |
| | QPREPQVYTLPPSREEMTKNQVSLTCLVKGFYPSD | |
| | IAVEWESNGQPENNYKTTPPVLDSDGSFFLYSKLT | |
| | VDKSRWQQGNVFSCSVLHEALHSHYTQKSLSLSPG | |
| | K | |
| 34 | QVRLSQSGGQMKKPGESMRLSCRASGYEFLNCPIN | HC1 HC- |
| | WIRLAPGRRPEWMGWLKPRGGAVNYARKFQGRVDM | LS |
| | TRDVYSDTAFLELRSLTSDDTAVYFCTRGKYCTAR | NIH45-46 |
| | DYYNWDFEHWGRGAPVTVSSASTKGPSVFPLAPSS | G54W, |
| | KSTSGGTAALGCLVKDYFPEPVTVSWNSGALTSGV | T68D, |
| | HTFPAVLQSSGLYSLSSVVTVPSSSLGTQTYICNV | M428L and |
| | NHKPSNTKVDKRVEPKSCDKTHTCPPCPAPELLGG | N343S |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVLHEALHSHYTQKSLSLSPGK | |
| 35 | QVRLSQSGGQMKKPGESMRLSCRASGYEFLNCPIN | NIH45-46- |
| | WIRLAPGRRPEWMGWLKPRGGAVNYARKFQGRVEM | LS |
| | TRDVYSDTAFLELRSLTSDDTAVYFCTRGKYCTAR | G54W, |
| | DYYNWDFEHWGRGAPVTVSSASTKGPSVFPLAPSS | T68E, |
| | KSTSGGTAALGCLVKDYFPEPVTVSWNSGALTSGV | M428L and |
| | HTFPAVLQSSGLYSLSSVVTVPSSSLGTQTYICNV | N343S |
| | NHKPSNTKVDKRVEPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSVLT | |
| | VLHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLTV | |
| | DKSRWQQGNVFSCSVLHEALHSHYTQKSLSLSPGK | |

Representative Light Chain
Amino Acid Sequences

| | | |
|---|---|---|
| 36 | EIVLTQSPATLSLSPGETAIISCRTSEHGDLAWYQ | SAP 10 LC |
| | QRPGQAPRLVIYSGSTRAAGIPDRFSGSRWGADYN | (NIH45-46 |
| | LSISNLESGDFGVYYCQQYEFFGQGTKVQVDIKRT | LC with |
| | VAAPSVFIFPPSDEQLKSGTASVVCLLNNFYPREA | Q27E, |
| | KVQWKVDNALQSGNSQESVTEQDSKDSTYSLSSTL | S28H, and |
| | TLSKADYEKHKVYACEVTHQGLSSPVTKSFNRGEC | S30D) |
| 37 | EIVLTQSPATLSLSPGETAIISCRTSEHGDLAWYQ | SAP10T |
| | QRPGQAPRLVIYSGSTRAAGIPDRFSGSRWGADYN | LC(NIH45- |
| | LSITNLESGDFGVYYCQQYEFFGQGTKVQVDIKRT | 46 LC with |
| | VAAPSVFIFPPSDEQLKSGTASVVCLLNNFYPREA | Q27E, |
| | KVQWKVDNALQSGNSQESVTEQDSKDSTYSLSSTL | S28H, |
| | TLSKADYEKHKVYACEVTFIQGLSSPVTKSFNRGE | S30D, and |
| | C | S74T) |
| 38 | EIVLTQSPATLSLSPGETAIISCRTSEYGDLAWYQ | SAP3 LC |
| | QRPGQAPRLVIYSGSTRAAGIPDRFSGSRWGADYN | (NIH45-46 |
| | LSISNLESGDFGVYYCQQYEFFGQGTKVQVDIKRT | LC with |
| | VAAPSVFIFPPSDEQLKSGTASVVCLLNNFYPREA | Q27E, |
| | KVQWKVDNALQSGNSQESVTEQDSKDSTYSLSSTL | S28Y, and |
| | TLSKADYEKHKVYACEVTHQGLSSPVTKSFNRGEC | S30D) |
| 39 | EIVLTQSPATLSLSPGETAHSCRTSDHGSLAWYQQ | SAP8 LC |
| | RPGQAPRLVIYSGSTRAAGIPDRFSGSRWGADYNL | (NIH45-46 |
| | SISNLESGDFGVYYCQQYEFFGQGTKVQVDIKRTV | LC with |
| | AAPSVFIFPPSDEQLKSGTASVVCLLNNFYPREAK | Q27D, |
| | VQWKVDNALQSGNSQESVTEQDSKDSTYSLSSTLT | S28H) |
| | LSKADYEKHKVYACEVTHQGLSSPVTKSFNRGEC | |
| 40 | EIVLTQSPATLSLSPGETAIISCRTSEHGSLAWYQ | XYZ1 LC |
| | QRPGQAPRLVIYSGSTRAAGIPDRFSGSRWGADYN | (NIH45-46 |
| | LSTSNLESGDFGVYYCQQYEFFGQGTKVQVDIKRT | LC with |
| | VAAPSVFIFPPSDEQLKSGTASVVCLLNNFYPREA | Q27E, |
| | KVQWKVDNALQSGNSQESVTEQDSKDSTYSLSSTL | S28H) |
| | TLSKADYEKHKVYACEVTHQGLSSPVTKSFNRGEC | |
| 41 | EIVLTQSPATLSLSPGETAIISCRTSQYGSLAWYQ | m2 LC |
| | QRPGQAPRLVIYSGSTRAAGIPDRFSGSRWGADYN | (NIH45-46 |
| | LSISNLESGDFGVYYCQQYEFFGQGTKVQVDIKRT | LC with |
| | VAAPSVFIFPPSDEQLKSGTASVVCLLNNFYPREA | S28Y) |
| | KVQWKVDNALQSGNSQESVTEQDSKDSTYSLSSTL | |
| | TLSKADYEKHKVYACEVTHQGLSSPVTKSFNRGEC | |
| 42 | EIVLDQSPATLSLSPGETAIISCRTSQSGSLAWYQ | NIH45-46 |
| | QRPGQAPRLVIYSGSTRAAGIPDRFSGSRWGADYN | T5D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFIKVYACEVTHQGLSSPVTKSFNRGE | |
| | C | |
| 43 | EIVLEQSPATLSLSPGETAIISCRTSQSGSLAWYQ | NIH45-46 |
| | QRPGQAPRLVIYSGSTRAAGIPDRFSGSRWGADYN | T5E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 44 | EIVLTQDPATLSLSPGETAIISCRTSQSGSLAWYQ | NIH45-46 |
| | QRPGQAPRLVIYSGSTRAAGIPDRFSGSRWGADYN | S7D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 45 | EIVLTQEPATLSLSPGETAIISCRTSQSGSLAWYQ | NIH45-46 |
| | QRPGQAPRLVIYSGSTRAAGIPDRFSGSRWGADYN | S7E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 46 | EIVLTQSPADLSLSPGETAIISCRTSQSGSLAWYQ | NIH45-46 |
| | QRPGQAPRLVIYSGSTRAAGIPDRFSGSRWGADYN | T10D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFTKVYACEVTHQGLSSPVTKSFNRGE | |
| | C | |
| 47 | EIVLTQSPAELSLSPGETAIISCRTSQSGSLAWYQ | NIH45-46 |
| | QRPGQAPRLVIYSGSTRAAGIPDRFSGSRWGADYN | T10E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 48 | EIVLTQSPATLDLSPGETAIISCRTSQSGSLAWYQ | NIH45-46 |
| | QRPGQAPRLVIYSGSTRAAGIPDRFSGSRWGADYN | S12D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 49 | EIVLTQSPATLELSPGETAIISCRTSQSGSLAWYQ | NIH45-46 |
| | QRPGQAPRLVIYSGSTRAAGIPDRFSGSRWGADYN | S12E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFTKVYACEVTHQGLSSPVTKSFNRGE | |
| | C | |
| 50 | EIVLTQSPATLSLDPGETAIISCRTSQSGSLAWYQ | NIH45-46 |
| | QRPGQAPRLVIYSGSTRAAGIPDRFSGSRWGADYN | S14D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFIKVYACEVTHQGLSSPVTKSFNRGE | |
| | C | |
| 51 | EIVLTQSPATLSLEPGETAIISCRTSQSGSLAWYQ | NIH45-46 |
| | QRPGQAPRLVIYSGSTRAAGIPDRFSGSRWGADYN | S14E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 52 | EIVLTQSPATLSLSPGEDAIISCRTSQSGSLAWYQ | NIH45-46 |
| | QRPGQAPRLVIYSGSTRAAGIPDRFSGSRWGADYN | TI8D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 53 | EIVLTQSPATLSLSPGEEAIISCRTSQSGSLAWYQ | NIH45-46 |
| | QRPGQAPRLVIYSGSTRAAGIPDRFSGSRWGADYN | TI8E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFIKVYACEVTHQGLSSPVTKSFNRGE | |
| | C | |
| 54 | EIVLTQSPATLSLSPGETADISCRTSQSGSLAWYQ | NIH45-46 |
| | QRPGQAPRLVIYSGSTRAAGIPDRFSGSRWGADYN | I20D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 55 | EIVLTQSPATLSLSPGETAEISCRTSQSGSLAWYQ | NIH45-46 |
| | QRPGQAPRLVIYSGSTRAAGIPDRFSGSRWGADYN | I20E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 56 | EIVLTQSPATLSLSPGETAIIDCRTSQSGSLAWYQ | NIH45-46 |
| | QRPGQAPRLVIYSGSTRAAGIPDRFSGSRWGADYN | S22D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 57 | EIVLTQSPATLSLSPGETAIIECRTSQSGSLAWYQ | NIH45-46 |
| | QRPGQAPRLVIYSGSTRAAGIPDRFSGSRWGADYN | S22E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 58 | EIVLTQSPATLSLSPGETAIISCDTSQSGSLAWYQ | NIH45-46 |
| | QRPGQAPRLVIYSGSTRAAGIPDRFSGSRWGADYN | R24D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFTKVYACEVTHQGLSSPVTKSFNRGE | |
| | C | |
| 59 | EIVLTQSPATLSLSPGETAIISCETSQSGSLAWYQ | NIH45-46 |
| | QRPGQAPRLVIYSGSTRAAGIPDRFSGSRWGADYN | R24E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFIKVYACEVTHQGLSSPVTKSFNRGE | |
| | C | |
| 60 | EIVLTQSPATLSLSPGETAIISCRTSQSGSLAWYQ | NIH45-46 |
| | QRPGQAPRLVIYSGSTRAAGIPDDFSGSRWGADYN | R59D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 61 | EIVLTQSPATLSLSPGETAIISCRTSQSGSLAWYQ | NIH45-46 |
| | QRPGQAPRLVIYSGSTRAAGIPDEFSGSRWGADYN | R59E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 62 | EIVLTQSPATLSLSPGETAIISCRTSQSGSLAWYQ | NIH45-46 |
| | QRPGQAPRLVIYSGSTRAAGIPDRFDGSRWGADYN | S61D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFIKVYACEVTHQGLSSPVTKSFNRGE | |
| | C | |
| 63 | EIVLTQSPATLSLSPGETAIISCRTSQSGSLAWYQ | NIH45-46 |
| | QRPGQAPRLVIYSGSTRAAGIPDRFEGSRWGADYN | S61E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 64 | EIVLTQSPATLSLSPGETAIISCRTSQSGSLAWYQ | NIH45-46 |
| | QRPGQAPRLVIYSGSTRAAGIPDRFSGDRWGADYN | S63D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 65 | EIVLTQSPATLSLSPGETAIISCRTSQSGSLAWYQ | NIH45-46 |
| | QRPGQAPRLVIYSGSTRAAGIPDRFSGERWGADYN | S63E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 66 | EIVLTQSPATLSLSPGETAIISCRTSQSGSLAWYQ | NIH45-46 |
| | QRPGQAPRLVIYSGSTRAAGIPDRFSGSRYGADYN | W65Y |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 67 | EIVLTQSPATLSLSPGETAIISCRTSQSGSLAWYQ | NIH45-46 |
| | QRPGQAPRLVIYSGSTRAAGIPDRFSGSRHGADYN | W65H |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFTKVYACEVTHQGLSSPVTKSFNRGE | |
| | C | |
| 68 | EIVLTQSPATLSLSPGETAIISCRTSQSGSLAWYQ | NIH45-46 |
| | QRPGQAPRLVIYSGSTRAAGIPDRFSGSRDGADYN | W65D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 69 | EIVLTQSPATLSLSPGETAIISCRTSQSGSLAWYQ | NIH45-46 |
| | QRPGQAPRLVIYSGSTRAAGIPDRFSGSREGADYN | W65E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 70 | EIVLTQSPATLSLSPGETAIISCRTSQSGSLAWYQ | NIH45-46 |
| | QRPGQAPRLVIYSGSTRAAGIPDRFSGSRWGAEYN | D68E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 71 | EIVLTQSPATLSLSPGETAIISCRTSQSGSLAWYQ | NIH45-46 |
| | QRPGQAPRLVIYSGSTRAAGIPDRFSGSRWGADYD | N70D |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFIKVYACEVTHQGLSSPVTKSFNRGE | |
| | C | |
| 72 | EIVLTQSPATLSLSPGETAIISCRTSQSGSLAWYQ | NIH45-46 |
| | QRPGQAPRLVIYSGSTRAAGIPDRFSGSRWGADYE | N70E |
| | LSISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 73 | EIVLTQSPATLSLSPGETAIISCRTSQSGSLAWYQ | NIH45-46 |
| | QRPGQAPRLVIYSGSTRAAGIPDRFSGSRWGADYN | S72D |
| | LDISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 74 | EIVLTQSPATLSLSPGETAIISCRTSQSGSLAWYQ | NIH45-46 |
| | QRPGQAPRLVIYSGSTRAAGIPDRFSGSRWGADYN | S72E |
| | LEISNLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 75 | EIVLTQSPATLSLSPGETAIISCRTSQSGSLAWYQ | NIH45-46 |
| | QRPGQAPRLVIYSGSTRAAGIPDRFSGSRWGADYN | S74D |
| | LSIDNLESGDFGVYYCQQYEFFGQGTKVQVDIKRT | |
| | VAAPSVFIFPPSDEQLKSGTASVVCLLNNFYPREA | |
| | RVQWRVDNALQSGNSQESVTEQDSRDSTYSLSSTL | |
| | TLSKADYEKHKVYACEVTHQGLSSPVTKSFNRGEC | |
| 76 | EIVLTQSPATLSLSPGETAIISCRTSQSGSLAWYQ | NIH45-46 |
| | QRPGQAPRLVIYSGSTRAAGIPDRFSGSRWGADYN | S74E |
| | LSIENLESGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKFTKVYACEVTHQGLSSPVTKSFNRGE | |
| | C | |
| 77 | EIVLTQSPATLSLSPGETAIISCRTSQSGSLAWYQ | NIH45-46 |
| | QRPGQAPRLVIYSGSTRAAGIPDRFSGSRWGADYN | S78D |
| | LSISNLEDGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSRADYERHRVYACEVTHQGLSSPVTRSFNRGEC | |
| 78 | EIVLTQSPATLSLSPGETAIISCRTSQSGSLAWYQ | NIH45-46 |
| | QRPGQAPRLVIYSGSTRAAGIPDRFSGSRWGADYN | S78E |
| | LSISNLEEGDFGVYYCQQYEFFGQGTKVQVDIKRT | |
| | VAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |
| 79 | EIVLDQSPADLDLDPGETADIDCRTSQSGSLAWYQ | NIH45-46 |
| | QRPGQAPRLVIYSGSTRAAGIPDRFSGSRWGADYN | T5D, T10D, |
| | LSISNLESGDFGVYYCQQYEFFGQGTKVQVDIKRT | S12D, |
| | VAAPSVFIFPPSDEQLKSGTASVVCLLNNFYPREA | S14D, |
| | RVQWRVDNALQSGNSQESVTEQDSRDSTYSLSSTL | I20D, |
| | TLSKADYEKHKVYACEVTHQGLSSPVTKSFNRGEC | S22D |
| 80 | EIVLTQSPATLSLSPGETAIISCRTSQSGSLAWYQ | NIH45-46 |
| | QRPGQAPRLVIYSGSTRAAGIPDRFDGDRDGADYD | S61D, |
| | LDIDNLESGDFGVYYCQQYEFFGQGTKVQVDIKRT | S63D, |
| | VAAPSVFIFPPSDEQLKSGTASVVCLLNNFYPREA | W65D, |
| | RVQWRVDNALQSGNSQESVTEQDSRDSTYSLSSTL | N70D, |
| | TLSKADYEKHKVYACEVTHQGLSSPVTKSFNRGEC | S72D, S74D |

Representative CDR Amino Acid
Sequences of Variants

| | | |
|---|---|---|
| 81 | NCPIN | CDRH 1 |
| | | (Kabat) |
| 82 | GYEFLNCP | CDRH1 |
| | | (IMGT) |
| 83 | GYEFLN | CDRH1 |
| | | (Chothia) |
| 84 | WLKPRWGAVNYARKFQG | CDRH 2 |
| | | (Kabat) |
| 85 | LKPRWGAV | CDRH 2 |
| | | (IMGT) |
| 86 | KPRWGA | CDRH 2 |
| | | (Chothia) |
| 87 | GKYCTARDYYNWDFEH | CDRH 3 |
| | | (Kabat) |
| 88 | TRGKYCTARDYYNWDFEH | CDRH 3 |
| | | (IMGT) |
| 89 | GKYCTARDYYNWDFEH | CDRH 3 |
| | | (Chothia) |
| 90 | RTSQSGSLA | CDRL 1 |
| | | (Rabat) |
| 91 | QYGS | CDRL 1 |
| | | (IMGT) |
| 92 | RTSQYGSLA | CDRL 1 |
| | | (Chothia) |
| 93 | SGSTRAA | CDRL 2 |
| | | (Rabat) |
| 94 | SGS | CDRL 2 |
| | | (IMGT) |
| 95 | SGSTRAA | CDRL 2 |
| | | (Chothia) |
| 96 | QQYEF | CDRL 3 |
| | | (Rabat) |
| 97 | QQYEF | CDRL 3 |
| | | (IMGT) |
| 98 | QQYEF | CDRL 3 |
| | | (Chothia) |
| 99 | RTSQYGSLA | m2 CDRL 1 |
| 100 | RTSEHGDLA | SAP 10 |
| | | CDRL 1 |
| 101 | RTSEYGDLA | SAP3 |
| | | CDRL 1 |
| 102 | RTSDHGSLA | SAP8 |
| | | CDRL 1 |

Representative Nucleotide
Sequences of Variants

| | | |
|---|---|---|
| 103 | CAAGTGCGACTGTCGCAGTCTGGAGGTCAGATGAA | NIH45-46 |
| | GAAGCCTGGCGAGTCGATGAGACTTTCCTGTCGGG | HC |
| | CTTCCGGATATGAATTTCTGAATTGTCCAATAAAT | |
| | TGGATTCGCCTGGCCCCCGGAAGACGGCCTGAGTG | |
| | GATGGGATGGCTGAAGCCTAGGGGAGGGGCCGTCA | |
| | ATTACGCACGTAAATTTCAGGGCAGAGTGACCATG | |
| | ACTCGAGACGTGTATTCCGACACAGCCTTTTTGGA | |
| | GTTGCGCTCCTTGACATCAGACGACACGGCCGTCT | |
| | ATTTTTGTACTAGGGGAAAATATTGTACTGCGCGC | |
| | GACTATTATAATTGGGACTTCGAACACTGGGGCCG | |
| | GGGTGCCCCGGTCACCGTCTCATCAGCGTCGACCA | |
| | AGGGCCCATCGGTCTTCCCCCTGGCACCCTCCTCC | |
| | AAGAGCACCTCTGGGGGCACAGCGGCCCTGGGCTG | |
| | CCTGGTCAAGGACTACTTCCCCGAACCTGTGACGG | |
| | TCTCGTGGAACTCAGGCGCCCTGACCAGCGGCGTG | |
| | CACACCTTCCCGGCTGTCCTACAGTCCTCAGGACT | |
| | CTACTCCCTCAGCAGCGTGGTGACCGTGCCCTCCA | |
| | GCAGCTTGGGCACCCAGACCTACATCTGCAACGTG | |
| | AATCACAAGCCCAGCAACACCAAGGTGGACAAGAG | |
| | AGTTGAGCCCAAATCTTGTGACAAAACTCACACAT | |
| | GCCCACCGTGCCCAGCACCTGAACTCCTGGGGGGA | |
| | CCGTCAGTCTTCCTCTTCCCCCCAAAACCCAAGGA | |
| | CACCCTCATGATCTCCCGGACCCCTGAGGTCACAT | |
| | GCGTGGTGGTGGACGTGAGCCACGAAGACCCTGAG | |
| | GTCAAGTTCAACTGGTACGTGGACGGCGTGGAGGT | |
| | GCATAATGCCAAGACAAAGCCGCGGGAGGAGCAGT | |
| | ACAACAGCACGTACCGTGTGGTCAGCGTCCTCACC | |
| | GTCCTGCACCAGGACTGGCTGAATGGCAAGGAGTA | |
| | CAAGTGCAAGGTCTCCAACAAAGCCCTCCCAGCCC | |
| | CCATCGAGAAAACCATCTCCAAAGCCAAAGGGCAG | |
| | CCCCGAGAACCACAGGTGTACACCCTGCCCCCATC | |
| | CCGGGAGGAGATGACCAAGAACCAGGTCAGCCTGA | |
| | CCTGCCTGGTCAAAGGCTTCTATCCCAGCGACATC | |
| | GCCGTGGAGTGGGAGAGCAATGGGCAGCCGGAGAA | |
| | CAACTACAAGACCACGCCTCCCGTGCTGGACTCCG | |
| | ACGGCTCCTTCTTCCTCTATAGCAAGCTCACCGTG | |
| | GACAAGAGCAGGTGGCAGCAGGGGAACGTCTTCTC | |
| | ATGCTCCGTGATGCATGAGGCTCTGCACAACCACT | |
| | ACACGCAGAAGAGCCTCTCCCTGTCCCCGGGTAAA | |
| 104 | CAAGTGCGACTGTCGCAGTCTGGAGGTCAGATGAA | NIH45-46 |
| | GAAGCCTGGCGAGTCGATGAGACTTTCCTGTCGGG | G54W HC |
| | CTTCCGGATATGAATTTCTGAATTGTCCAATAAAT | |
| | TGGATTCGCCTGGCCCCCGGAAGACGGCCTGAGTG | |
| | GATGGGATGGCTGAAGCCTAGGTGGGGGGCCGTCA | |
| | ATTACGCACGTAAATTTCAGGGCAGAGTGACCATG | |
| | ACTCGAGACGTGTATTCCGACACAGCCTTTTTGGA | |
| | GTTGCGCTCCTTGACATCAGACGACACGGCCGTCT | |
| | ATTTTTGTACTAGGGGAAAATATTGTACTGCGCGC | |
| | GACTATTATAATTGGGACTTCGAACACTGGGGCCG | |
| | GGGTGCCCCGGTCACCGTCTCATCAGCGTCGACCA | |
| | AGGGCCCATCGGTCTTCCCCCTGGCACCCTCCTCC | |
| | AAGAGCACCTCTGGGGGCACAGCGGCCCTGGGCTG | |
| | CCTGGTCAAGGACTACTTCCCCGAACCTGTGACGG | |
| | TCTCGTGGAACTCAGGCGCCCTGACCAGCGGCGTG | |
| | CACACCTTCCCGGCTGTCCTACAGTCCTCAGGACT | |
| | CTACTCCCTCAGCAGCGTGGTGACCGTGCCCTCCA | |
| | GCAGCTTGGGCACCCAGACCTACATCTGCAACGTG | |
| | AATCACAAGCCCAGCAACACCAAGGTGGACAAGAG | |
| | AGTTGAGCCCAAATCTTGTGACAAAACTCACACAT | |
| | GCCCACCGTGCCCAGCACCTGAACTCCTGGGGGGA | |
| | CCGTCAGTCTTCCTCTTCCCCCCAAAACCCAAGGA | |
| | CACCCTCATGATCTCCCGGACCCCTGAGGTCACAT | |
| | GCGTGGTGGTGGACGTGAGCCACGAAGACCCTGAG | |
| | GTCAAGTTCAACTGGTACGTGGACGGCGTGGAGGT | |
| | GCATAATGCCAAGACAAAGCCGCGGGAGGAGCAGT | |
| | ACAACAGCACGTACCGTGTGGTCAGCGTCCTCACC | |
| | GTCCTGCACCAGGACTGGCTGAATGGCAAGGAGTA | |
| | CAAGTGCAAGGTCTCCAACAAAGCCCTCCCAGCCC | |
| | CCATCGAGAAAACCATCTCCAAAGCCAAAGGGCAG | |
| | CCCCGAGAACCACAGGTGTACACCCTGCCCCCATC | |
| | CCGGGAGGAGATGACCAAGAACCAGGTCAGCCTGA | |
| | CCTGCCTGGTCAAAGGCTTCTATCCCAGCGACATC | |
| | GCCGTGGAGTGGGAGAGCAATGGGCAGCCGGAGAA | |
| | CAACTACAAGACCACGCCTCCCGTGCTGGACTCCG | |
| | ACGGCTCCTTCTTCCTCTATAGCAAGCTCACCGTG | |
| | GACAAGAGCAGGTGGCAGCAGGGGAACGTCTTCTC | |
| | ATGCTCCGTGATGCATGAGGCTCTGCACAACCACT | |
| | ACACGCAGAAGAGCCTCTCCCTGTCCCCGGGTAAA | |
| 105 | CAAGTGCGACTGTCGCAGTCTGGAGGTCAGATGAA | HC1 HC |
| | GAAGCCTGGCGAGTCGATGAGACTTTCCTGTCGGG | (NIH45-46 |
| | CTTCCGGATATGAATTTCTGAATTGTCCAATAAAT | G54W HC |
| | TGGATTCGCCTGGCCCCCGGAAGACGGCCTGAGTG | with T68D) |
| | GATGGGATGGCTGAAGCCTAGGTGGGGGGCCGTCA | |
| | ATTACGCACGTAAATTTCAGGGCAGAGTGGACATG | |
| | ACTCGAGACGTGTATTCCGACACAGCCTTTTTGGA | |
| | GTTGCGCTCCTTGACATCAGACGACACGGCCGTCT | |
| | ATTTTTGTACTAGGGGAAAATATTGTACTGCGCGC | |
| | GACTATTATAATTGGGACTTCGAACACTGGGGCCG | |
| | GGGTGCCCCGGTCACCGTCTCATCAGCGTCGACCA | |
| | AGGGCCCATCGGTCTTCCCCCTGGCACCCTCCTCC | |
| | AAGAGCACCTCTGGGGGCACAGCGGCCCTGGGCTG | |
| | CCTGGTCAAGGACTACTTCCCCGAACCTGTGACGG | |
| | TCTCGTGGAACTCAGGCGCCCTGACCAGCGGCGTG | |
| | CACACCTTCCCGGCTGTCCTACAGTCCTCAGGACT | |
| | CTACTCCCTCAGCAGCGTGGTGACCGTGCCCTCCA | |
| | GCAGCTTGGGCACCCAGACCTACATCTGCAACGTG | |
| | AATCACAAGCCCAGCAACACCAAGGTGGACAAGAG | |
| | AGTTGAGCCCAAATCTTGTGACAAAACTCACACAT | |
| | GCCCACCGTGCCCAGCACCTGAACTCCTGGGGGGA | |
| | CCGTCAGTCTTCCTCTTCCCCCCAAAACCCAAGGA | |
| | CACCCTCATGATCTCCCGGACCCCTGAGGTCACAT | |
| | GCGTGGTGGTGGACGTGAGCCACGAAGACCCTGAG | |
| | GTCAAGTTCAACTGGTACGTGGACGGCGTGGAGGT | |
| | GCATAATGCCAAGACAAAGCCGCGGGAGGAGCAGT | |
| | ACAACAGCACGTACCGTGTGGTCAGCGTCCTCACC | |
| | GTCCTGCACCAGGACTGGCTGAATGGCAAGGAGTA | |
| | CAAGTGCAAGGTCTCCAACAAAGCCCTCCCAGCCC | |
| | CCATCGAGAAAACCATCTCCAAAGCCAAAGGGCAG | |
| | CCCCGAGAACCACAGGTGTACACCCTGCCCCCATC | |
| | CCGGGAGGAGATGACCAAGAACCAGGTCAGCCTGA | |
| | CCTGCCTGGTCAAAGGCTTCTATCCCAGCGACATC | |
| | GCCGTGGAGTGGGAGAGCAATGGGCAGCCGGAGAA | |
| | CAACTACAAGACCACGCCTCCCGTGCTGGACTCCG | |
| | ACGGCTCCTTCTTCCTCTATAGCAAGCTCACCGTG | |
| | GACAAGAGCAGGTGGCAGCAGGGGAACGTCTTCTC | |
| | ATGCTCCGTGATGCATGAGGCTCTGCACAACCACT | |
| | ACACGCAGAAGAGCCTCTCCCTGTCCCCGGGTAAA | |
| 106 | GAAATTGTGTTGACACAGTCTCCAGCCACCCTGTC | SAP 10 LC |
| | TTTGTCTCCAGGGGAAACAGCCATCATCTCTTGTC | (NIH45-46 |
| | GGACCAGTGAGCATGGTGACTTAGCCTGGTATCAA | LC with |
| | CAGAGGCCCGGCCAGGCCCCCAGGCTCGTCATCTA | Q27E, |
| | TTCGGGTTCTACTCGGGCCGCTGGCATCCCAGACA | S28H, and |
| | GGTTCAGCGGCAGTCGGTGGGGGGCAGACTACAAT | S30D) |
| | CTCAGCATCAGCAACCTGGAGTCGGGAGATTTTGG | |
| | TGTTTATTATTGTCAGCAGTATGAATTTTTTGGCC | |
| | AGGGGACCAAGGTCCAGGTCGACATCAAACGTACG | |
| | GTGGCTGCACCATCTGTCTTCATCTTCCCGCCATC | |
| | TGATGAGCAGTTGAAATCTGGAACTGCCTCTGTTG | |
| | TGTGCCTGCTGAATAACTTCTATCCCAGAGAGGCC | |
| | AAAGTACAGTGGAAGGTGGATAACGCCCTCCAATC | |
| | GGGTAACTCCCAGGAGAGTGTCACAGAGCAGGACA | |
| | GCAAGGACAGCACCTACAGCCTCAGCAGCACCCTG | |
| | ACGCTGAGCAAAGCAGACTACGAGAAACACAAAGT | |
| | CTACGCCTGCGAAGTCACCCATCAGGGCCTGAGCT | |
| | CGCCCGTCACAAAGAGCTTCAACAGGGGAGAGTGT | |
| 107 | GAAATTGTGTTGACACAGTCTCCAGCCACCCTGTC | SAP10T |
| | TTTGTCTCCAGGGGAAACAGCCATCATCTCTTGTC | LC (NIH45- |
| | GGACCAGTGAGCATGGTGACTTAGCCTGGTATCAA | 46 LC with |
| | CAGAGGCCCGGCCAGGCCCCCAGGCTCGTCATCTA | Q27E, |
| | TTCGGGTTCTACTCGGGCCGCTGGCATCCCAGACA | S28H, |
| | GGTTCAGCGGCAGTCGGTGGGGGGCAGACTACAAT | S30D, and |
| | CTCAGCATCAcCAACCTGGAGTCGGGAGATTTTGG | S74T) |
| | TGTTTATTATTGTCAGCAGTATGAATTTTTTGGCC | |
| | AGGGGACCAAGGTCCAGGTCGACATCAAACGTACG | |
| | GTGGCTGCACCATCTGTCTTCATCTTCCCGCCATC | |
| | TGATGAGCAGTTGAAATCTGGAACTGCCTCTGTTG | |
| | TGTGCCTGCTGAATAACTTCTATCCCAGAGAGGCC | |
| | AAAGTACAGTGGAAGGTGGATAACGCCCTCCAATC | |
| | GGGTAACTCCCAGGAGAGTGTCACAGAGCAGGACA | |
| | GCAAGGACAGCACCTACAGCCTCAGCAGCACCCTG | |
| | ACGCTGAGCAAAGCAGACTACGAGAAACACAAAGT | |
| | CTACGCCTGCGAAGTCACCCATCAGGGCCTGAGCT | |
| | CGCCCGTCACAAAGAGCTTCAACAGGGGAGAGTGT | |
| 108 | GAAATTGTGTTGACACAGTCTCCAGCCACCCTGTC | SAP3 LC |
| | TTTGTCTCCAGGGGAAACAGCCATCATCTCTTGTC | (NIH45-46 |
| | GGACCAGTGAGTATGGTGACTTAGCCTGGTATCAA | LC with |
| | CAGAGGCCCGGCCAGGCCCCCAGGCTCGTCATCTA | Q27E, |
| | TTCGGGTTCTACTCGGGCCGCTGGCATCCCAGACA | S28Y, and |
| | GGTTCAGCGGCAGTCGGTGGGGGGCAGACTACAAT | S30D) |
| | CTCAGCATCAGCAACCTGGAGTCGGGAGATTTTGG | |
| | TGTTTATTATTGTCAGCAGTATGAATTTTTTGGCC | |
| | AGGGGACCAAGGTCCAGGTCGACATCAAACGTACG | |
| | GTGGCTGCACCATCTGTCTTCATCTTCCCGCCATC | |
| | TGATGAGCAGTTGAAATCTGGAACTGCCTCTGTTG | |
| | TGTGCCTGCTGAATAACTTCTATCCCAGAGAGGCC | |
| | AAAGTACAGTGGAAGGTGGATAACGCCCTCCAATC | |
| | GGGTAACTCCCAGGAGAGTGTCACAGAGCAGGACA | |
| | GCAAGGACAGCACCTACAGCCTCAGCAGCACCCTG | |
| | ACGCTGAGCAAAGCAGACTACGAGAAACACAAAGT | |
| | CTACGCCTGCGAAGTCACCCATCAGGGCCTGAGCT | |
| | CGCCCGTCACAAAGAGCTTCAACAGGGGAGAGTGT | |
| 109 | GAAATTGTGTTGACACAGTCTCCAGCCACCCTGTC | SAP8 LC |
| | TTTGTCTCCAGGGGAAACAGCCATCATCTCTTGTC | (NIH45-46 |
| | GGACCAGTGATCATGGTTCCTTAGCCTGGTATCAA | LC with |
| | CAGAGGCCCGGCCAGGCCCCCAGGCTCGTCATCTA | Q27D, |
| | TTCGGGTTCTACTCGGGCCGCTGGCATCCCAGACA | S28H) |
| | GGTTCAGCGGCAGTCGGTGGGGGGCAGACTACAAT | |
| | CTCAGCATCAGCAACCTGGAGTCGGGAGATTTTGG | |
| | TGTTTATTATTGTCAGCAGTATGAATTTTTTGGCC | |
| | AGGGGACCAAGGTCCAGGTCGACATCAAACGTACG | |
| | GTGGCTGCACCATCTGTCTTCATCTTCCCGCCATC | |
| | TGATGAGCAGTTGAAATCTGGAACTGCCTCTGTTG | |
| | TGTGCCTGCTGAATAACTTCTATCCCAGAGAGGCC | |
| | AAAGTACAGTGGAAGGTGGATAACGCCCTCCAATC | |
| | GGGTAACTCCCAGGAGAGTGTCACAGAGCAGGACA | |
| | GCAAGGACAGCACCTACAGCCTCAGCAGCACCCTG | |
| | ACGCTGAGCAAAGCAGACTACGAGAAACACAAAGT | |
| | CTACGCCTGCGAAGTCACCCATCAGGGCCTGAGCT | |
| | CGCCCGTCACAAAGAGCTTCAACAGGGGAGAGTGT | |

In some embodiments, the antibody or the antigen-binding fragment thereof further comprises a variant Fc constant region. In some embodiments, the antibody is a monoclonal antibody. In some embodiments, the antibody is a chimeric antibody, a humanized antibody, or a humanized monoclonal antibody, the antibody is a single-chain antibody, monovalent antibody, single-chain variable fragment (scFv), scFv-Fc fusion, minibody, Fab, or F(ab′)₂, fragment.

In some embodiments, the antibody or antigen-binding fragment thereof can be detectably labeled or conjugated to a toxin, a therapeutic agent, a polymer (e.g., polyethylene glycol (PEG)), a receptor, an enzyme or a receptor ligand. For example, an antibody of the present invention may be coupled to a toxin (e.g., a tetanus toxin).

In another example, an antibody of the present invention may be coupled to a detectable tag. Such antibodies may be used within diagnostic assays to determine if an animal, such as a human, is infected with HIV-1. Examples of detectable tags include: fluorescent proteins (i.e., green fluorescent protein, red fluorescent protein, yellow fluorescent protein), fluorescent markers (i.e., fluorescein isothiocyanate, rhodamine, texas red), radiolabels (i.e., 3H, 32P, 125I), enzymes (i.e., β-galactosidase, horseradish peroxidase, β-glucuronidase, alkaline phosphatase), or an affinity tag (i.e., avidin, biotin, streptavidin, Fc). Methods to couple antibodies to a detectable tag are known in the art. Harlow et al., Antibodies: A Laboratory Manual, page 319 (Cold Spring Harbor Pub. 1988).

b. Fragment

In some embodiments, an antibody provided herein is an antibody fragment. Antibody fragments include, but are not limited to, Fab, Fab′, Fab′-SH, F(ab′) 2, Fv, and single-chain Fv (scFv) fragments, and other fragments described below, e.g., diabodies, triabodies, tetrabodies, and single-domain antibodies. For a review of certain antibody fragments, see Hudson et al., Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (DOMANTIS, Inc., Waltham, Mass.; see, e.g., U.S. Pat. No. 6,248,516).

Antibody fragments can be made by various techniques, including but not limited to protcolytic digestion of an intact antibody as well as production by recombinant host cells (e.g., E. coli or phage), as described herein.

c. Chimeric and Humanized Antibodies

In some embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

d. Human Antibodies

In some embodiments, an antibody provided herein is a human antibody. Human antibodies can be produced using various techniques known in the art or using techniques described herein. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE technology; U.S. Pat. No. 5,770,429 describing HUMAB technology; U.S. Pat. No. 7,041,870 describing K-M MOUSE technology, and U.S. Patent Application Publication No. US 2007/0061900, describing VELOCIMOUSE technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (Sec, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixuc, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in the McCafferty et al., Nature 348:552-554; Clackson et al., Nature 352:624-628 (1991); Marks et al., J. Mol. Biol. 222:581-597 (1992); Marks and Bradbury, in Methods in Molecular Biology 248:161-175 (Lo, ed., Human Press, Totowa, N.J., 2003); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12:433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12:725-734 (1993). Finally, naive libraries can also be made synthetically by cloning unrearranged V-gene segments from stem cells and using PCR primers containing random sequence to encode the highly variable CDR3 regions and to accomplish rearrangement in vitro, as described by Hoogenboom and Winter, J. Mol. Biol., 227:381-388 (1992). Patent publications describing human antibody phage libraries include, for example, U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered human antibodies or human antibody fragments herein.

e. Variants

In some embodiments, amino acid sequence variants of the antibodies provided herein are contemplated. For example, it may be desirable to improve the binding affinity and/or other biological properties (e.g., reduced polyreactivity, increased half-life, etc.) of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

### Substitution, Insertion, and Deletion Variants

In some embodiments, antibody variants having one or more amino acid substitutions are provided. Sites of interest for substitutional mutagenesis include the HVRs and FRs. Conservative substitutions are defined herein. Amino acid substitutions may be introduced into an antibody of interest and the products screened for a desired activity, e.g., retained/improved antigen binding, decreased immunogenicity, or improved antibody-dependent cell-mediated cytotoxicity (ADCC) and complement-dependent cytotoxicity (CDC).

Accordingly, an antibody of the invention can comprise one or more conservative modifications of the CDRs, heavy chain variable region, or light variable regions described herein. A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof. It substantially retains the activity of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent. Accordingly, within the scope of this invention are heavy chain variable region or light variable regions having one or more point mutations, insertions, deletions, truncations, a fusion protein, or a combination thereof, as well as antibodies having the variant regions.

As used herein, the percent homology between two amino acid sequences is equivalent to the percent identity between the two sequences. The percent identity between the two sequences is a function of the number of identical positions shared by the sequences (i.e., % homology= # of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. (17): 3389-3402. When utilizing BLAST and Gapped BLAST programs, the default parameters of the respective programs (e.g., XBLAST and NBLAST) can be used. (See www.ncbi.nlm.nih.gov).

As used herein, the terms “conservative modifications” or “conservative substitutions” refers to amino acid modifications that do not significantly affect or alter the binding characteristics of the antibody containing the amino acid sequence. Such conservative modifications include amino acid substitutions, additions, and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: (i) amino acids with basic side chains (e.g., lysine, arginine, histidine), (ii) acidic side chains (e.g., aspartic acid, glutamic acid), (iii) uncharged polar side chains (e.g., glycine, asparagine, glutamine, serine, threonine, tyrosine, cysteine, tryptophan), (iv) nonpolar side chains (e.g., alanine, valine, leucine, isoleucine, proline, phenylalanine, methionine), (v) beta-branched side chains (e.g., threonine, valine, isoleucine), and (vi) aromatic side chains (e.g., tyrosine, phenylalanine, tryptophan, histidine).

Alternatively, conservative amino acids can be grouped as described in Lehninger, [Biochemistry, Second Edition; Worth Publishers, Inc. NY, N.Y. (1975), pp. 71-77] as set out below:

CONSERVATIVE SUBSTITUTION

| Side chain characteristic | Amino acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

As still another alternative, exemplary conservative substitutions are set out below:

CONSERVATIVE SUBSTITUTIONS II

| Original residue | Exemplary substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

A conservative substitution of an existing substitution refers to a conservative substitution of the substituting residue. For example, a conservative substitution of Trp at residue G54 (or position 54) refers to a conservative substitution, i.e., Tyr (Y), of Trp at position 54. Likewise, a conservative substitution of Tyr at residue S30 (or position 30) refers to a conservative substitution, i.e., Trp, Phe, Thr, Ser, of Tyr at position 30.

Non-conservative substitutions will entail exchanging a member of one of these classes for another class.

An exemplary substitutional variant is an affinity matured antibody, which may be conveniently generated, e.g., using phage display-based affinity maturation techniques such as those described in, e.g., Hoogenboom et al., in Methods in Molecular Biology 178:1-37 (O'Brien et al., ed., Human Press, Totowa, N.J., (2001). Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites are created or removed.

For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Glycosylation of the constant region on N297 may be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyltransferase, such that antibodies expressed in such a cell line exhibit hypofucosylation. PCT Publication WO 03/035835 by Presta describes a variant Chinese Hamster Ovary cell line, Led 3 cells, with reduced ability to attach fucose to Asn (297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyltransferases (e.g., beta (1,4)-N-acetylglucosaminyltransferase III (GnTIII)) such that antibodies expressed in the engineered cell lines exhibit increased bisecting GlcNac structures which result in increased ADCC activity of the antibodies (see also Umana et al. (1999) Nat. Biotech. 17:176-180).

### Fc Region Variants

The variable regions of the antibody described herein can be linked (e.g., covalently linked or fused) to an Fc, e.g., an IgG1, IgG2, IgG3 or IgG4 Fc, which may be of any allotype or isoallotype, e.g., for IgG1:Glm, Glml(a), Glm2(x), Glm3(f), Glml7(z); for IgG2: G2m, G2m23 (n); for IgG3: G3m, G3m21(gl), G3m28(g5), G3ml1(b0), G3m5(bl), G3ml3(b3), G3ml4(b4), G3ml0(b5), G3ml5(s), G3ml6(t), G3m6(c3), G3m24(c5), G3m26(u), G3m27(v); and for K:Km, Km1, Km2, Km3 (see, e.g., Jefferies et al. (2009) mAbs 1:1). In some embodiments, the antibodies variable regions described herein are linked to an Fc that binds to one or more activating Fc receptors (FcγI, FcγIIa or FcγIIIa), and thereby stimulate ADCC and may cause T cell depletion. In some embodiments, the antibody variable regions described herein are linked to an Fc that causes depletion.

In some embodiments, the antibody variable regions described herein may be linked to an Fc comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody. The numbering of residues in the Fc region is that of the EU index of Kabat.

The Fc region encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes such as IgA, IgD, IgE and IgM, The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination. In some embodiments, an antibody of this invention has an Fc region other than that of a wild type IgAQ1. The antibody can have an Fc region from that of IgG (e.g., IgG1, IgG2, IgG3, and IgG4) or other classes such as IgA2, IgD, IgE, and IgM. The Fc can be a mutant form of IgAQ1.

The constant region of an immunoglobulin is responsible for many important antibody functions, including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgG1, IgG2, IgG3, and IgG4.

Ig molecules interact with multiple classes of cellular receptors. For example, IgG molecules interact with three classes of Fcγ receptors (FcγR) specific for the IgG class of antibody, namely FcγRI, FcγRII, and FcγRIIL. The important sequences for the binding of IgG to the FcγR receptors have been reported to be located in the CH2 and CH3 domains. The serum half-life of an antibody is influenced by the ability of that antibody to bind to an FcR.

In some embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased ADCC, (b) increased or decreased CDC, (c) has increased or decreased affinity for C1q and/or (d) has increased or decreased affinity for an Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc. substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli such as proline iminopeptidase. In other embodiments, one or more glycosylation sites within the Fc domain may be removed. Residues that are typically glycosylated (e.g., asparagine) may confer cytolytic response. Such residues may be deleted or substituted with unglycosylated residues (e.g., alanine). In other embodiments, sites involved in interaction with complement, such as the C1q binding site, may be removed from the Fc region. For example, one may delete or substitute the EKK sequence of human IgG1. In some embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1. Specific examples of variant Fc domains are disclosed, for example, in WO 97/34631 and WO 96/32478.

In one embodiment, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished CDC. This approach is described in further detail in U.S. Pat. Nos. 6,194,551 by Idusogic et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region may be modified to increase ADCC and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 305I, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Kabat (WO00/42072).

Other Fc modifications that can be made to Fes are those for reducing or ablating binding to FcγR and/or complement proteins, thereby reducing or ablating Fc-mediated effector functions such as ADCC, antibody-dependent cellular phagocytosis (ADCP), and CDC. Exemplary modifications include but are not limited to substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; WO00/42072; WO01/58957; WO02/06919; WO04/016750; WO04/029207; WO04/035752; WO04/074455; WO04/099249; WO04/063351; WO05/070963; WO05/040217, WO05/092925 and WO06/020114).

Fc variants that enhance affinity for an inhibitory receptor FcγRIIb may also be used. Such variants may provide an Fc fusion protein with immune-modulatory activities related to FcγRIIb cells, including, for example, B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art including but not limited to, equilibrium methods (e.g., ELISA, or radioimmunoassay), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., cd., Fundamental Immunology, 4th Ed., Lippincott-Raven, Philadelphia (1999), which focuses on antibody-immunogen interactions.

In some embodiments, the antibody is modified to increase its biological half-life. Various approaches are possible. For example, this may be done by increasing the binding affinity of the Fc region for FcRn. For example, one or more of the following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CHI or CL region to contain a salvage 20) receptor binding epitope taken from two loops of a CH2 domain of an Fc region of an IgG, as described in U.S. Pat. Nos. 5,869,046 and 6, 121,022 by Presta et al. Other exemplary variants that increase binding to FcRn and/or improve pharmacokinetic properties include substitutions at positions 259, 308, 428, and 434, including for example 2591, 308F, 428L, 428M, 434S, 434H, 434F, 434Y, and 434M. Other variants that increase Fc binding to FcRn include: 250E, 250Q, 25 428L, 428F, 250Q/428L (Hinton et al,, 2004, J. Biol. Chem. 279 (8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276 (9): 6591-6604), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In some embodiments, hybrid IgG isotypes with particular biological characteristics may be used. For example, an IgG1/IgG3 hybrid variant may be constructed by substituting IgG 1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 4221, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed chat comprises one or more substitutions, e.g., one or more of the following amino acid substitutions: 233E, 234L, 235L, 236G (referring to an insertion of a glycine at position 236), and 321 h.

Moreover, the binding sites on human IgG1 for FcγRI, FcγRII, FcγRIII, and FcRn have been mapped and variants with improved binding have been described (see Shields, R. L. et al. (2001) J. Biol. Chem. 276:6591-6604). Specific mutations at positions 256, 290, 298, 333, 334, and 339 were shown to improve binding to FcγRIII. Additionally, the following combination mutants were shown to improve FcγRIII binding: T256A/S298A, S298A/E333A, S298A/K224A, and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/1332E and S239D/1332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/1332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/1332E, S239D/1332E/A330L, L235V/F243L/R292P/Y300L/P396L, and M428L/N434S.

In some embodiments, an Fc is chosen that has reduced binding to FcγRs. An exemplary Fc, e.g., IgG1 Fc, with reduced FcγR binding, comprises the following three amino acid substitutions: L234A, L235E, and G237A.

In some embodiments, an Fc is chosen that has reduced complement fixation. An exemplary Fc, e.g., IgG1 Fc, with reduced complement fixation, has the following two amino acid substitutions: A330S and P331S.

In some embodiments, an Fc is chosen that has essentially no effector function, i.e., it has reduced binding to FcγRs and reduced complement fixation. An exemplary Fc, e.g., IgG1 Fc, that is effectorless, comprises the following five mutations: L234A, L235E, G237A, A330S, and P331S.

When using an IgG4 constant domain, it is usually preferable to include the substitution S228P, which mimics the hinge sequence in IgG1 and thereby stabilizes IgG4 molecules.

Fc Mutations that Increase Serum Half-Life

In some embodiments, the Fc region or Fc domain of the anti-HIV gp120 directed antibody comprise amino acid modifications that promote an increased serum half-life of the anti-binding molecule. Mutations that increase the half-life of an antibody have been described. In one embodiment, the Fc region or Fc domain of one or both of the CD3-targeting heavy chain and the HIV antigen-targeting heavy chain comprise a methionine to tyrosine substitution at position 252 (EU numbering), a serine to threonine substitution at position 254 (EU numbering), and a threonine to glutamic acid substitution at position 256 (EU numbering). Sec, e.g., U.S. Pat. No. 7,658,921. This type of mutant, designated as a “YTE mutant” exhibits a four-fold increased half-life relative to wild-type versions of the same antibody (Dall'Acqua, et al., J Biol Chem, 281:23514-24 (2006); Robbie, et al., Antimicrob Agents Chemotherap., 57 (12): 6147-6153 (2013)). In certain embodiments, the Fc region or Fc domain of one or both of the CD3-targeting heavy chain and the HIV antigen-targeting heavy chain comprise an IgG constant domain comprising one, two, three or more amino acid substitutions of amino acid residues at positions 251-257, 285-290, 308-314, 385-389, and 428-436 (EU numbering). Alternatively, M428L and N434S (“LS”) substitutions can increase the pharmacokinetic half-life of the multi-specific antigen binding molecule. In other embodiments, the Fc region or Fc domain of one or both of the CD3-targeting heavy chain and the HIV antigen-targeting heavy chain comprise a M428L and N434S substitution (EU numbering). In other embodiments, the Fc region or Fc domain of one or both of the CD3-targeting heavy chain and the HIV antigen-targeting heavy chain comprise T250Q and M428L (EU numbering) mutations. In other embodiments, the Fc region or Fc domain of one or both of the CD3-targeting heavy chain and the HIV antigen-targeting heavy chain comprise H433K and N434F (EU numbering) mutations.

Fc Mutations that Enhance Effector Activity

In some embodiments, the Fc region or Fc domain of the anti-HIV gp120 directed antibody comprise post-translational and/or amino acid modifications that increase effector activity, e.g., have improved FcγIIIa binding and increased antibody-dependent cellular cytotoxicity (ADCC). In some embodiments, the Fc region or Fc domain of the anti-HIV gp120 directed antibody comprises DE modifications (i.e., S239D and 1332E by EU numbering) in the Fc region. In some embodiments, the Fc region or Fc domain of the anti-HIV gp120 directed antibody comprises DEL modifications (i.e., S239D, 1332E and A330L by EU numbering) in the Fc region. In some embodiments, the Fc region or Fc domain of the anti-HIV gp 120 directed antibody comprises DEA modifications (i.e., S239D, 1332E and G236A by EU numbering) in the Fc region. In some embodiments, the Fc region or Fc domain of the anti-HIV gp120 directed antibody comprises DEAL modifications (i.e., S239D, 1332E, G236A and A330L by EU numbering) in the Fc region. See, e.g., U.S. Pat. Nos. 7,317,091; 7,662,925; 8,039,592; 8,093,357; 8,093,359; 8,383,109; 8,388,955; 8,735,545; 8,858,937; 8,937,158; 9,040,041; 9,353,187; 10,184,000; and 10,584,176. Additional amino acid modifications that increase effector activity, e.g., have improved FcγIIIa binding and increased antibody-dependent cellular cytotoxicity (ADCC) include without limitation (EU numbering) F243L/R292P/Y300L/V305I/P396L; S298A/E333A/K334A; or L234Y/L235Q/G236W/S239M/H268D/D270E/S298A on a first Fc domain and D270E/K326D/A330M/K334E on a second Fc domain. Amino acid mutations that increase C1q binding and complement-dependent cytotoxicity (CDC) include without limitation (EU numbering) S267E/H268F/S324T or K326W/E333S. Fc region mutations that enhance effector activity are reviewed in, e.g., Wang, et al., Protein Cell (2018) 9 (1): 63-73; and Saunders, Front Immunol. (2019) 10:1296.

In other embodiments, the anti-HIV gp120 directed antibody or antigen-binding fragment thereof has modified glycosylation, which, e.g., may be introduced post-translationally or through genetic engineering. In some embodiments, the anti-HIV gp120 directed antibody or antigen-binding fragment thereof is afucosylated, e.g., at a glycosylation site present in the antibody or antigen-binding fragment thereof. Most approved monoclonal antibodies are of the IgG1 isotype, where two N-linked biantennary complex-type oligosaccharides are bound to the Fc region. The Fc region exercises the effector function of ADCC through its interaction with leukocyte receptors of the FcγR family. Afucosylated monoclonal antibodies are monoclonal antibodies engineered so that the oligosaccharides in the Fc region of the antibody do not have any fucose sugar units.

f. Multivalent Antibodies

In one embodiment, the antibodies of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105. In each case, at least one of the binding sites will comprise an epitope, motif or domain associated with a DLL3 isoform.

In one embodiment, the antibodies are bispecific antibodies in which the two chains have different specificities, as described in Millstein et al., 1983, Nature, 305:537-539. Other embodiments include antibodies with additional specificities such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

As stated above, multivalent antibodies may immunospecifically bind to different epitopes of the desired target molecule or may immunospecifically bind to both the target molecule as well as a heterologous epitope, such as a heterologous polypeptide or solid support material. In some embodiments, the multivalent antibodies may include bispecific antibodies or trispecific antibodies.

Bispecific antibodies are antibodies that have binding specificities for at least two different epitopes. Exemplary bispecific antibodies can bind to two different epitopes of a single antigen. Other such antibodies can combine a first antigen binding site with a binding site for a second antigen. Alternatively, an anti-HIV arm can be combined with an arm that binds to a triggering molecule on a leukocyte, such as a T-cell receptor molecule (for example, CD3), or Fc receptors for IgG (Fc gamma R), such as Fc gamma RI (CD64), Fc gamma RII (CD32) and Fc gamma RIII (CD16), so as to focus and localize cellular defense mechanisms to the infected cell. Bispecific antibodies also can be used to localize cytotoxic agents to infected cells. Bispecific antibodies can be prepared as full-length antibodies or antibody fragments (for example, F(ab′)2 bispecific antibodies). For example, WO 96/16673 describes a bispecific anti-ErbB2/anti-Fc gamma RIII antibody and U.S. Pat. No. 5,837,234 discloses a bispecific anti-ErbB2/anti-Fc gamma RI antibody. For example, a bispecific anti-ErbB2/Fc alpha antibody is reported in WO98/02463; U.S. Pat. No. 5,821,337 teaches a bispecific anti-ErbB2/anti-CD3 antibody. See also, for example, Mouquet et al., Polyreactivity Increases The Apparent Affinity Of Anti-HIV Antibodies By Heteroligation. NATURE. 467, 591-5 (2010).

Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art and are disclosed in U.S. Pat. No. 4,676,980, along with a number of cross-linking techniques.

Methods for making bispecific antibodies are known in the art. Traditional production of full length bispecific antibodies is based on the co-expression of two immunoglobulin heavy chain-light chain pairs, where the two chains have different specificities (see, for example, Millstein et al., Nature, 305:537-539 (1983)). Similar procedures are disclosed in, for example, WO 93/08829, Traunecker et al., EMBO J., 10:3655-3659 (1991) and see also; Mouquet et al., Polyreactivity Increases The Apparent Affinity Of Anti-HIV Antibodies By Heteroligation. NATURE. 467, 591-(2010).

Alternatively, antibody variable regions with the desired binding specificities (antibody-antigen combining sites) are fused to immunoglobulin constant domain sequences. The fusion is with an Ig heavy chain constant domain, comprising at least part of the hinge, CH2, and CH3 regions. According to some embodiments, the first heavy-chain constant region (CH1) containing the site necessary for light chain bonding, is present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

Techniques for generating bispecific antibodies from antibody fragments also have been described in the literature. For example, bispecific antibodies can be prepared using chemical linkage. For example, Brennan et al., Science, 229:81 (1985) describe a procedure wherein intact antibodies are proteolytically cleaved to generate F(ab′) 2 fragments. These fragments are reduced in the presence of the dithiol complexing agent, sodium arsenite, to stabilize vicinal dithiols and prevent intermolecular disulfide formation. The Fab′ fragments generated then are converted to thionitrobenzoate (TNB) derivatives. One of the Fab′-TNB derivatives then is reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Other modifications of the antibody are contemplated herein. For example, the antibody can be linked to one of a variety of nonproteinaceous polymers, for example, polyethylene glycol, polypropylene glycol, polyoxyalkylenes, or copolymers of polyethylene glycol and polypropylene glycol. The antibody also can be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are disclosed in, for example, Remington's Pharmaceutical Sciences, 16th edition, Oslo, A., Ed., (1980).

In some embodiments, a bispecific antibody of the present invention comprising: a first antigen binding arm binding to a first antigen (e.g., gp120) and a second antigen binding arm binding to a second antigen, wherein the first antigen and the second antigen are different.

In some embodiments, the first antigen binding arm comprising: (a) the VH CDRs 1-3 and the VL CDRs 1-3, respectively set forth in SEQ ID NOs: 11-16; or (b) the HC and the LC respectively set forth in SEQ ID NOs: 3 and 2; SEQ ID NOs: 3 and 6; SEQ ID NOs: 3 and 7; SEQ ID NOs: 3 and 8; SEQ ID NOs: 3 and 9; SEQ ID NOs: 3 and 10; SEQ ID NOs: 1 and 5; SEQ ID NOs: 1 and 8; or SEQ ID NOs: 1 and 9.

In some embodiments, the second antigen is selected from the group consisting of CD3, FcγRI (CD64), FcγRII (CD32), FcγRIII (CD 16); CD89, CCR5, CD4, CD8, CD28, CD137, CTLA-4, gp41, killer cell immunoglobulin like receptor, three Ig domains and long cytoplasmic tail 1 (KIR3DL1), killer cell immunoglobulin like receptor, three Ig domains and long cytoplasmic tail 1 (KIR3DL1), killer cell immunoglobulin like receptor, two Ig domains and long cytoplasmic tail 1 (KIR2DL1), killer cell immunoglobulin like receptor, two Ig domains and long cytoplasmic tail 2 (KIR2DL2), killer cell immunoglobulin like receptor, two Ig domains and long cytoplasmic tail 3 (KIR2DL3), killer cell lectin like receptor C1 (KLRC1), killer cell lectin like receptor C2 (KLRC2), killer cell lectin like receptor C3 (KLRC3), killer cell lectin like receptor C4 (KLRC4), killer cell lectin like receptor D1 (KLRD1), killer cell lectin like receptor K1 (KLRK1), natural cytotoxicity triggering receptor 3 (NCR3 or NKp30), natural cytotoxicity triggering receptor 2 (NCR2 or NK-p44), natural cytotoxicity triggering receptor 1 (NCR1 or NK-p46), CD226 (DNAM-1), cytotoxic and regulatory T cell molecule (CRTAM or CD355), signaling lymphocytic activation molecule family member 1 (SLAMF1), CD48 (SLAMF2), lymphocyte antigen 9 (LY9 or SLAMF3), CD244 (2B4 or SLAMF4), CD84 (SLAMF5), SLAM family member 6 (SLAMF6 or NTB-A), SLAM family member 7 (SLAMF7 or CRACC), CD27 (TNFRSF7), semaphorin 4D (SEMA4D or CD 100), CD 160 (NK1), an immune checkpoint protein (e.g., PD-1, PD-L1, CTLA-4), and a second epitope of gp120.

g. Antibody Derivatives

An antibody provided herein may be further modified to contain additional nonproteinaceous moieties that are known in the art and readily available. The moieties suitable for derivatization of the antibody include but are not limited to water-soluble polymers.

Non-limiting examples of water-soluble polymers include, but are not limited to, PEG, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaccous moiety that may be selectively heated by exposure to radiation are provided. In one embodiment, the nonproteinaccous moiety is a carbon nanotube (Kam et al., Proc. Natl. Acad. Sci. USA 102:11600-11605 (2005)). The radiation may be of any wavelength, and includes, but is not limited to, wavelengths that do not harm ordinary cells, but which heat the nonproteinaccous moiety to a temperature at which cells proximal to the antibody-nonproteinaceous moiety are killed.

Another modification of the antibodies described herein is pegylation. An antibody can be pegylated to, for example, increase the biological (e.g., serum) half-life of the antibody. To pegylate an antibody, the antibody, or fragment thereof, typically is reacted with PEG, such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In some embodiments, the antibody to be pegylated is an aglycosylated antibody. Methods for pegylating proteins are known in the art and can be applied to the antibodies described herein. See, for example, EP 0 154 316 by Nishimura et al. and EP0401384 by Ishikawa et al.

The present invention also encompasses a human monoclonal antibody described herein conjugated to a therapeutic agent, a polymer, a detectable label or enzyme. In one embodiment, the therapeutic agent is a cytotoxic agent. In one embodiment, the polymer is PEG.

h. Nucleic Acids, Expression Cassettes, and Vectors

The present invention provides isolated nucleic acid segments that encode the polypeptides, peptide fragments, and coupled proteins of the invention. The nucleic acid segments of the invention also include segments that encode for the same amino acids due to the degeneracy of the genetic code. For example, the amino acid threonine is encoded by ACU, ACC, ACA, and ACG and is therefore degenerate. It is intended that the invention includes all variations of the polynucleotide segments that encode for the same amino acids. Such mutations are known in the art (Watson et al., Molecular Biology of the Gene, Benjamin Cummings 1987). Mutations also include alteration of a nucleic acid segment to encode for conservative amino acid changes, for example, the substitution of leucine for isoleucine and so forth. Such mutations are also known in the art. Thus, the genes and nucleotide sequences of the invention include both the naturally occurring sequences as well as mutant forms.

The nucleic acid segments of the invention may be contained within a vector. A vector may include, but is not limited to, any plasmid, phagemid, F-factor, virus, cosmid, or phage in a double- or single-stranded linear or circular form which may or may not be self transmissible or mobilizable. The vector can also transform a prokaryotic or eukaryotic host either by integration into the cellular genome or exist extra-chromosomally (e.g., autonomous replicating plasmid with an origin of replication).

Preferably the nucleic acid segment in the vector is under the control of, and operably linked to, an appropriate promoter or other regulatory elements for transcription in vitro or in a host cell, such as a eukaryotic cell, or a microbe, e.g., bacteria. The vector may be a shuttle vector that functions in multiple hosts. The vector may also be a cloning vector that typically contains one or a small number of restriction endonuclease recognition sites at which foreign DNA sequences can be inserted in a determinable fashion. Such insertion can occur without loss of essential biological function of the cloning vector. A cloning vector may also contain a marker gene that is suitable for use in the identification and selection of cells transformed with the cloning vector. Examples of marker genes are tetracycline resistance or ampicillin resistance. Many cloning vectors are commercially available (Stratagene, New England Biolabs, Clonetech).

The nucleic acid segments of the invention may also be inserted into an expression vector. Typically an expression vector contains prokaryotic DNA elements coding for a bacterial replication origin and an antibiotic resistance gene to provide for the amplification and selection of the expression vector in a bacterial host; regulatory elements that control initiation of transcription such as a promoter; and DNA elements that control the processing of transcripts such as introns, or a transcription termination/polyadenylation sequence.

Methods to introduce nucleic acid segment into a vector are available in the art (Sambrook et al., Molecular Cloning: A Laboratory Manual, 3rd edition, Cold Spring Harbor Press, Cold Spring Harbor, N.Y. (2001)). Briefly, a vector into which a nucleic acid segment is to be inserted is treated with one or more restriction enzymes (restriction endonuclease) to produce a linearized vector having a blunt end, a “sticky” end with a 5′ or a 3′ overhang, or any combination of the above. The vector may also be treated with a restriction enzyme and subsequently treated with another modifying enzyme, such as a polymerase, an exonuclease, a phosphatase or a kinase, to create a linearized vector that has characteristics useful for ligation of a nucleic acid segment into the vector. The nucleic acid segment that is to be inserted into the vector is treated with one or more restriction enzymes to create a linearized segment having a blunt end, a “sticky” end with a 5′ or a 3′ overhang, or any combination of the above. The nucleic acid segment may also be treated with a restriction enzyme and subsequently treated with another DNA modifying enzyme. Such DNA modifying enzymes include, but are not limited to, polymerase, exonuclease, phosphatase or a kinase, to create a nucleic acid segment that has characteristics useful for ligation of a nucleic acid segment into the vector.

The treated vector and nucleic acid segment are then ligated together to form a construct containing a nucleic acid segment according to methods available in the art (Sambrook et al., Molecular Cloning: A Laboratory Manual, 3rd edition, Cold Spring Harbor Press, Cold Spring Harbor, N.Y. (2001)). Briefly, the treated nucleic acid fragment, and the treated vector are combined in the presence of a suitable buffer and ligase. The mixture is then incubated under appropriate conditions to allow the ligase to ligate the nucleic acid fragment into the vector.

The invention also provides an expression cassette which contains a nucleic acid sequence capable of directing expression of a particular nucleic acid segment of the invention, either in vitro or in a host cell. Also, a nucleic acid segment of the invention may be inserted into the expression cassette such that an anti-sense message is produced. The expression cassette is an isolatable unit such that the expression cassette may be in linear form and functional for in vitro transcription and translation assays. The materials and procedures to conduct these assays are commercially available from Promega Corp. (Madison, Wis.). For example, an in vitro transcript may be produced by placing a nucleic acid sequence under the control of a T7 promoter and then using T7 RNA polymerase to produce an in vitro transcript. This transcript may then be translated in vitro through use of a rabbit reticulocyte lysate. Alternatively, the expression cassette can be incorporated into a vector allowing for replication and amplification of the expression cassette within a host cell or also in vitro transcription and translation of a nucleic acid segment.

Such an expression cassette may contain one or a plurality of restriction sites allowing for placement of the nucleic acid segment under the regulation of a regulatory sequence. The expression cassette can also contain a termination signal operably linked to the nucleic acid segment as well as regulatory sequences required for proper translation of the nucleic acid segment. The expression cassette containing the nucleic acid segment may be chimeric, meaning that at least one of its components is heterologous with respect to at least one of its other components. The expression cassette may also be one that is naturally occurring but has been obtained in a recombinant form useful for heterologous expression. Expression of the nucleic acid segment in the expression cassette may be under the control of a constitutive promoter or an inducible promoter, which initiates transcription only when the host cell is exposed to some particular external stimulus.

The expression cassette may include in the 5′-3′ direction of transcription, a transcriptional and translational initiation region, a nucleic acid segment and a transcriptional and translational termination region functional in vivo and/or in vitro. The termination region may be native with the transcriptional initiation region, may be native with the nucleic acid segment, or may be derived from another source.

The regulatory sequence can be a polynucleotide sequence located upstream (5′ non-coding sequences), within, or downstream (3′ non-coding sequences) of a coding sequence, and which influences the transcription, RNA processing or stability, or translation of the associated coding sequence. Regulatory sequences can include, but are not limited to, enhancers, promoters, repressor binding sites, translation leader sequences, introns, and polyadenylation signal sequences. They may include natural and synthetic sequences as well as sequences, which may be a combination of synthetic and natural sequences. While regulatory sequences are not limited to promoters, some useful regulatory sequences include constitutive promoters, inducible promoters, regulated promoters, tissue-specific promoters, viral promoters, and synthetic promoters.

A promoter is a nucleotide sequence that controls the expression of the coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA-box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may contain DNA sequences that are involved in the binding of protein factors that control the effectiveness of transcription initiation in response to physiological or developmental conditions.

The invention also provides a construct containing a vector and an expression cassette. The vector may be selected from, but not limited to, any vector previously described. Into this vector may be inserted an expression cassette through methods known in the art and previously described (Sambrook et al., Molecular Cloning: A Laboratory Manual, 3rd edition, Cold Spring Harbor Press, Cold Spring Harbor, N.Y. (2001)). In one embodiment, the regulatory sequences of the expression cassette may be derived from a source other than the vector into which the expression cassette is inserted. In another embodiment, a construct containing a vector and an expression cassette is formed upon insertion of a nucleic acid segment of the invention into a vector that itself contains regulatory sequences. Thus, an expression cassette is formed upon insertion of the nucleic acid segment into the vector. Vectors containing regulatory sequences are available commercially, and methods for their use are known in the art (Clonetech, Promega, Stratagene).

In another aspect, this disclosure also provides (i) a nucleic acid molecule encoding a polypeptide chain of the antibody or antigen-binding fragment thereof described above; (ii) a vector comprising the nucleic acid molecule as described; and (iii) a cultured host cell comprising the vector as described. Also provided is a method for producing a polypeptide, comprising: (a) obtaining the cultured host cell as described; (b) culturing the cultured host cell in a medium under conditions permitting expression of a polypeptide encoded by the vector and assembling of an antibody or fragment thereof; and (c) purifying the antibody or fragment from the cultured cell or the medium of the cell.

i. Methods of Production

Antibodies may be produced using recombinant methods and compositions, e.g., as described in U.S. Pat. No. 4,816,567. In one embodiment, an isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, a nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, cd., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified, which may be used in conjunction with insect cells, particularly for transfection of Spodoptera frugiperda cells.

Plant cell cultures can also be utilized as hosts. Sec, e.g., U.S. Pat. Nos. 5,959,177, 6,040,498, 6,420,548, 7,125,978, and 6,417,429 (describing PLANTIBODIES technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include CHO cells, including DHFR-CHO cells (Urlaub et al., Proc. Natl. Acad. Sci. USA 77:4216 (1980)); and myeloma cell lines such as Y0, NS0, and Sp2/0. For a review of certain mammalian host cell lines suitable for antibody production, see, e.g., Yazaki and Wu, Methods in Molecular Biology, Vol. 248 (B. K. C. Lo, cd., Humana Press, Totowa, N.J.), pp. 255-268 (2003).

### B. Compositions and Formulations

In another aspect, this disclosure also provides a pharmaceutical composition comprising (i) at least one anti-HIV antibody, or antigen binding portion thereof, the nucleic acid, or the vector, as described above; and (ii) a pharmaceutically acceptable carrier.

In some embodiments, the pharmaceutical composition further comprises a second therapeutic agent. In some embodiments, the second therapeutic agent comprises an antiviral agent or one or more additional antibodies. In some embodiments, the one or more additional antibodies comprise a second anti-HIV antibody (e.g., an isolated anti-HIV bNAb as disclosed) or antigen binding portion thereof, or a third antibody binding to a third antigen. In some embodiments, the third antigen is selected from the group consisting of CD3, FcγRI (CD64), FcγRII (CD32), FcγRIII (CD 16); CD89, CCR5, CD4, CD8, CD28, CD137, CTLA-4, gp41, killer cell immunoglobulin like receptor, three Ig domains and long cytoplasmic tail 1 (KIR3DL1), killer cell immunoglobulin like receptor, three Ig domains and long cytoplasmic tail 1 (KIR3DL1), killer cell immunoglobulin like receptor, two Ig domains and long cytoplasmic tail 1 (KIR2DL1), killer cell immunoglobulin like receptor, two Ig domains and long cytoplasmic tail 2 (KIR2DL2), killer cell immunoglobulin like receptor, two Ig domains and long cytoplasmic tail 3 (KIR2DL3), killer cell lectin like receptor C1 (KLRC1), killer cell lectin like receptor C2 (KLRC2), killer cell lectin like receptor C3 (KLRC3), killer cell lectin like receptor C4 (KLRC4), killer cell lectin like receptor D1 (KLRD1), killer cell lectin like receptor K1 (KLRK1), natural cytotoxicity triggering receptor 3 (NCR3 or NKp30), natural cytotoxicity triggering receptor 2 (NCR2 or NK-p44), natural cytotoxicity triggering receptor 1 (NCR1 or NK-p46), CD226 (DNAM-1), cytotoxic and regulatory T cell molecule (CRTAM or CD355), signaling lymphocytic activation molecule family member 1 (SLAMF1), CD48 (SLAMF2), lymphocyte antigen 9 (LY9 or SLAMF3), CD244 (2B4 or SLAMF4), CD84 (SLAMF5), SLAM family member 6 (SLAMF6 or NTB-A), SLAM family member 7 (SLAMF7 or CRACC), CD27 (TNFRSF7), semaphorin 4D (SEMA4D or CD 100), CD 160 (NK1), an immune checkpoint protein (e.g., PD-1, PD-L1, CTLA-4), and a second epitope of gp120.

In some embodiments, the antiviral agent is selected from the group consisting of a non-nucleoside reverse transcriptase inhibitor, a protease inhibitor, an entry or fusion inhibitor, and an integrase inhibitor.

The pharmaceutical compositions of the invention also can be administered as a vaccine (e.g., as an AAV-based vaccine) or in a combination therapy with, for example, another immune-stimulatory agent, an antiviral agent, or a vaccine, etc. In some embodiments, a composition comprises an antibody of this invention at a concentration of at least 1 mg/ml, 5 mg/ml, 10 mg/ml, 50 mg/ml, 100 mg/ml, 150 mg/ml, 200 mg/ml, 1-300 mg/ml, or 100-300 mg/ml.

In some embodiments, the second therapeutic agent comprises an anti-inflammatory drug or an antiviral compound. In some embodiments, the antiviral compound comprises: a nucleoside analog, a peptoid, an oligopeptide, a polypeptide, a protease inhibitor, a 3C-like protease inhibitor, a papain-like protease inhibitor, or an inhibitor of an RNA dependent RNA polymerase. In some embodiments, the antiviral compound may include: acyclovir, gancyclovir, vidarabine, foscarnet, cidofovir, amantadine, ribavirin, trifluorothymidine, zidovudine, didanosine, zalcitabine or an interferon. In some embodiments, the interferon is an interferon-a or an interferon-B.

Also within the scope of this disclosure is use of the pharmaceutical composition in the preparation of a medicament for the diagnosis, prophylaxis, treatment, or combination thereof of a condition resulting from HIV-1 infection.

The pharmaceutical composition can comprise any number of excipients. Excipients that can be used include carriers, surface-active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

Preferably, a pharmaceutical composition is suitable for intravenous, intramuscular, subcutaneous, parenteral, spinal or epidermal administration (e.g., by injection or infusion). Depending on the route of administration, the active compound can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the present invention described herein can be administered via a non-parenteral route, such as a topical, epidermal or mucosal route of administration, e.g., intranasally, orally, vaginally, rectally, sublingually or topically.

The pharmaceutical compositions of the invention may be prepared in many forms that include tablets, hard or soft gelatin capsules, aqueous solutions, suspensions, and liposomes and other slow-release formulations, such as shaped polymeric gels. An oral dosage form may be formulated such that the antibody is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

An antibody can be formulated for parenteral administration (e.g., by injection, for example, bolus injection or continuous infusion) and may be presented in unit dosage form in ampules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Pharmaceutical compositions suitable for rectal administration can be prepared as unit dose suppositories. Suitable carriers include saline solution and other materials commonly used in the art.

For administration by inhalation, an antibody can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, an antibody may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, an antibody may be administered via a liquid spray, such as via a plastic bottle atomizer.

Pharmaceutical compositions of the invention may also contain other ingredients such as flavorings, colorings, anti-microbial agents, or preservatives. It will be appreciated that the amount of an antibody required for use in treatment will vary not only with the particular carrier selected but also with the route of administration, the nature of the condition being treated and the age and condition of the patient. Ultimately the attendant health care provider may determine proper dosage. In addition, a pharmaceutical composition may be formulated as a single unit dosage form.

The pharmaceutical composition of the present invention can be in the form of sterile aqueous solutions or dispersions. It can also be formulated in a microemulsion, liposome, or other ordered structure suitable to high drug concentration.

An antibody of the present invention described herein can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably, until the patient shows partial or complete amelioration of symptoms of disease. Thereafter, the patient can be administered a prophylactic regime.

The amount of active ingredient that can be combined with a carrier material to produce a single dosage form will vary depending upon the subject being treated and the particular mode of administration and will generally be that amount of the composition, which produces a therapeutic effect. Generally, out of one hundred percent, this amount will range from about 0.01% to about 99% of active ingredient, preferably from about 0.1% to about 70%, most preferably from about 1% to about 30% of active ingredient in combination with a pharmaceutically acceptable carrier.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Sec, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparati (U.S. Pat. Nos. 4,447,233 and 4,447,224); and (5) osmotic devices (U.S. Pat. Nos. 4,439,196 and 4,475,196); the disclosures of which are incorporated herein by reference.

In some embodiments, the human monoclonal antibodies of the invention described herein can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs. Sec, e.g., U.S. Pat. Nos. 4,522,811; 5,374,548; 5,416,016; and 5,399,331; V.V. Ranade (1989) Clin. Pharmacol. 29:685; Umezawa et al., (1988) Biochem. Biophys. Res. Commun. 153:1038; Bloeman et al. (1995) FEBS Lett. 357:140; M. Owais et al. (1995) Antimicrob. Agents Chemother. 39:180; Briscoe et al. (1995) Am. Physiol. 1233:134; Schreier et al. (1994). Biol. Chem. 269:9090; Keinanen and Laukkanen (1994) FEBS Lett. 346:123; and Killion and Fidler (1994) Immunomethods 4:273.

In some embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor-mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can also be delivered in a vesicle, in particular, a liposome (see, for example, Langer (1990) Science 249:1527-1533).

The use of nanoparticles to deliver the antibodies of the present invention is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389), incorporated herein by reference. Nanoparticles may be developed and conjugated to antibodies contained in pharmaceutical compositions to target cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded. 25

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25TM pen, HUMALOG™ pen, HUMALIN 70/30TM pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.) and the HUMIRA™ Pen (Abbott Labs, Abbott Park, IL), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, it is preferred that the antibody is contained in about 5 to about 300 mg and in about 10 to about 300 mg for the other dosage forms.

### C. Methods and Uses

a. Methods of Treatment

According to another embodiment, the present invention provides a method for treating a mammal infected with a virus (e.g., HIV) infection, comprising administering to the mammal a pharmaceutical composition comprising the anti-HIV antibodies disclosed herein. According to one embodiment, the method for treating a mammal infected with HIV comprises administering to the mammal a pharmaceutical composition that comprises an antibody of the present invention, or a fragment thereof. The compositions of the invention can include more than one antibody having the characteristics disclosed (for example, a plurality or pool of antibodies). It also can include other HIV neutralizing antibodies as are known in the art, for example, but not limited to, GS-9722 (clipovimab), PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03 (all of which bind the V3-glycan region), 2F5, 4E10, M66.6, CAP206-CH12, 10E8, 10E8v4, 10E8-5R-100cF, DH511.11P, 7b2, and LN01 (all of which bind the MPER of gp41); PG9, PG16, CH01-04 (all of which bind VIV2-glycan), 2G12 (which binds to outer domain glycan), VRC01, PG9, and b12.

In some embodiments, this disclosure provides a method of preventing or treating an HIV infection or an HIV-related disease. The method comprises (a) identifying a patient in need of such prevention or treatment, and (b) administering to the patient a first therapeutic agent comprising a therapeutically effective amount of at least one anti-HIV antibody, or antigen-binding portion thereof, as described above.

In some embodiments, the method further comprises administering a second therapeutic agent. In some embodiments, the second therapeutic agent comprises an antiviral agent or one or more additional antibodies. In some embodiments, the one or more additional antibodies comprise a second anti-HIV antibody (e.g., an isolated anti-HIV bNAb as disclosed) or antigen binding portion thereof, or a third antibody binding to a third antigen. In some embodiments, the third antigen is selected from the group consisting of CD3, FcγRI (CD64), FcγRII (CD32), FcγRIII (CD 16); CD89, CCR5, CD4, CD8, CD28, CD137, CTLA-4, gp41, killer cell immunoglobulin like receptor, three Ig domains and long cytoplasmic tail 1 (KIR3DL1), killer cell immunoglobulin like receptor, three Ig domains and long cytoplasmic tail 1 (KIR3DL1), killer cell immunoglobulin like receptor, two Ig domains and long cytoplasmic tail 1 (KIR2DL1), killer cell immunoglobulin like receptor, two Ig domains and long cytoplasmic tail 2 (KIR2DL2), killer cell immunoglobulin like receptor, two Ig domains and long cytoplasmic tail 3 (KIR2DL3), killer cell lectin like receptor C1 (KLRC1), killer cell lectin like receptor C2 (KLRC2), killer cell lectin like receptor C3 (KLRC3), killer cell lectin like receptor C4 (KLRC4), killer cell lectin like receptor D1 (KLRD1), killer cell lectin like receptor K1 (KLRK1), natural cytotoxicity triggering receptor 3 (NCR3 or NKp30), natural cytotoxicity triggering receptor 2 (NCR2 or NK-p44), natural cytotoxicity triggering receptor 1 (NCR1 or NK-p46), CD226 (DNAM-1), cytotoxic and regulatory T cell molecule (CRTAM or CD355), signaling lymphocytic activation molecule family member 1 (SLAMF1), CD48 (SLAMF2), lymphocyte antigen 9 (LY9 or SLAMF3), CD244 (2B4 or SLAMF4), CD84 (SLAMF5), SLAM family member 6 (SLAMF6 or NTB-A), SLAM family member 7 (SLAMF7 or CRACC), CD27 (TNFRSF7), semaphorin 4D (SEMA4D or CD 100), CD 160 (NK1), an immune checkpoint protein (e.g., PD-1, PD-L1, CTLA-4), and a second epitope of gp120.

In some embodiments, the antiviral agent is selected from the group consisting of a non-nucleoside reverse transcriptase inhibitor, a protease inhibitor, an entry or fusion inhibitor, and an integrase inhibitor.

In some embodiments, the first therapeutic agent or the second therapeutic agent is administered to the patient intratumorally, intravenously, subcutaneously, intraosscously, orally, transdermally, or sublingually. In some embodiments, the first therapeutic agent is administered to the patient before, after, or concurrently with the second therapeutic agent. In some embodiments, the antibody or antigen-binding fragment thereof is administered prophylactically or therapeutically.

Passive immunization has proven to be an effective and safe strategy for the prevention and treatment of viral diseases. (See, for example, Keller et al., Clin. Microbiol. Rev. 13:602-14 (2000); Casadevall, Nat. Biotechnol. 20:114 (2002); Shibata et al., Nat. Med. 5:204-10 (1999); and Igarashi et al., Nat. Med. 5:211-16 (1999), each of which is incorporated herein by reference). Passive immunization using human monoclonal antibodies provides an immediate treatment strategy for emergency prophylaxis and treatment of HIV. Subjects at risk for HIV-related diseases or disorders include patients who have come into contact with an infected person or who have been exposed to HIV in some other way. Administration of a prophylactic agent can occur prior to the manifestation of symptoms characteristic of HIV-related disease or disorder, such that a disease or disorder is prevented or, alternatively, delayed in its progression.

For in vivo treatment of human and non-human patients, the patient is administered or provided a pharmaceutical formulation including an HIV antibody of the invention. When used for in vivo therapy, the antibodies of the invention are administered to the patient in therapeutically effective amounts (i.e., amounts that eliminate or reduce the patient's viral burden). The antibodies are administered to a human patient, in accord with known methods, such as intravenous administration, for example, as a bolus or by continuous infusion over a period of time, by intramuscular, intraperitoneal, intracerobrospinal, subcutaneous, intra-articular, intrasynovial, intrathecal, oral, topical, or inhalation routes. The antibodies can be administered parenterally, when possible, at the target cell site, or intravenously. In some embodiments, the antibody is administered by intravenous or subcutaneous administration. Therapeutic compositions of the invention may be administered to a patient or subject systemically, parenterally, or locally. The above parameters for assessing successful treatment and improvement in the disease are readily measurable by routine procedures familiar to a physician.

For parenteral administration, the antibodies may be formulated in a unit dosage injectable form (solution, suspension, emulsion) in association with a pharmaceutically acceptable, parenteral vehicle. Examples of such vehicles include, but are not limited to, water, saline, Ringer's solution, dextrose solution, and 5% human serum albumin. Nonaqueous vehicles include, but are not limited to, fixed oils and ethyl oleate. Liposomes can be used as carriers. The vehicle may contain minor amounts of additives such as substances that enhance isotonicity and chemical stability, such as, for example, buffers and preservatives. The antibodies can be formulated in such vehicles at concentrations of about 1 mg/ml to 10 mg/ml.

The dose and dosage regimen depends upon a variety of factors readily determined by a physician, such as the nature of the infection, for example, its therapeutic index, the patient, and the patient's history. Generally, a therapeutically effective amount of an antibody is administered to a patient. In some embodiments, the amount of antibody administered is in the range of about 0.1 mg/kg to about 50 mg/kg of patient body weight. Depending on the type and severity of the infection, about 0.1 mg/kg to about 50 mg/kg body weight (for example, about 0.1-15 mg/kg/dose) of antibody is an initial candidate dosage for administration to the patient, whether, for example, by one or more separate administrations, or by continuous infusion. The progress of this therapy is readily monitored by conventional methods and assays and based on criteria known to the physician or other persons of skill in the art. The above parameters for assessing successful treatment and improvement in the disease are readily measurable by routine procedures familiar to a physician.

Other therapeutic regimens may be combined with the administration of the HIV antibody of the present invention. The combined administration includes co-administration, using separate formulations or a single pharmaceutical formulation, and consecutive administration in either order, wherein preferably there is a time period while both (or all) active agents simultaneously exert their biological activities. Such combined therapy can result in a synergistic therapeutic effect. The above parameters for assessing successful treatment and improvement in the disease are readily measurable by routine procedures familiar to a physician.

b. Method of Reducing Viral Replication

Methods for reducing an increase in HIV virus titer, virus replication, virus proliferation or an amount of an HIV viral protein in a subject are further provided. According to another aspect, a method includes administering to the subject an amount of an HIV antibody effective to reduce an increase in HIV titer, virus replication or an amount of an HIV protein of one or more HIV strains or isolates in the subject.

According to another embodiment, the present invention provides a method of reducing viral replication or spread of HIV infection to additional host cells or tissues comprising contacting a mammalian cell with the antibody, or a portion thereof, which binds to an antigenic epitope, e.g., an antigenic epitope on gp120.

c. Combination Therapies
(1) Combination Therapies with Two or More Anti-HIV Antibodies

In some embodiments, this disclosure provides a method for treating or preventing an HIV infection in a human subject having, or at risk of having, the HIV infection. The method comprises administering to the human subject a therapeutically effective amount of an antibody or antigen-binding fragment, as disclosed herein, or a pharmaceutical composition thereof, in combination with a therapeutically effective amount of one or more (e.g., one, two, three, one or two, or one to threc) additional therapeutic agents. In one embodiment, a method for treating an HIV infection in a human subject having or at risk of having the infection is provided, the method comprising administering to the human subject a therapeutically effective amount of an antibody or antibodies disclosed herein, or a pharmaceutically acceptable salt thereof, in combination with a therapeutically effective amount of one or more (e.g., one, two, three, one or two, or one to three) additional therapeutic agents.

### Antibody Combination Therapy

In some embodiments, the disclosed antibody or antigen-binding fragment thereof is co-administered with a second anti-HIV antibody. In some embodiments, the antibody or antigen-binding fragment thereof is co-administered with a second anti-HIV antibody that binds to an epitope or region of gp 120 selected from the group consisting of: (i) third variable loop (V3) and/or high mannose patch comprising a N332 oligomannose glycan; (ii) second variable loop (V2) and/or Env trimer apex; (iii) CD4 binding site (CD4bs); (iv) gp120/gp41 interface; or (v) silent face of gp120 . . . . The foregoing epitopes or regions of gp120 bound by broadly neutralizing antibodies are described, e.g., in McCoy, Retrovirology (2018) 15:70; Sok and Burton, Nat Immunol. 2018 19 (11): 1179-1188; Possas, et al., Expert Opin Ther Pat. 2018 July; 28 (7): 551-560; and Stephenson and Barouch, Curr HIV/AIDS Rep (2016) 13:31-37, which are hereby incorporated herein by reference in their entirety for all purposes.

In some embodiments, the combination therapy entails co-administration of an antibody or antigen-binding fragment thereof and another anti-HIV broadly neutralizing antibody or bNAb (i.e., a neutralizing antibody that neutralizes multiple HIV-1 viral strains). Various bNAbs are known in the art and may be used as a combining therapeutic agent. Additional illustrative bNAbs of use include, those that comprise VH and VL that bind to or compete with an epitope or region of gp120 selected from the group consisting of: (i) third variable loop (V3) and/or high mannose patch comprising a N332 oligomannose glycan; (ii) second variable loop (V2) and/or Env trimer apex; (iii) CD4 binding site (CD4bs); (iv) gp120/gp41 interface; or (v) silent face of gp120 . . . . Illustrative bNAbs for use in anti-HIV antibody combination therapies include those that comprise VH and VL that bind to or compete with GS-9722 (clipovimab), PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03 (all of which bind the V3-glycan region), 2F5, 4E10, M66.6, CAP206-CH12, 10E8, 10E8v4, 10E8-5R-100cF, DH511.11P, 7b2, and LN01 (all of which bind the MPER of gp41); PG9, PG16, CH01-04 (all of which bind VIV2-glycan), 2G12 (which binds to outer domain glycan); b12, F105, VRC01, VRC07, VRC07-523, VRC03, VRC06, VRC06b01 VRC08, VRC0801, NIH45-46, GS-9723, GS-5423, 3BNC117, 3BNC60, VRC-PG04, PGV04; CH103, 44-VRC13.01, INC9, 12A12, N6, N6LS (VRC-HIVMAB091-00-AB), N49-P7, NC-Cow1, IOMA, CH235 and CH235.12, N49P6, N49P7, N49P11, N49P9 and N60P25 (all of which bind to the CD4 binding site).

In some embodiments, the combination therapy includes an antibody that binds to an epitope or region of gp 120 in the third variable loop (V3) and/or high mannose patch comprising a N332 oligomannose glycan and competes with or comprises VH and VL regions from an antibody selected from the group consisting of GS-9722 (clipovimab), PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. Additional broadly neutralizing antibodies that bind to gp120 in the third variable loop (V3) and/or high mannose patch comprising a N332 oligomannose glycan and which can be used in the second antigen binding domain of the herein described multi-specific antigen binding molecules are described, e.g., in WO 2012/030904; WO 2014/063059; WO 2016/149698; WO 2017/106346; WO 2018/075564, WO 2018/125813; WO 2018/237148, WO 2019/226829, WO 2020/023827, WO2020/056145 and Kerwin, et al., J Pharm Sci. 2020 January; 109 (1): 233-246, which are hereby incorporated herein by reference in their entireties for all purposes.

In some embodiments, the combination therapy includes an antibody that binds to an epitope or region of gp120 in the CD4 binding site (CD4bs) and competes with or comprises VH and VL regions from an antibody selected from the group consisting of b12, F105, VRC01, VRC07, VRC07-523, VRC03, VRC06, VRC06b01 VRC08, VRC0801, NIH45-46, 3BNC117, GS-9723, GS-5423, 3BNC60, VRC-PG04, PGV04; CH103, 44-VRC13.01, INC9, 12A12, N6, N49-P7, NC-Cow1, IOMA, CH235 and CH235.12, N49P6, N49P7, N49P11, N49P9 and N60P25. Additional broadly neutralizing antibodies that bind to gp 120 in the CD4 binding site (CD4bs) and which can be used in the second antigen binding domain of the herein described multi-specific antigen binding molecules are described, e.g., in WO 2011/038290; WO 2012/158948; WO 2013/016468; WO 2013/192589; WO 2013/086533; WO 2015/128846; WO 2016/149698; WO 2016/149695; WO 2018/075564; WO 2018/125813; WO 2018/237357, and U.S. Pat. Nos. 9,493,549 and 9,879,068. Additional antibodies that bind to an epitope or region of gp120 in the CD4bs that can be combined or co-administered with the present antibodies are described, e.g., in Schommers, et al., Cell (2020) 180:471-489; Freund, et al., Sci Transl Med (2017) 9: caal2144; Diskin, et al., J Exp Med (2013) 210:1235-49; and Scheid, et al., Science (2011) 333:1633-1637. The foregoing publications are hereby incorporated herein by reference in their entireties for all purposes.

In some embodiments, the combination therapy includes an antibody that binds to an epitope or region of gp 120 in the second variable loop (V2) and/or Env trimer apex and competes with or comprises CDRs and/or VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E, and VRC38.01.

In some embodiments, the combination therapy includes an antibody that binds to an epitope or region of gp120 in the gp120/gp41 interface and competes with or comprises CDRs and/or VH and VL regions from an antibody selected from the group consisting of PGT-151, CAP248-2B, 35022, 8ANC195, ACS202, VRC34 and VRC34.01.

In some embodiments, the combination therapy includes an antibody that binds to an epitope or region of the gp120 silent face and competes with or comprises second VH and VL regions from antibody VRC-PG05.

In some embodiments, the combination therapy includes an antibody that binds to an epitope or region of gp41 in the membrane-proximal region (MPER) and competes with or comprises second VH and VL regions from an antibody selected from the group consisting of 10E8, 10E8v4, 10E8-5R-100cF, 4E10, DH511.11P, 2F5, 7b2, and LN01. In some embodiments, the combination therapy includes an antibody that binds to an epitope or region of KLIC (“KLIC”), an immutable site of the transmembrane protein gp41 and competes with or comprises second VH and VL regions from Clone 3 human monoclonal antibody (C13hmAb) (Protheragen). See, e.g., Vanini, et al., AIDS. (1993) 7 (2): 167-74.

In some embodiments, the combination therapy includes an antibody that binds to an epitope or region of the gp41 fusion peptide and competes with or comprises second VH and VL regions from an antibody selected from the group consisting of VRC34 and ACS202.

In some embodiments, the combination therapy includes a multi-specific, e.g., a bispecific or tri-specific antibody that binds to an HIV antigen. Examples of HIV bispecific and trispecific antibodies include MGD014, B12BiTe, BiIA-SG, TMB-bispecific, SAR-441236, VRC-01/PGDM-1400/10E8v4, 10E8.4/iMab, and 10E8v4/PGT121-VRC01.

Prior to administration, the bNAbs may be improved to have enhanced drug-like-properties, reduced immunogenicity, enhanced ADCC, and suitable pharmacokinetic properties. Such antibodies were shown to bind to the HIV envelope glycoprotein expressed on the surface of virion or infected cells and mediate both direct neutralization of the virus as well as potent NK, Monocyte and PBMC killing of these cells. This property allows the antibodies to treat HIV infections by neutralizing the virus, and also kill and eliminate latently HIV infected cells in infected individuals, potentially leading to a sterilizing cure for HIV.

In various embodiments, all antibodies administered in a combination anti-HIV antibody therapy can have Fc and/or post-translational modifications that increase serum half-life and/or enhance effector activity, as described above.

In various embodiments, the antibody or antigen-binding fragments, and optionally combined bNAbs, can be in vivo delivered, e.g., expressed in vivo from administered mRNA or engineered B-cells. Examples of in vivo delivered bNAbs include AAV8-VRC07; mRNA encoding anti-HIV antibody VRC01; and engineered B-cells encoding 3BNC117 (Hartweger et al., J. Exp. Med. 2019, 1301).

(2) Combination Therapies with Other Anti-HIV Therapeutic Agents

In some embodiments, a method for treating or preventing an HIV infection in a human having or at risk of having the infection is provided, comprising administering to the human a therapeutically effective amount of the antibody or antigen-binding fragments, as disclosed herein, in combination with a therapeutically effective amount of one or more (e.g., one, two, three, one or two, or one to three) additional therapeutic agents. In one embodiment, a method for treating an HIV infection in a human having or at risk of having the infection is provided, comprising administering to the human a therapeutically effective amount of the antibody or antigen-binding fragments, as disclosed herein, in combination with a therapeutically effective amount of one or more (e.g., one, two, three, one or two, or one to three) additional therapeutic agents.

In one embodiment, pharmaceutical compositions comprising the antibody or antigen-binding fragments, as disclosed herein, in combination with one or more (e.g., one, two, three, one or two, or one to three) additional therapeutic agents and a pharmaceutically acceptable carrier, diluent, or excipient are provided.

In some embodiments, provided are methods for treating an HIV infection, comprising administering to a patient in need thereof a therapeutically effective amount of the antibody or antigen-binding fragment thereof, as described herein, in combination with a therapeutically effective amount of one or more additional therapeutic agents which are suitable for treating an HIV infection.

In some embodiments, the antibody or antigen-binding fragment thereof is combined with one, two, three, four, or more additional therapeutic agents. In some embodiments, the antibody or antigen-binding fragment thereof is combined with two additional therapeutic agents. In other embodiments, the antibody or antigen-binding fragment thereof is combined with three additional therapeutic agents. In further embodiments, the antibody or antigen-binding fragment thereof is combined with four additional therapeutic agents. The one, two, three, four, or more additional therapeutic agents can be different therapeutic agents selected from the same class of therapeutic agents (e.g., one or more anti-HIV broadly neutralizing antibodies) and/or they can be selected from different classes of therapeutic agents.

### Administration of HIV Combination Therapy

In some embodiments, the antibody or antigen-binding fragment thereof, as described herein, is co-administered with one or more additional therapeutic agents. Co-administration of an antibody or antigen-binding fragment disclosed herein with one or more additional therapeutic agents generally refers to simultaneous or sequential administration of the antibodies or antigen-binding fragments disclosed herein and one or more additional therapeutic agents, such that therapeutically effective amounts of the antibodies or antigen-binding fragments disclosed herein and the one or more additional therapeutic agents are both present in the body of the patient. When administered sequentially, the combination may be administered in two or more administrations.

Co-administration includes concurrent administration as well as administration of unit dosages of the antibody or antigen-binding fragment thereof, as described herein, before or after administration of unit dosages of one or more additional therapeutic agents. For example, the antibody or antigen-binding fragment thereof, as described herein, may be administered within seconds, minutes, hours or days of the administration of the one or more additional therapeutic agents. In some embodiments, a unit dose of an antibody or antigen-binding fragment disclosed herein is administered first, followed within seconds, minutes, hours or days by administration of a unit dose of one or more additional therapeutic agents. Alternatively, a unit dose of one or more additional therapeutic agents is administered first, followed by administration of a unit dose of the antibodies or antigen-binding fragments disclosed herein within seconds, minutes, hours or days. In other embodiments, a unit dose of an antibody or antigen-binding fragment disclosed herein is administered first, followed, after a period of hours (e.g., 1-12 hours, 1-24 hours, 1-36 hours, 1-48 hours, 1-60 hours, 1-72 hours), by administration of a unit dose of one or more additional therapeutic agents. In yet other embodiments, a unit dose of one or more additional therapeutic agents is administered first, followed, after a period of hours (e.g., 1-12 hours, 1-24 hours, 1-36 hours, 1-48 hours, 1-60 hours, 1-72 hours), by administration of a unit dose of an antibody or antigen-binding fragment disclosed herein.

In some embodiments, an antibody or antigen-binding fragment disclosed herein is combined with one or more additional therapeutic agents in a unitary dosage form for simultaneous administration to a patient, for example, as a solid, liquid or suspension dosage form for oral, intravenous, intramuscular or subcutaneous administration.

In some embodiments, the antibodies or antigen-binding fragments are formulated as a liquid solution or suspension, which may optionally contain one or more other compounds useful for treating HIV. In some embodiments, the liquid solution or suspension can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In some embodiments, such liquid solutions or suspensions are suitable for once daily, once weekly (i.e., QW), once bi-weekly (i.e., once every other week, or once every two weeks or Q2W), once monthly (i.e., QM) or once bi-monthly dosing (i.e., once every other month, or once every two months or Q2M) dosing or administration intervals. In some embodiments, the antibodies or antigen-binding fragments are administered once daily, once weekly (i.e., QW), once bi-weekly (i.e., once every other week, or once every two weeks or Q2W), once monthly (i.e., QM), once bi-monthly dosing (i.e., once every other month, or once every two months or Q2M), once every three months (i.e., Q3M), once every four months (i.e., Q4M),

### HIV Combination Therapy

In the above embodiments, the additional therapeutic agent may be an anti-HIV agent. HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, HIV Tat or Rev inhibitors, immunomodulators, (e.g., immunostimulators), immunotherapeutic agents, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies), latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, TAT protein inhibitors, HIV-1 Nef modulators (e.g., Nef inhibitors), Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-4 inhibitors, CDK-6 inhibitors, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, TAT protein inhibitors, prolylendopeptidase inhibitors, Phospholipase A2 inhibitors, pharmacokinetic enhancers, HIV gene therapy, TNF alpha ligand inhibitors, IFN antagonists, HIV vaccines, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, HIV capsid inhibitors, HIV Tat or Rev inhibitors, immunomodulators, (e.g., immunostimulators), immunotherapeutic agents, immune-based therapies, PI3K inhibitors, HIV antibodies, and bispecific antibodies, and “antibody-like” therapeutic proteins, and combinations thereof.

### HIV Combination Drugs

In some embodiments, the antibodies or antigen-binding fragments described herein are combined with one, two, three, four or more additional anti-HIV therapeutic agents. Example anti-HIV therapeutic agents that can be combined include without limitation ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY (bictegravir+emtricitabine+tenofovir alafenamide), adefovir; adefovir dipivoxil; cobicistat; emtricitabine; tenofovir; tenofovir alafenamide and elvitegravir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXAR; abacavir sulfate and lamivudine; ABC+3TC); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine; cabotegravir+rilpivirine; elpida (elsulfavirine; VM-1500; VM-1500A); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; efavirenz, lamivudine, and emtricitabine; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

### Other HIV Drugs

Examples of other drugs for treating HIV that can be combined with an agent of this disclosure include aspernigrin C, acemannan, alisporivir, BanLec, deferiprone, Gamimune, metenkefalin, naltrexone, Prolastin, REP 9, RPI-MN, VSSP, Hlviral, SB-728-T, 1,5-dicaffeoylquinic acid, rHIV7-shl-TAR-CCR5RZ, AAV-cCD4-Ig gene therapy, MazF gene therapy, BlockAide, bevirimat derivatives, ABX-464, AG-1105, APH-0812, bryostatin analogs, BIT-225, CYT-107, CS-TATI-1, fluoro-beta-D-arabinose nucleic acid (FANA)-modified antisense oligonucleotides, FX-101, griffithsin, HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591 (islatravir), NOV-205, OB-002H, ODE-Bn-TFV, MI-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, x1-081, rfhSP-D, [18F]-MC-225, URMC-099-C, RES-529, and VIR-576.

### HIV Protease Inhibitors

In some embodiments, the antibodies or antigen-binding fragments described herein are combined with an HIV protease inhibitor. Examples of HIV protease inhibitors include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, AEBL-2, DG-17, GS-1156, TMB-657 (PPL-100), T-169, BL-008, MK-8122, TMB-607, GRL-02031, and TMC-310911.

### HIV Ribonuclease H Inhibitors

In some embodiments, the antibodies or antigen-binding fragments described herein are combined with an HIV ribonuclease H inhibitor. Examples of HIV ribonuclease H inhibitors that can be combined include NSC-727447.

### HIV Nef Inhibitors

In some embodiments, the antibodies or antigen-binding fragments described herein are combined with an HIV Nef inhibitor. Examples of HIV Nef inhibitors that can be combined with include FP-1.

### HIV Reverse Transcriptase Inhibitors

In some embodiments, the antibodies or antigen-binding fragments described herein are combined with a non-nucleoside or non-nucleotide inhibitor. Examples of HIV non-nucleoside or non-nucleotide inhibitors of reverse transcriptase include dapivirine, delavirdine, delavirdine mesylate, doravirine, efavirenz, ctravirine, lentinan, nevirapine, rilpivirine, ACC-007, ACC-008, AIC-292, F-18, KM-023, PC-1005, VM-1500A-LAI, PF-3450074, elsulfavirine (sustained release oral, HIV infection), elsulfavirine (long-acting injectable nanosuspension, HIV infection), and elsulfavirine (VM-1500).

In some embodiments, the antibodies or antigen-binding fragments described herein are combined with an HIV nucleoside or nucleotide inhibitor. Examples of HIV nucleoside or nucleotide inhibitors of reverse transcriptase include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, ctravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8591, MK-858, VM-2500, and KP-1461.

### HIV Integrase Inhibitors

In some embodiments, the antibodies or antigen-binding fragments described herein are combined with an HIV integrase inhibitor. Examples of HIV integrase inhibitors include elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long-acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T-169, STP-0404, VM-3500, and cabotegravir.

In some embodiments, the antibodies or antigen-binding fragments described herein are combined with an HIV non-catalytic site, or allosteric, integrase inhibitor (NCINI). Examples of HIV non-catalytic site, or allosteric, integrase inhibitors (NCINI) include CX-05045, CX-05168, and CX-14442.

### HIV Entry Inhibitors

In some embodiments, the antibodies or antigen-binding fragments described herein are combined with an HIV entry inhibitor. Examples of HIV entry (fusion) inhibitors include AAR-501, LBT-5001, cenicriviroc, CCR5 inhibitors, gp41 inhibitors, CD4 attachment inhibitors, gp120 inhibitors, gp160 inhibitors, and CXCR4 inhibitors.

In some embodiments, the antibodies or antigen-binding fragments described herein are combined with a CCR5 inhibitor. Examples of CCR5 inhibitors include aplaviroc, vicriviroc, maraviroc, maraviroc (long-acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc, and vMIP (Haimipu).

In some embodiments, the antibodies or antigen-binding fragments described herein are combined with a CXCR4 inhibitor. Examples of CXCR4 inhibitors include plerixafor, ALT-1188, N15 peptide, and vMIP (Haimipu).

In some embodiments, the antibodies or antigen-binding fragments described herein are combined with a gp41 inhibitor. Examples of gp41 inhibitors include albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, PIE-12 trimer, and sifuvirtide.

In some embodiments, the antibodies or antigen-binding fragments described herein are combined with a CD4 attachment inhibitor. Examples of CD4 attachment inhibitors include ibalizumab and CADA analogs

In some embodiments, the antibodies or antigen-binding fragments described herein are combined with a gp120 inhibitor. Examples of gp120 inhibitors include anti-HIV microbicide, Radha-108 (receptol) 3B3-PE38, BanLec, bentonite-based nanomedicine, fostemsavir tromethamine, IQP-0831, VVX-004, and BMS-663068.

In some embodiments, the antibodies or antigen-binding fragments described herein are combined with a gp160 inhibitor. Examples of gp 160 inhibitors that can be combined include fangchinoline.

### HIV Maturation Inhibitors

In some embodiments, the antibodies or antigen-binding fragments described herein are combined with an HIV maturation inhibitor. Examples of HIV maturation inhibitors include BMS-955176, GSK-3640254, and GSK-2838232.

### Latency Reversing Agents

In some embodiments, the antibodies or antigen-binding fragments described herein are combined with an HIV latency reversing agent. Examples of latency reversing agents that can be combined with the one or more multi-specific antigen binding molecules, described herein, include IL-15 receptor agonists (e.g., ALT-803; interleukin-15/Fc fusion protein (e.g., XmAb24306); recombinant interleukin-15 (e.g., AM0015, NIZ-985); pegylated IL-15 (e.g., NKTR-255)); toll-like receptor (TLR) agonists (including TLR7 agonists, e.g., GS-9620 and TLR8 agonists, e.g., GS-9688), histone deacetylase (HDAC) inhibitors, proteasome inhibitors such as velcade, protein kinase C (PKC) activators, Smyd2 inhibitors, BET-bromodomain 4 (BRD4) inhibitors, ionomycin, IAP antagonists (inhibitor of apoptosis proteins, such as APG-1387, LBW-242), SMAC mimetics (including ciapavir, TL32711, LCL161, GDC-0917, HGS1029, AT-406), Debio-1143, PMA, SAHA (suberanilohydroxamic acid, or suberoyl, anilide, and hydroxamic acid), NIZ-985, IL-15 modulating antibodies, (including IL-15, IL-15 fusion proteins and IL-15 receptor agonists, e.g., ALT-803), JQ1, disulfiram, amphotericin B, and ubiquitin inhibitors such as largazole analogs, APH-0812, and GSK-343. Examples of HDAC inhibitors include romidepsin, vorinostat, and panobinostat. Examples of PKC activators include indolactam, prostratin, ingenol B, and DAG-lactones.

### Toll-Like Receptor (TLR) Agonists

In some embodiments, the antibodies or antigen-binding fragments described herein are combined with an agonist of a toll-like receptor (TLR), e.g., an agonist of TLRI (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR 10 (NCBI Gene ID: 81793). Example TLR7 agonists that can be co-administered or combined with the one or more multi-specific antigen binding molecules, described herein, include without limitation AL-034, DSP-0509, GS-9620 (vesatolimod), vesatolimod analogs, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). A TLR7/TLR8 agonist that can be co-administered is NKTR-262, telratolimod and BDB-001. Example TLR8 agonists that can be co-administered or combined with the one or more multi-specific antigen binding molecules, described herein, include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. Examples of TLR4 agonist include G-100, and GSK-1795091.

### Histone Deacetylase (HDAC) Inhibitors

In some embodiments, the antibodies or antigen-binding fragments described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Cyclin-Dependent Kinase (CDK) Inhibitors or Antagonists

In some embodiments, the antibodies or antigen-binding fragments described herein are combined with an inhibitor or antagonist of a cyclin-dependent kinase (CDK), e.g., cyclin-dependent kinase 4 (CDK4; NCBI Gene ID: 1019), cyclin-dependent kinase 6 (CDK6; NCBI Gene ID: 1021), cyclin-dependent kinase 9 (CDK9; NCBI Gene ID: 1025). In some embodiments, the CDK4/CDK6/CDK9 inhibitor or antagonist is selected from the group consisting of VS2-370.

### Stimulator of Interferon Genes (STING) Agonists

In some embodiments, the antibodies or antigen-binding fragments described herein are combined with a stimulator of interferon gene (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP), and cyclic-di-AMP.

### RIG-I Agonists

In some embodiments, the antibodies or antigen-binding fragments described herein are combined with an agonist of DExD/H-box helicase 58 (DDX58; a.k.a., RIG-I, RIG1, RIG1, RLR-1, SGMRT2; NCBI Gene ID: 23586). In some embodiments, the agents described herein are combined with a RIG-I modulator such as RGT-100, or NOD2 modulator, such as SB-9200 (a.k.a., GS 9992; inarigivir), and IR-103. An illustrative RIG-I agonist is KIN1148, described by Hemann, et al., J Immunol May 1, 2016, 196 (1 Supplement) 76.1. Additional RIG-I agonists are described, e.g., in Elion, et al., Cancer Res. (2018) 78 (21): 6183-6195; and Liu, et al., J Virol. (2016) 90 (20): 9406-19. RIG-I agonists are commercially available, e.g., from Invivogen (invivogen.com).

### LAG-3 and TIM-3 Inhibitors

In some embodiments, the antibodies or antigen-binding fragments described herein are combined with an anti-TIM-3 (a.k.a., hepatitis A virus cellular receptor 2 antibody (HAVCR2; NCBI Gene ID: 84868), such as TSR-022, LY-3321367, MBG-453, INCAGN-2390. In some embodiments, the antibodies or antigen-binding fragments described herein are combined with an anti-LAG-3 (Lymphocyte-activation) (NCBI Gene ID: 3902) antibody, such as relatlimab (ONO-4482), LAG-525, MK-4280, REGN-3767, INCAGN2385.

### Capsid Inhibitors

In some embodiments, the antibodies or antigen-binding fragments described herein are combined with a capsid inhibitor. Examples of capsid inhibitors that can be combined with an agent of this disclosure include capsid polymerization inhibitors or capsid disrupting compounds, HIV nucleocapsid p7 (NCp7) inhibitors such as azodicarbonamide, HIV p24 capsid protein inhibitors, GS-6207 (lenacapavir), GS-CA1, AVI-621, AVI-101, AVI-201, AVI-301, and AVI-CAN1-15 series, PF-3450074, and compounds described in Intl. Patent Publ. No. WO 2019/087016.

### Immune-Based Therapies

In some embodiments, the antibodies or antigen-binding fragments described herein are combined with an immune-based therapy. Examples of immune-based therapies include toll-like receptor (TLR) modulators such as TLR1, TLR2, TLR3, TLR4, TLR5, TLR6, TLR7, TLR8, TLR9, TLR10, TLR11, TLR12, AND TLR13; programmed cell death protein 1 (PD-1) modulators; programmed death-ligand 1 (PD-L1) modulators; IL-15 modulators (e.g., IL-15 receptor agonists (e.g., ALT-803; interleukin-15/Fc fusion protein (e.g., XmAb24306); recombinant interleukin-15 (e.g., AM0015, NIZ-985); pegylated IL-15 (e.g., NKTR-255)); DermaVir; interleukin-7; plaquenil (hydroxychloroquine); prolcukin (aldesleukin, IL-2); interferon alfa; interferon alfa-2b; interferon alfa-n3; pegylated interferon alfa; interferon gamma; hydroxyurca; mycophenolate mofetil (MPA) and its ester derivative mycophenolate mofetil (MMF); ribavirin; polymer polyethylencimine (PEI); gepon; IL-12; WF-10; VGV-1; MOR-22; BMS-936559; CYT-107, normferon, peginterferon alfa-2a, peginterferon alfa-2b, RPI-MN, STING modulators, RIG-I modulators, NOD2 modulators, SB-9200, and IR-103.

In some embodiments, the antibodies or antigen-binding fragments described herein are combined with a TLR agonist. Examples of TLR agonists include without limitation: vesatolimod (GS-9620), lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod.

### CD47 Targeting Agents

In various embodiments, the antibodies or antigen-binding fragments described herein are combined with an agent that disrupts the binding of CD47 to SIRPα, e.g., an agent that targets CD47 or an agent that targets SIRPα. In various embodiments, the antibodies or antigen-binding fragments described herein are combined with an inhibitor of CD47 (IAP, MER6, OA3; NCBI Gene ID: 961). Examples of CD47 inhibitors include without limitation anti-CD47 mAbs (Vx-1004), anti-human CD47 mAbs (CNTO-7108), CC-90002, CC-90002-ST-001, humanized anti-CD47 antibody (Hu5F9-G4; magrolimab), NI-1701, NI-1801, RCT-1938, and TTI-621. In some embodiments, the CD47 inhibitor is magrolimab.

### Immune Checkpoint Receptor Protein Modulators

In various embodiments, the antibodies or antigen-binding fragments described herein are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune checkpoints can augment the effect of immune checkpoint inhibitors in infective therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18 (11): 671-688).

Examples of immune checkpoint proteins or receptors that can be combined with the antibodies or antigen-binding fragments described herein include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-sct domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript IE (RAETIE; ULBP4); retinoic acid early transcript 1G (RAETIG; ULBP5); retinoic acid early transcript IL (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and SLAM family member 7 (SLAMF7).

In various embodiments, the antibodies or antigen-binding fragments described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCDILG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the antibodies or antigen-binding fragments described herein are combined with one or more agonists or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). Scc, e.g., Xu, et al., J Exp Clin Cancer Res. (2018) 37:110.

In various embodiments, the antibodies or antigen-binding fragments described herein are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin-like receptor D1 (KLRD1, CD94). In various embodiments, the antibodies or antigen-binding fragments described herein are combined with one or more agonists or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). Sec, e.g., Davis, et al., Semin Immunol. (2017) 31:64-75; Fang, et al., Semin Immunol. (2017) 31:37-54; and Chiossone, et al., Nat Rev Immunol. (2018) 18 (11): 671-688.

[In some embodiments, the one or more immune checkpoint inhibitors comprises a proteinaccous (e.g., antibody or fragment thereof, or antibody mimctic) inhibitor of PD-L1 (CD274), PD-1 (PDCD1) or CTLA4. In some embodiments, the one or more immune checkpoint inhibitors comprise a small organic molecule inhibitor of PD-L1 (CD274), PD-1 (PDCD1) or CTLA4.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFB-EC domain), CA-170 (PD-L1/VISTA), CDX-527 (CD27/PD-L1), LY-3415244 (TIM3/PDL1), and INBRX-105 (4-1BB/PDL1).

In some embodiments, the small molecule inhibitor of CD274 or PDCD1 is selected from the group consisting of GS-4224, GS-4416, INCB086550, and MAX10181. In some embodiments, the small molecule inhibitor of CTLA4 comprises BPI-002.

In various embodiments, the antibodies or antigen-binding fragments as described herein are combined with anti-TIGIT antibodies, such as etigilimab, BMS-986207, tiragolumab (a.k.a., MTIG-7192A; RG-6058; RO 7092284), AGEN1307, AGEN1327, AGEN1777, COM-902, IBI-939, AB154, MG1131 and EOS884448 (EOS-448).

### TNF Receptor Superfamily (TNFRSF) Member Agonists or Activators

In various embodiments, the antibodies or antigen-binding fragments described herein are combined with an agonist of one or more TNF receptor superfamily (TNFRSF) members, e.g., an agonist of one or more of TNFRSFIA (NCBI Gene ID: 7132), TNFRSFIB (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Genc ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Genc ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Genc ID: 8718).

Example anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include without limitation RG7876, SEA-CD40, APX-005M, and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include without limitation urelumab, utomilumab (PF-05082566), AGEN2373, and ADG-106.

Example anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) are co-administered. Such antibodies are described, e.g., in WO2017096179 and WO2018089628.

### Interleukin Receptor Agonists

In some embodiments, the antibodies or antigen-binding fragments described herein are combined with an interleukin receptor agonist, such as IL-2, IL-7, IL-15, IL-10, IL-12 agonists; examples of IL-2 receptor agonists such as proleukin (aldesleukin, IL-2); pegylated IL-2 (e.g., NKTR-214); modified variants of IL-2 (e.g., THOR-707), bempegaldesleukin, AIC-284, ALKS-4230, CUI-101, Neo-2/15; IL-15 receptor agonists, such as ALT-803, NKTR-255, and hetIL-15, interleukin-15/Fc fusion protein, AM-0015, NIZ-985, SO-C101, IL-15 Synthorin (pegylated IL-15), P-22339, and a IL-15-PD-1 fusion protein N-809; examples of IL-7 include CYT-107.

Examples of additional interleukin receptor agonists that can be combined with the antibodies or antigen-binding fragments described herein include interferon alfa; interferon alfa-2b; interferon alfa-n3; pegylated interferon alfa; interferon gamma; Flt3 agonists such as CDX-301; gepon; normferon, peginterferon alfa-2a, peginterferon alfa-2b, RPI-MN.

### Bi- and Tri-Specific Natural Killer (NK)-Cell Engagers

In various embodiments, the antibodies or antigen-binding fragments described herein are combined with a bi-specific NK-cell engager (BiKE) or a tri-specific NK-cell engager (TrikE) (e.g., not having an Fc) or bi-specific antibody (e.g., having an Fc) against an NK cell activating receptor, e.g., CD16A, C-type lectin receptors (CD94/NKG2C, NKG2D, NKG2E/H, and NKG2F), natural cytotoxicity receptors (NKp30, NKp44, and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (4-1BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. BiKEs and TriKEs are described, e.g., in Felices, et al., Methods Mol Biol. (2016) 1441:333-346; Fang, et al., Semin Immunol. (2017) 31:37-54.

Examples of a trispecific NK cell engager (TRIKE) include OXS-3550, and CD16-IL-15-B7H3 TriKe.

### Phosphatidylinositol 3-Kinase (PI3K) Inhibitors

In some embodiments, the antibodies or antigen-binding fragments described herein are combined with a PI3K inhibitor. Examples of PI3K inhibitors include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

alpha-4/beta-7 antagonists

In some embodiments, the antibodies or antigen-binding fragments described herein are combined with an alpha-4/beta-7 antagonist. Examples of Integrin alpha-4/beta-7 antagonists include PTG-100, TRK-170, abrilumab, ctrolizumab, carotegrast methyl, and vedolizumab.

### Pharmacokinetic Enhancers

In some embodiments, the antibodies or antigen-binding fragments described herein are combined with a pharmacokinetic enhancer. Examples of pharmacokinetic enhancers include cobicistat and ritonavir.

### Additional Therapeutic Agents

Examples of additional therapeutic agents include the compounds disclosed in WO 2004/096286 (Gilead Sciences); WO 2006/015261 (Gilead Sciences); WO 2006/110157 (Gilead Sciences); WO 2012/003497 (Gilead Sciences); WO 2012/003498 (Gilead Sciences); WO 2012/145728 (Gilead Sciences); WO 2013/006738 (Gilead Sciences); WO 2013/159064 (Gilead Sciences); WO 2014/100323 (Gilead Sciences), US 2013/0165489 (University of Pennsylvania), US 2014/0221378 (Japan Tobacco), US 2014/0221380 (Japan Tobacco); WO 2009/062285 (Boehringer Ingelheim); WO 2010/130034 (Bochringer Ingelheim); WO 2013/006792 (Pharma Resources), US20140221356 (Gilead Sciences), US20100143301 (Gilead Sciences) and WO 2013/091096 (Bochringer Ingelheim).

### HIV Combination Therapy

In a particular embodiment, the antibodies or antigen-binding fragments described herein are combined with one, two, three, four or more additional therapeutic agents selected from ATRIPLA® (cfavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXAR; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; ctravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

It will be appreciated by one of skill in the art that the additional therapeutic agents listed above may be included in more than one of the classes listed above. The particular classes are not intended to limit the functionality of those compounds listed in those classes.

In a specific embodiment, the antibodies or antigen-binding fragments described herein are combined with an HIV nucleoside or nucleotide inhibitor of reverse transcriptase and an HIV non-nucleoside inhibitor of reverse transcriptase. In another specific embodiment, the antibodies or antigen-binding fragments described herein are combined with an HIV nucleoside or nucleotide inhibitor of reverse transcriptase, and an HIV protease inhibiting compound. In an additional embodiment, the antibodies or antigen-binding fragments described herein are combined with an HIV nucleoside or nucleotide inhibitor of reverse transcriptase, an HIV non-nucleoside inhibitor of reverse transcriptase, and a pharmacokinetic enhancer. In some embodiments, the antibodies or antigen-binding fragments described herein are combined with at least one HIV nucleoside inhibitor of reverse transcriptase, an integrase inhibitor, and a pharmacokinetic enhancer. In another embodiment, the antibodies or antigen-binding fragments described herein are combined with two HIV nucleoside or nucleotide inhibitors of reverse transcriptase.

In a particular embodiment, the antibodies or antigen-binding fragments described herein are combined with abacavir sulfate, tenofovir, tenofovir disoproxil, tenofovir disoproxil fumarate, tenofovir disoproxil hemifumarate, tenofovir alafenamide, or tenofovir alafenamide hemifumarate.

In a particular embodiment, the antibodies or antigen-binding fragments described herein are combined with tenofovir, tenofovir disoproxil, tenofovir disoproxil fumarate, tenofovir alafenamide, or tenofovir alafenamide hemifumarate.

In a particular embodiment, the antibodies or antigen-binding fragments described herein are combined with a first additional therapeutic agent selected from the group consisting of abacavir sulfate, tenofovir, tenofovir disoproxil, tenofovir disoproxil fumarate, tenofovir alafenamide, and tenofovir alafenamide hemifumarate, and a second additional therapeutic agent selected from the group consisting of emtricitabine and lamivudine.

In a particular embodiment, the antibodies or antigen-binding fragments described herein are combined with a first additional therapeutic agent selected from the group consisting of tenofovir, tenofovir disoproxil, tenofovir disoproxil fumarate, tenofovir alafenamide, and tenofovir alafenamide hemifumarate, and a second additional therapeutic agent, wherein the second additional therapeutic agent is emtricitabine.

In some embodiments, the antibodies or antigen-binding fragments described herein are combined with one or more additional therapeutic agents in a therapeutically effective dosage amount in the range of, e.g., from 1 mg to 50 mg, 75 mg, 100 mg, 150 mg, 200 mg, 250 mg, 300 mg, 400 mg, 500 mg, 1000 mg or 1500 mg of the antibody or antigen-binding fragment. In some embodiments, the antibodies or antigen-binding fragments described herein are combined with one or more additional therapeutic agents in a therapeutically effective dosage amount in the range of e.g., from about 0.1 mg/kg to about 0.5 mg/kg, 1 mg/kg, 2 mg/kg, 3 mg/kg, 4 mg/kg, 5 mg/kg, 8 mg/kg, 10 mg/kg, 15 mg/kg, 20 mg/kg, 25 mg/kg, 30 mg/kg, 35 mg/kg, 40 mg/kg, 45 mg/kg or 50 mg/kg of the antibody or antigen-binding fragment. In some embodiments, the antibodies or antigen-binding fragments described herein are combined with one or more additional therapeutic agents in a therapeutically effective dosage amount in the range of, e.g., from about 5 mg to about mg, 20 mg, 25 mg, 50 mg, 100 mg, 125 mg, 150 mg, 250 mg, 300 mg, 500 mg, 1000 mg or 1500 mg of the antibody or antigen-binding fragment.

In some embodiments, the antibodies or antigen-binding fragments described herein are combined with 5-30 mg tenofovir alafenamide fumarate, tenofovir alafenamide hemifumarate, or tenofovir alafenamide, and 200 mg emtricitabine. In some embodiments, the antibodies or antigen-binding fragments described herein are combined with 5-10, 5-15, 5-20, 5-25, 25-30, 20-30, 15-30, or 10-30 mg tenofovir alafenamide fumarate, tenofovir alafenamide hemifumarate, or tenofovir alafenamide, and 200 mg emtricitabine. In some embodiments, the antibodies or antigen-binding fragments described herein are combined with 10 mg tenofovir alafenamide fumarate, tenofovir alafenamide hemifumarate, or tenofovir alafenamide, and 200 mg emtricitabine. In some embodiments, the antibodies or antigen-binding fragments described herein are combined with 25 mg tenofovir alafenamide fumarate, tenofovir alafenamide hemifumarate, or tenofovir alafenamide, and 200 mg emtricitabine. In some embodiments, the antibodies or antigen-binding fragments described herein are combined with the agents provided herein in any dosage amount of the antibodies or antigen-binding fragments (e.g., from 1 mg to 500 mg of the antibodies or antigen-binding fragments, as described herein) the same as if each combination of dosages were specifically and individually listed.

In some embodiments, the antibodies or antigen-binding fragments described herein are combined with 200-400 mg tenofovir disoproxil fumarate, tenofovir disoproxil hemifumarate, or tenofovir disoproxil, and 200 mg emtricitabine. In some embodiments, the antibodies or antigen-binding fragments described herein are combined with 200-250, 200-300, 200-350, 250-350, 250-400, 350-400, 300-400, or 250-400 mg tenofovir disoproxil fumarate, tenofovir disoproxil hemifumarate, or tenofovir disoproxil, and 200 mg emtricitabine. In some embodiments, the antibodies or antigen-binding fragments described herein are combined with 300 mg tenofovir disoproxil fumarate, tenofovir disoproxil hemifumarate, or tenofovir disoproxil, and 200 mg emtricitabine. The antibodies or antigen-binding fragments may be combined with the agents provided herein in any dosage amount (e.g., from 1 mg to 500 mg of the antibodies or antigen-binding fragments) the same as if each combination of dosages were specifically and individually listed.

### Long-Acting HIV Inhibitors

In some embodiments, the antibodies or antigen-binding fragments described herein can be co-administered with a long-acting HIV inhibitor. Examples of drugs that are being developed as long acting HIV inhibitors include without limitation: cabotegravir LA, rilpivirine LA, any integrase LA, VM-1500 LAI, maraviroc (LAI), tenofovir implant, MK-8591 implant, long-acting dolutegravir.

In one embodiment, kits comprise the antibodies or antigen-binding fragments described herein in combination with one or more (e.g., one, two, three, one or two, or one to three) additional therapeutic agents.

### HIV Vaccines

In some embodiments, the antibodies or antigen-binding fragments described herein are combined with an HIV vaccine. Examples of HIV vaccines include peptide vaccines, recombinant subunit protein vaccines, live vector vaccines, DNA vaccines, HIV MAG DNA vaccines, CD4-derived peptide vaccines, vaccine combinations, adenoviral vector vaccines (e.g., Ad5, Ad26 or Ad35), simian adenovirus (chimpanzee, gorilla, rhesus i.e., rhAd), adeno-associated virus vector vaccines, chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, GoriIIa adenovirus vaccines, lentiviral vector based vaccine, bi-segmented or tri-segmented arenavirus based vaccines (e.g., LCMV, Pichinde), trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as Vesicular stomatitis virus (VSV) and marabavirus; recombinant human CMV (rhCMV) based vaccine, alphavirus-based vaccines, such as semliki forest virus, venezuelan equine encephalitis virus and sindbis virus (see, e.g., Lauer, et al., Clin Vaccine Immunol. (2017) 24 (1): c00298-16); LNP formulated mRNA based therapeutic vaccines; and LNP-formulated self-replicating RNA/self-amplifying RNA vaccines.

Examples of HIV vaccines include without limitation anti-CD40.Env-gp140 vaccine, Ad4-EnvC150, BG505 SOSIP.664 gp140 adjuvanted vaccine, BG505 SOSIP.GT1.1 gp140 adjuvanted vaccine, Chimigen HIV vaccine, ConM SOSIP.v7 gp140, rgp120 (AIDSVAX), ALVAC HIV (vCP1521)/AIDSVAX B/E (gp120) (RV144), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmunc, GTU-multiHIV (FIT-06), ChAdV63.HIVconsv, gp140 [delta] V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PTI, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines, gag-based DNA vaccine, GI-2010, gp41 HIV-1 vaccine, HIV vaccine (PIKA adjuvant), I i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, recombinant peptide vaccine (HIV infection), NCI, rgp 160 HIV vaccine, RNActive HIV vaccine, SCB-703, Tat Oyi vaccine, TBC-M4, therapeutic HIV vaccine, UBI HIV gp120, Vacc-4x+romidepsin, variant gp 120 polypeptide vaccine, rAd5 gag-pol env A/B/C vaccine,. DNA.HTI and MVA.HTI, VRC-HIVDNA016-00-VP+VRC-HIVADV014-00-VP, INO-6145, JNJ-9220, gp145 C.6980; cOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, MVA-BN HIV-1 vaccine regimen, UBI HIV gp120, mRNA based prophylactic vaccines, VPI-211, and TBL-1203HI.

### D. Kits

In another aspect, this disclosure additionally provides a kit useful in performing diagnostic and prognostic assays using the antibodies, polypeptides, and nucleic acids of the present invention. Kits of the present invention include a suitable container comprising an HIV antibody, a polypeptide or a nucleic acid of the invention in either labeled or unlabeled form. In addition, when the antibody, polypeptide or nucleic acid is supplied in a labeled form suitable for an indirect binding assay, the kit further includes reagents for performing the appropriate indirect assay. For example, the kit may include one or more suitable containers, including enzyme substrates or derivatizing agents, depending on the nature of the label. Control samples and/or instructions may also be included. The present invention also provides kits for detecting the presence of the HIV antibodies or the nucleotide sequence of the HIV antibody of the present invention in a biological sample by PCR or mass spectrometry.

In some embodiments, the kit further comprises a pharmaceutically acceptable dose unit of a pharmaceutically effective amount of an anti-HIV agent. The two pharmaceutically acceptable dose units can optionally take the form of a single pharmaceutically acceptable dose unit. In some embodiments, the anti-HIV agent is one selected from the group consisting of a non-nucleoside reverse transcriptase inhibitor, a protease inhibitor, an entry or fusion inhibitor, and an integrase inhibitor.

In some embodiments, the kit also includes a container that contains the composition and optionally informational material. The informational material can be descriptive, instructional, marketing or other material that relates to the methods described herein and/or the use of the agents for therapeutic benefit. In an embodiment, the kit also includes an additional therapeutic agent, as described above. For example, the kit includes a first container that contains the composition and a second container for the additional therapeutic agent.

The informational material of the kits is not limited in its form. In some embodiments, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

The kit can include one or more containers for the composition. In some embodiments, the kit contains separate containers, dividers or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading. Such a kit may optionally contain a syringe to allow for injection of the antibody contained within the kit into an animal, such as a human.

### E. Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “antibody” (Ab) as used herein includes monoclonal antibodies, polyclonal antibodies, multispecific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2, IgG3, IgG4, IgM, IgA, IgD, IgE, and IgM); and biologically relevant fragment or specific binding member thereof, including but not limited to Fab, F(ab′)2, Fv, and scFv (single chain or related entity). It is understood in the art that an antibody is a glycoprotein comprising at least two heavy (H) chains and two light (L) chains inter-connected by disulfide bonds, or an antigen binding portion thereof. A heavy chain is comprised of a heavy chain variable region (VH) and a heavy chain constant region (CH1, CH2, and CH3). A light chain is comprised of a light chain variable region (VL) and a light chain constant region (CL). The variable regions of both the heavy and light chains comprise framework regions (FWR) and complementarity determining regions (CDR). The four FWR regions are relatively conserved while CDR regions (CDR1, CDR2, and CDR3) represent hypervariable regions and are arranged from NH2 terminus to the COOH terminus as follows: FWR1, CDR1, FWR2, CDR2, FWR3, CDR3, FWR4. The variable regions of the heavy and light chains contain a binding domain that interacts with an antigen while, depending on the isotype, the constant region(s) may mediate the binding of the immunoglobulin to host tissues or factors.

Also included in the definition of “antibody” as used herein are chimeric antibodies, humanized antibodies, and recombinant antibodies, human antibodies generated from a transgenic non-human animal, as well as antibodies selected from libraries using enrichment technologies available to the artisan.

A “neutralizing antibody” is one that neutralizes the ability of HIV to initiate and/or perpetuate an infection in a host and/or in target cells in vitro. The disclosure provides neutralizing human monoclonal antibodies, wherein the antibody recognizes an antigen from HIV, e.g., a gp 120 polypeptide. In some embodiments, a″neutralizing antibody” may inhibit the entry of HIV-1 virus, e.g., SF162 and/or JR-CSF, with a neutralization index>1.5 or >2.0 (Kostrikis L G et al., J. Virol., 70 (1): 445-458 (1996)).

In some embodiments, neutralizing antibodies are broadly neutralizing antibodies (e.g., monoclonal) that target HIV-L The term “broadly neutralizing antibodies” refers to antibodies that neutralize more than one HIV-1 virus species (from diverse clades and different strains within a clade) in a neutralization assay. A broadly neutralizing antibody may neutralize at least 2, 3, 4, 5, 6, 7, 8, 9 or more different strains of HIV-1, the strains belonging to the same or different clades. In some embodiments, a broadly neutralizing antibody may neutralize multiple HIV-1 species belonging to at least 2, 3, 4, 5, or 6 different clades. In some embodiments, the inhibitory concentration of the antibody may be less than about 0.0001 pg/mL, less than about 0.001 pg/mL, less than about 0.01 pg/mL, less than about 0.1 pg/mL, less than about 0.5 pg/mL, less than about 1.0 pg/mL, less than about 5 μg/mL, less than about 10 μg/mL, less than about 25 mg/mL, less than about 50 mg/mL, or less than about 100 mg/mL to neutralize about 50% of the input virus in the neutralization assay.

An “antibody fragment” comprises a portion of an intact antibody, such as the antigen binding or variable region of the intact antibody. Examples of antibody fragments include, but are not limited to, Fab, Fab′, F(ab′)2, and Fv fragments; diabodies; linear antibodies (see, for example, U.S. Pat. No. 5,641,870; Zapata et al., Protein Eng. 8 (10): 1057-1062 [1995]); single-chain antibody molecules; single-chani variable fragments; and multispecific antibodies formed from antibody fragments.

The term “variable” refers to the fact that certain segments of the variable (V) domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-20) 12 amino acids long. The variable regions of native heavy and light chains each comprise four FRs, largely adopting a beta sheet configuration, connected by three hypervariable regions, which form loops connecting, and in some cases forming part of, the beta sheet structure. The hypervariable regions in each chain are held together in close proximity by the FRs and, with the hypervariable regions from the other chain, contribute to the formation of the antigen-binding site of antibodies (see, for example, Kabat et al., Sequences of Proteins of Immunological Interest, 5th Ed. Public Health Service, National Institutes of Health, Bethesda, Md. (1991)).

The term “hypervariable region” as used herein refers to the amino acid residues of an antibody that are responsible for antigen binding. The hypervariable region generally comprises amino acid residues from a “complementarity determining region” (“CDR”).

The term “monoclonal antibody” as used herein refers to an antibody obtained from a population of substantially homogeneous antibodies, i.e., the individual antibodies comprising the population are identical except for possible naturally occurring mutations that may be present in minor amounts. The term “polyclonal antibody” refers to preparations that include different antibodies directed against different determinants (“epitopes”).

As used herein, a “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental animal or animal suitable as a disease model.

The term “polypeptide” is used in its conventional meaning, i.e., as a sequence of amino acids. The polypeptides are not limited to a specific length of the product. Peptides, oligopeptides, and proteins are included within the definition of polypeptide, and such terms can be used interchangeably herein unless specifically indicated otherwise. This term also includes post-expression modifications of the polypeptide, for example, glycosylations, acetylations, phosphorylations, and the like, as well as other modifications known in the art, both naturally occurring and non-naturally occurring. A polypeptide can be an entire protein, or a subsequence thereof. Particular polypeptides of interest in the context of this invention are amino acid subsequences comprising CDRs, VH, and VL, being capable of binding an antigen or HIV-infected cell.

A polypeptide “variant,” as the term is used herein, is a polypeptide that typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of techniques well known in the art.

“Homology” or “sequence identity” refers to the percentage of residues in the polynucleotide or polypeptide sequence variant that are identical to the non-variant sequence after aligning the sequences and introducing gaps, if necessary, to achieve the maximum percent homology. In particular embodiments, polynucleotide and polypeptide variants have at least about 70%, at least about 75%, at least about 80%, at least about 90%, at least about 95%, at least about 98%, or at least about 99% polynucleotide or polypeptide homology with a polynucleotide or polypeptide described herein.

Such variant polypeptide sequences will share 70% or more (i.e. 75%, 80%, 85%, 90%, 95%, 96%, 97%, 98%, 99% or more) sequence identity with the sequences recited in the application. In additional embodiments, the described invention provides polypeptide fragments comprising various lengths of contiguous stretches of amino acid sequences disclosed herein. For example, peptide sequences are provided by this invention that comprise at least about 5, 10, 15, 20, 30, 40, 50, 75, 100, 150, or more contiguous peptides of one or more of the sequences disclosed herein as well as all intermediate lengths there between.

The terms “nucleic acid” and “polynucleotide” are used interchangeably herein to refer to single-stranded or double-stranded RNA, DNA, or mixed polymers. Polynucleotides can include genomic sequences, extra-genomic and plasmid sequences, and smaller engineered gene segments that express, or can be adapted to express polypeptides.

An “isolated nucleic acid” is a nucleic acid that is substantially separated from other genome DNA sequences as well as proteins or complexes such as ribosomes and polymerases, which naturally accompany a native sequence. The term encompasses a nucleic acid sequence that has been removed from its naturally occurring environment and includes recombinant or cloned DNA isolates and chemically synthesized analogues or analogues biologically synthesized by heterologous systems. A substantially pure nucleic acid includes isolated forms of the nucleic acid. Accordingly, this refers to the nucleic acid as originally isolated and does not exclude genes or sequences later added to the isolated nucleic acid by the hand of man.

A polynucleotide “variant,” as the term is used herein, is a polynucleotide that typically differs from a polynucleotide specifically disclosed herein in one or more substitutions, deletions, additions and/or insertions. Such variants can be naturally occurring or can be synthetically generated, for example, by modifying one or more of the polynucleotide sequences of the invention and evaluating one or more biological activities of the encoded polypeptide as described herein and/or using any of a number of techniques well known in the art.

As used herein, the term “cell” can be any cell, including, but not limited to, that of a eukaryotic, multicellular species (for example, as opposed to a unicellular yeast cell), such as, but not limited to, a mammalian cell or a human cell. A cell can be present as a single entity, or can be part of a larger collection of cells. Such a “larger collection of cells” can comprise, for example, a cell culture (either mixed or pure), a tissue (for example, endothelial, epithelial, mucosa or other tissue), an organ (for example, lung, liver, muscle and other organs), an organ system (for example, circulatory system, respiratory system, gastrointestinal system, urinary system, nervous system, integumentary system or other organ system), or an organism (e.g., a bird, mammal, or the like).

“Label” as used herein refers to a detectable compound or composition that is conjugated directly or indirectly to the antibody so as to generate a “labeled” antibody. A label can also be conjugated to a polypeptide and/or a nucleic acid sequence disclosed herein. The label can be detectable by itself (for example, radioisotope labels or fluorescent labels) or, in the case of an enzymatic label, can catalyze chemical alteration of a substrate compound or composition that is detectable. Antibodies and polypeptides of the described invention also can be modified to include an epitope tag or label, for example, for use in purification or diagnostic applications. Suitable detection means include the use of labels such as, but not limited to, radionucleotides, enzymes, coenzymes, fluorescers, chemiluminescers, chromogens, enzyme substrates or co-factors, enzyme inhibitors, prosthetic group complexes, free radicals, particles, dyes, and the like.

The term “assessing” includes any form of measurement, and includes determining if an element is present or not. The terms “determining,” “measuring,” “evaluating,” “assessing,” and “assaying” are used interchangeably and include quantitative and qualitative determinations. Assessing may be relative or absolute. “Assessing the presence of” includes determining the amount of something present, and/or determining whether it is present or absent. As used herein, the terms “determining,” “measuring,” and “assessing” and “assaying” are used interchangeably and include both quantitative and qualitative determinations.

The terms “treating” or “treatment” or “alleviation” are used interchangeably and refer to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for an infection if, after receiving a therapeutic amount of an antibody according to the methods of the present invention, the patient shows observable and/or measurable reduction in or absence of one or more of the following: reduction in the number of infected cells or absence of the infected cells; reduction in the percent of total cells that are infected; and/or relief to some extent, one or more of the symptoms associated with the specific infection; reduced morbidity and mortality, and improvement in quality of life issues. The above parameters for assessing successful treatment and improvement in the disease are readily measurable by routine procedures familiar to a physician.

An “effective amount” or “therapeutically effective amount” refers to an amount of the compound or agent that is capable of producing a medically desirable result in a treated subject. The treatment method can be performed in vivo or ex vivo, alone or in conjunction with other drugs or therapy. A therapeutically effective amount can be administered in one or more administrations, applications or dosages and is not intended to be limited to a particular formulation or administration route.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “decrease,” “reduced,” “reduction,” “decrease,” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “modulate” is meant to refer to any change in biological state, i.e., increasing, decreasing, and the like.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

“Combination” therapy, as used herein, unless otherwise clear from the context, is meant to encompass administration of two or more therapeutic agents in a coordinated fashion, and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423. Administration “in combination with” one or more further therapeutic agents includes simultaneous (concurrent) and consecutive administration in any order.

“Sample,” “test sample,” and “patient sample” may be used interchangeably herein. The sample can be a sample of, serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

The terms “inhibit” and “antagonize,” as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down-regulate a protein, a gene, and an mRNA stability, expression, function and activity, e.g., antagonists.

“Parenteral” administration of a composition includes, e.g., subcutaneous (s.c.), intravenous (i.v.), intramuscular (i.m.), or intrasternal injection, or infusion techniques.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism.

Multiple techniques of administering a compound exist in the art, including, but not limited to, intravenous, oral, aerosol, parenteral, ophthalmic, pulmonary, and topical administration.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### F. Examples

### Example 1

This example describes the materials and methods used in the subsequent EXAMPLES below.

Polyreactivity Assays: Polyreactivity assays were conducted using ELISA detection of non-specific binding to baculovirus extracts as described (Hötzel et al. 2012, mAbs, 4:6, 753-760). Briefly, a solution of 1% baculovirus particles in 100 mM sodium bicarbonate buffer pH 9.6 was absorbed onto the wells of a 384-well ELISA plate (Nunc Maxisorp) using a Tecan Freedom Evo liquid handling robot, and the plate was incubated overnight at 4° C. The plate was blocked with 0.5% BSA in PBS for 1 hour at room temperature. Purified IgGs (diluted to 1 μg/mL in PBS, 0.5% BSA) were added to the blocked assay plate and incubated for 3 hours at room temperature. Bound IgG was detected as the luminescence signal at 425 nm using an HRP-conjugated anti-human IgG (H&L) secondary antibody (Genscript) and SuperSignal ELISA Femto Maximum Sensitivity Substrate (Thermo Fisher Scientific).

Neutralization Assays: HIV neutralization potency was determined using a luciferase-based TZM-bl assay as previously described (Montefiori, D. C. 1019, Methods Mol. Biol. 485, 395-405).

Non-human primate half-life: Half-life in macaques was determined as described (Shingai, M. et al., 2014, J. Exp. Med. 211, 2061-74). Briefly, macaques were administered 10 mg/kg of purified IgG and antibody was detected in serum over 70 days by ELISA and/or neutralization activity. Half-life was determined by fitting data from days 8-70.

Mouse Pharmacokinetics: Engineered reagents were radiolabeled with ¹²⁵I using the iodgen method and purified by SEC (Yazaki P J et al., 2008, Nucl Med Biol., 35 (2), 151-8). Mouse half-life and tissue distribution were determined as previously described (Yazaki P J et al., 2013, PEDS 26 (3), 187-193). Briefly, NSG mice were injected with mouse IVIG (4 hours before) then with ˜3 μg of labeled Ab via tail vein injection (˜3-6 micro-curies). Blood samples were taken counted for radio labeled protein. Those data are plotted as a function of time for blood clearance. The mice were sacrificed at 96 hours and the organs and carcass were weighed and counted and the counts normalized to weight and plotted.

### Example 2

The parent antibody NIH45-46 G54W consists of the NIH45-46 HC with the G54W mutation paired with the NIH45-46 LC. The new antibody variants have mutations on one or both of these chains.

The single chains from which the new antibody variants are constructed are:

Chain name description (mutations listed
with standard Kabat numbering)

| | |
|---|---|
| HC1 HC | NIH45-46 G54W HC with T68D |
| SAP10 LC | NIH45-46 LC with Q27E, S28H, and S30D |
| SAP10T LC | NIH45-46 LC with Q27E, S28H, S30D, and S74T |
| SAP10T-LS LC | NIH45-46 LC with Q27E, S28H, S30D, S74T, |
| | M428L, and N434S |
| SAP3 LC | NIH45-46 LC with Q27E, S28Y, and S30D |
| SAP8 LC | NIH45-46 LC with Q27D, S28H |
| m2 LC | NIH45-46 LC with S28Y |

The new antibody reagents consist of the following HC/LC pairs (LS designates previously described mutations in the Fc region, e.g., M428L/N434S):
HCl/SAP10
HCl/SAP10-LS
HCl/SAP10T
HCl/SAP10T-LS
HCl/SAP8
HCl/SAP3
HCl/NIH45-46 LC
HCl/m2
NIH45-46 G54W/SAP10
NIH45-46 G54W/SAP3
NIH45-46 G54W/SAP8

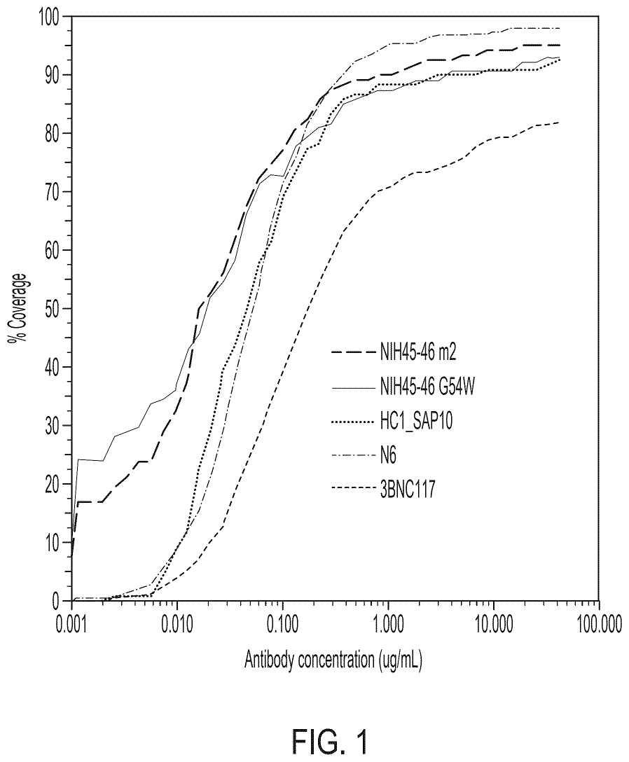

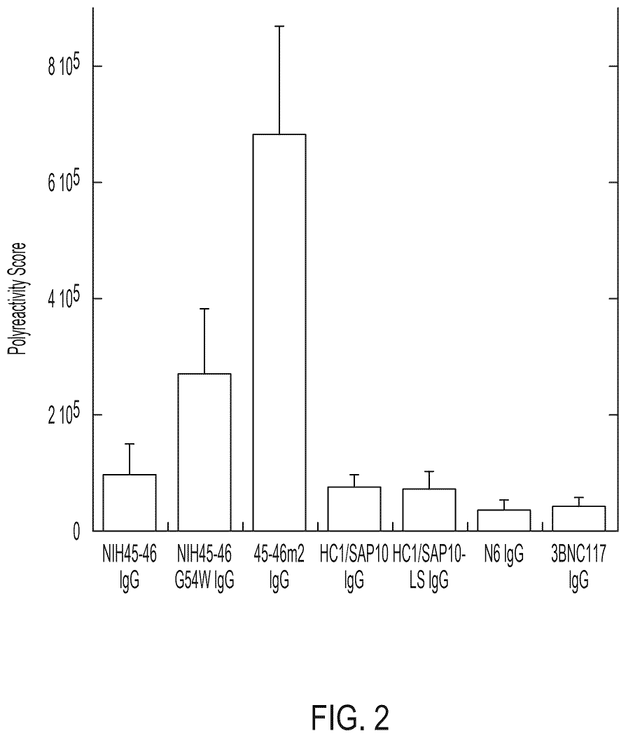

G54W mutants of NIH45-46 (including NIH45-46 G54W and NIH45-46 m2) have short in vivo half-lives (Shingai, M. et al., 2014, J. Exp. Med. 211, 2061-74). Short half-lives correlate with high levels of polyreactivity (Hotzel, I., et al., 2012, mAbs 4, 753-760). Our new engineered variant HCl/SAP10 shows reduced polyreactivity (FIG. 2) and retains potency (FIG. 1, Tables 1 and 2). Although the neutralization potency of HCl/SAP10 is slightly reduced compared to NIH45-46 G54W and NIH45-46 m2, it still compares favorably with other anti-HIV bNAbs, such as N6 and 3BNC117 (FIG. 1).

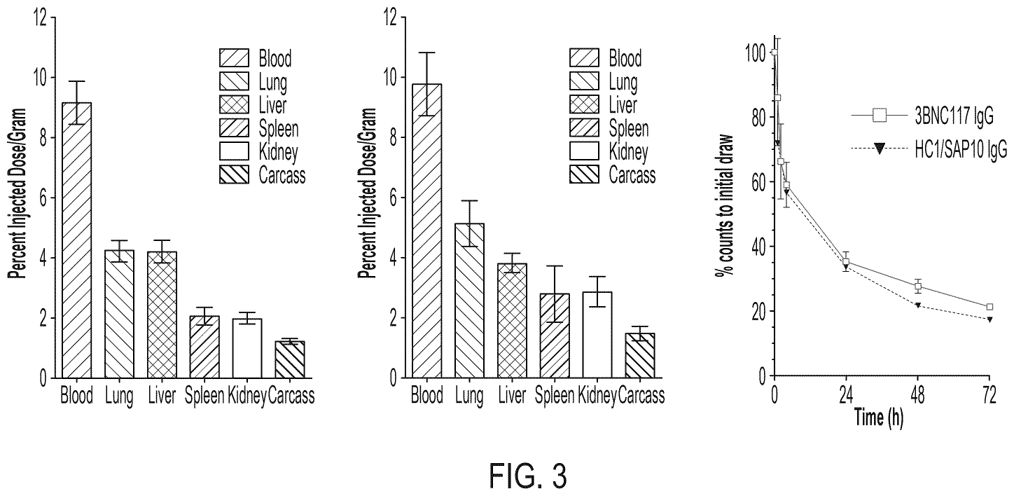

The half-life of bNAb NIH45-46 m2 in non-human primates (NHPs) could not be determined due to quick clearance (Shingai, M. et al., 2014, J. Exp. Med. 211, 2061-74). In contrast, our new variant HCl/SAP10-LS has a half-life and tissue distribution similar to 3BNC117 in mice (FIG. 3) and a half-life in NHPs of 8.4 days.

The neutralization data for the new reagents are shown in Table 1 and summarized in Table 2.

TABLE 1
Neutralization IC50s (μg/mL) of NIH45-46 variants in TZM-bl assays.

| | | NIH45- | NIH45- | | | |
|---|---|---|---|---|---|---|
| Virus ID | Clade | 46 G54W | 46 m2 | HC1/SAP3 | HC1/SAP8 | HC1/SAP10 |
| 6535.3 | B | 0.041 | 0.033 | 0.17 | 0.113 | 0.111 |
| QH0692.42 | B | 0.233 | 0.279 | 0.961 | 0.345 | 0.286 |
| SC422661.8 | B | 0.013 | 0.013 | 0.064 | 0.027 | 0.028 |
| PVO.4 | B | 0.06 | 0.044 | 0.216 | 0.109 | 0.082 |
| TRO.11 | B | 0.027 | 0.026 | 0.057 | 0.083 | 0.052 |
| AC10.0.29 | B | 0.184 | 0.102 | 0.222 | 0.155 | 0.154 |
| RHPA4259.7 | B | 0.007 | 0.006 | 0.028 | 0.011 | 0.011 |
| THRO4156.18 | B | 0.434 | 0.53 | 1.008 | 0.495 | 0.456 |
| REJO4541.67 | B | 0.004 | 0.003 | 0.012 | 0.01 | 0.015 |
| TRJO4551.58 | B | 0.019 | 0.018 | 0.038 | 0.016 | 0.009 |
| WITO4160.33 | B | 0.021 | 0.011 | 0.036 | 0.045 | 0.038 |
| CAAN5342.A2 | B | 0.129 | 0.148 | 0.759 | 0.338 | 0.373 |
| WEAU_d15_410_787 | B (T/F) | 0.006 | 0.005 | 0.027 | 0.007 | 0.006 |
| 1006_11_C3_1601 | B | 0.009 | 0.004 | 0.02 | 0.017 | 0.01 |
| 1054_07_TC4_1499 | B (T/F) | 0.079 | 0.051 | 0.613 | 0.206 | 0.256 |
| 1056_10_TA11_1826 | B (T/F) | 0.055 | 0.018 | 0.108 | 0.118 | 0.057 |
| 1012_11_TC21_3257 | B (T/F) | 0.005 | 0.003 | 0.03 | 0.013 | 0.014 |
| 6240_08_TA5_4622 | B (T/F) | 0.126 | 0.129 | 0.508 | 0.215 | 0.121 |
| 6244_13_B5_4576 | B | 0.032 | 0.032 | 0.18 | 0.051 | 0.054 |
| 62357_14_D3_4589 | B (T/F) | 0.024 | 0.013 | 0.041 | 0.033 | 0.024 |
| SC05_8C11_2344 | B | 0.06 | 0.059 | 0.449 | 0.12 | 0.112 |
| Du156.12 | C | 0.009 | 0.01 | 0.064 | 0.022 | 0.021 |
| Du172.17 | C | 2.101 | 0.023 | 0.06 | 0.79 | 0.15 |
| Du422.1 | C | 43.262 | 3.332 | 50 | 50 | 50 |
| ZM197M.PB7 | C | 0.072 | 0.083 | 0.26 | 0.065 | 0.077 |
| ZM214M.PL15 | C | 0.04 | 0.021 | 0.063 | 0.074 | 0.076 |
| ZM233M.PB6 | C | 0.04 | 0.017 | 0.146 | 0.128 | 0.082 |
| ZM249M.PL1 | C | 0.012 | 0.012 | 0.044 | 0.022 | 0.017 |
| ZM53M.PB12 | C | 0.076 | 0.098 | 0.332 | 0.132 | 0.09 |
| ZM109F.PB4 | C | 0.047 | 0.06 | 0.292 | 0.136 | 0.081 |
| ZM135M.PL10a | C | 0.011 | 0.01 | 0.038 | 0.02 | 0.015 |
| CAP45.2.00.G3 | C | 8.359 | 0.014 | 0.021 | 5.09 | 0.332 |
| CAP210.2.00.E8 | C | 21.882 | 1.802 | 6.761 | 50 | 26.231 |
| HIV-001428-2.42 | C | 0.002 | 0.002 | 0.005 | 0.002 | 0.002 |
| HIV-0013095-2.11 | C | 0.01 | 0.011 | 0.053 | 0.014 | 0.018 |
| HIV-16055-2.3 | C | 0.01 | 0.011 | 0.058 | 0.019 | 0.016 |
| HIV-16845-2.22 | C | 0.244 | 0.16 | 0.836 | 0.395 | 0.263 |
| Ce1086_B2 | C | 0.007 | 0.011 | 0.063 | 0.047 | 0.022 |
| Ce0393_C3 | C | 0.03 | 0.024 | 0.091 | 0.098 | 0.062 |
| Ce1176_A3 | C | 0.094 | 0.094 | 0.398 | 0.142 | 0.15 |
| Ce2010_F5 | C | 0.039 | 0.035 | 0.074 | 0.05 | 0.04 |
| Ce0682_E4 | C | 0.014 | 0.008 | 0.045 | 0.022 | 0.025 |
| Ce1172_H1 | C | 50 | NT | 50 | 50 | 50 |
| Ce2060_G9 | C | 0.015 | 0.016 | 0.062 | 0.035 | 0.04 |
| Ce703010054_2A2 | C | 0.022 | 0.019 | 0.073 | 0.028 | 0.029 |
| BF1266.431a | C | 0.005 | 0.004 | 0.029 | 0.01 | 0.008 |
| 246F C1G | C | 0.608 | 0.107 | 0.408 | 2.276 | 0.685 |
| 249M B10 | C | 0.011 | 0.01 | 0.043 | 0.014 | 0.015 |
| ZM247v1(Rev-) | C | 0.31 | 0.009 | 0.047 | 0.537 | 0.101 |
| 7030102001E5(Rev-) | C (T/F) | 0.047 | 0.047 | 0.24 | 0.085 | 0.079 |
| 1394C9G1(Rev-) | C | 0.008 | 0.009 | 0.074 | 0.025 | 0.016 |
| Ce704809221_1B3 | C | 0.061 | 0.073 | 0.195 | 0.098 | 0.095 |
| CNE19 | BC | 0.014 | 0.015 | 0.04 | 0.019 | 0.016 |
| CNE20 | BC | 0.026 | 0.007 | 0.039 | 0.044 | 0.027 |
| CNE21 | BC | 0.015 | 0.01 | 0.067 | 0.056 | 0.03 |
| CNE17 | BC | 0.042 | 0.052 | 0.181 | 0.061 | 0.042 |
| CNE30 | BC | 0.095 | 0.105 | 0.401 | 0.215 | 0.135 |
| CNE52 | BC | 0.007 | 0.007 | 0.017 | 0.014 | 0.009 |
| CNE53 | BC | 0.005 | 0.007 | 0.02 | 0.007 | 0.006 |
| CNE58 | BC | 0.014 | 0.013 | 0.034 | 0.022 | 0.016 |
| MS208.A1 | A | 0.023 | 0.026 | 0.061 | 0.042 | 0.045 |
| Q23.17 | A | 0.007 | 0.004 | 0.015 | 0.013 | 0.01 |
| Q461.e2 | A | 0.055 | 0.05 | 0.283 | 0.08 | 0.103 |
| Q769.d22 | A | 0.009 | 0.005 | 0.009 | 0.008 | 0.009 |
| Q259.d2.17 | A | 0.007 | 0.006 | 0.051 | 0.019 | 0.02 |
| Q842.d12 | A | 0.009 | 0.007 | 0.028 | 0.013 | 0.013 |
| 0260.v5.c36 | A | 0.072 | 0.086 | 0.376 | 0.159 | 0.134 |
| 3415.v1.c1 | A | 0.012 | 0.018 | 0.086 | 0.029 | 0.024 |
| 3365.v2.c2 | A | 0.013 | 0.015 | 0.057 | 0.024 | 0.017 |
| 191955_A11 | A (T/F) | 0.027 | 0.02 | 0.109 | 0.046 | 0.04 |
| 191084 B7-19 | A (T/F) | 0.011 | 0.013 | 0.038 | 0.015 | 0.01 |
| 9004SS_A3_4 | A (T/F) | 0.017 | 0.025 | 0.073 | 0.047 | 0.023 |
| T257-31 | CRF02_A | 0.056 | 0.109 | 0.484 | 0.122 | 0.082 |
| 928-28 | CRF02_A | 0.036 | 0.042 | 0.133 | 0.089 | 0.056 |
| 263-8 | CRF02_A | 0.021 | 0.023 | 0.085 | 0.039 | 0.019 |
| T250-4 | CRF02_A | 3.465 | 2.094 | 5.888 | 50 | 50 |
| T251-18 | CRF02_A | 0.161 | 0.144 | 0.811 | 0.333 | 0.315 |
| T278-50 | CRF02_A | 50 | NT | 50 | 50 | 50 |
| T255-34 | CRF02_A | 0.033 | 0.051 | 0.132 | 0.08 | 0.027 |
| 211-9 | CRF02_A | 12.214 | 0.245 | 0.806 | >50 | 6.753 |
| 235-47 | CRF02_A | 0.009 | 0.01 | 0.04 | 0.02 | 0.015 |
| 620345.c01 | CRF01_AE | 50 | NT | 50 | 50 | 50 |
| CNE8 | CRF01_AE | 0.031 | 0.053 | 0.228 | 0.051 | 0.049 |
| C1080.c03 | CRF01_AE | 0.11 | 0.086 | 0.348 | 0.205 | 0.212 |
| R2184.c04 | CRF01_AE | 0.024 | 0.021 | 0.047 | 0.024 | 0.015 |
| R1166.c01 | CRF01_AE | 0.268 | 0.249 | 0.843 | 0.368 | 0.262 |
| C2101.c01 | CRF01_AE | 0.034 | 0.018 | 0.057 | 0.048 | 0.069 |
| C3347.c11 | CRF01_AE | 0.01 | 0.006 | 0.042 | 0.023 | 0.026 |
| C4118.c09 | CRF01_AE | 0.026 | 0.012 | 0.059 | 0.038 | 0.045 |
| CNE5 | CRF01_AE | 0.029 | 0.035 | 0.16 | 0.061 | 0.052 |
| BJOX009000.02. | CRF01_AE | 0.255 | 0.348 | 1.754 | 0.721 | 0.756 |
| BJOX015000.11. | CRF01_AE | 0.092 | 0.043 | 0.259 | 0.176 | 0.27 |
| BJOX010000.06. | CRF01_AE | 1.149 | 1.377 | 8.658 | 3.442 | 2.39 |
| BJOX025000.01. | CRF01_AE | 0.015 | 0.005 | 0.012 | 0.023 | 0.027 |
| BJOX028000.10. | CRF01_AE | 0.002 | 0.001 | 0.004 | 0.01 | 0.007 |
| X1193_c1 | G | 0.012 | 0.009 | 0.026 | 0.016 | 0.018 |
| P0402_c2_11 | G | 0.006 | 0.01 | 0.047 | 0.016 | 0.018 |
| X1254_c3 | G | 0.022 | 0.023 | 0.078 | 0.028 | 0.028 |
| X2088_c9 | G | 50 | NT | 50 | 50 | 50 |
| X2131_C1_B5 | G | 0.058 | 0.047 | 0.234 | 0.081 | 0.049 |
| P1981_C5_3 | G | 0.037 | 0.04 | 0.159 | 0.065 | 0.051 |
| X1632_S2_B10 | G | 0.007 | 0.004 | 0.036 | 0.01 | 0.016 |
| 3016.v5.c45 | D | 0.651 | 0.018 | 0.099 | 44.378 | 2.073 |
| A07412M1.vrc12 | D | 0.026 | 0.024 | 0.103 | 0.036 | 0.027 |
| 231965.c01 | D | 0.024 | 0.023 | 0.13 | 0.049 | 0.058 |
| 231966.c02 | D | 0.022 | 0.012 | 0.084 | 0.053 | 0.032 |
| 191821_E6_1 | D (T/F) | 0.042 | 0.055 | 0.265 | 0.102 | 0.094 |
| 3817.v2.c59 | CD | 50 | 0.939 | 50 | 50 | 50 |
| 6480.v4.c25 | CD | 0.015 | 0.012 | 0.037 | 0.02 | 0.021 |
| 6952.v1.c20 | CD | 0.019 | 0.018 | 0.048 | 0.016 | 0.016 |
| 6811.v7.c18 | CD | 0.021 | 0.023 | 0.166 | 0.042 | 0.051 |
| 89-F1_2_25 | CD | 50 | NT | 13.478 | 50 | 50 |
| 3301.v1.c24 | AC | 0.009 | 0.008 | 0.044 | 0.015 | 0.02 |
| 6041.v3.c23 | AC | 0.002 | 0.002 | 0.021 | 0.006 | 0.007 |
| 6540.v4.c1 | AC | 44.909 | NT | 28.498 | 50 | 50 |
| 6545.v4.c1 | AC | 6.643 | 8.672 | 14.416 | 5.305 | 37.87 |
| 0815.v3.c3 | ACD | 0.004 | 0.004 | 0.012 | 0.01 | 0.009 |
| 3103.v3.c10 | ACD | 0.305 | 0.25 | 0.534 | 0.366 | 0.227 |

| | | | | | |
|---|---|---|---|---|---|
| ADA_MM (Martin SHIV Env | 0.075 | 0.073 | 0.265 | 0.129 | 0.1 |

TABLE 2
Summary of neutralization potency of NIH45-46 variant bNAbs.

| | Geometric | Fold worse | Fold worse |
|---|---|---|---|
| | Mean | than | than |
| NIH45-46 G54W | IC50 (μg/mL) | NIH45-46 G54W | NIH45-46 m2 |
| | 0.06 | — | 2.13 |
| NIH45-46 m2 | 0.028 | 0.47 | — |
| HC1/SAP3 | 0.158 | 2.65 | 5.65 |
| HC1/SAP8 | 0.104 | 1.74 | 3.7 |
| HC1/SAP10 | 0.087 | 1.45 | 3.1 |

What is claimed is:

1. An isolated nucleic acid molecule encoding an anti-HIV antibody or antigen binding portion thereof comprising:
(i) a set of heavy chain complementarity determining regions (HCDR1, HCDR2, and HCDR3) contained in SEQ ID NO: 4; and
(ii) a set of light chain complementarity determining regions (LCDR1, LCDR2, and LCDR3) contained in SEQ ID NO: 36.

2. The isolated nucleic acid molecule of claim 1, wherein the anti-HIV-1 antibody is a scFv antibody, a chimeric antibody, a humanized antibody, or a monoclonal antibody.

3. A vector comprising the nucleic acid molecule of claim 1, wherein the vector is a plasmid, a phage vector, a virral vector, or a cosmid.

4. A cultured cell comprising the vector of claim 3, wherein the cultured cell is a human embryonic kidney (HEK) cell, a COS-7 cell, a baby hamster kidney (BHK) cell, a TM4 cell, a CV1 cell, a VERO-76 cell, a HeLa cell, a Madin-Darby canine kidney (MDCK) cell, a W138 cell, a Hep G2 cell, a MMT 060562 cell, a TRI cell, a MRC 5 cell, an FS4 cell, a myeloma cell, or a Chinese hamster ovary (CHO) cell.

5. A method for making an anti-HIV antibody or a fragment thereof, comprising:
obtaining the cultured cell of claim 4;
culturing the cell in a medium under conditions permitting expression of a polypeptide encoded by the vector and assembling of an antibody or fragment thereof; and purifying the antibody or fragment from the cultured cell or the medium.

6. An isolated nucleic acid molecule encoding an anti-HIV antibody or antigen binding portion thereof comprising:
(i) a set of heavy chain complementarity determining regions (HCDR1, HCDR2, and HCDR3) comprising the respective sequences of SEQ ID NOs: 81, 110, and 87; and
(ii) a set of light chain complementarity determining regions (LCDR1, LCDR2, and LCDR3) comprising the respective sequences of SEQ ID NOs: 100, 93, and 96.

7. The isolated nucleic acid molecule of claim 6, wherein the anti-HIV-1 antibody is a scFv antibody, a chimeric antibody, a humanized antibody, or a monoclonal antibody.

8. A vector comprising the nucleic acid molecule of claim 6, wherein the vector is a plasmid, a phage vector, a virral vector, or a cosmid.

9. A cultured cell comprising the vector of claim 8, wherein the cultured cell is a human embryonic kidney (HEK) cell, a COS-7 cell, a baby hamster kidney (BHK) cell, a TM4 cell, a CV1 cell, a VERO-76 cell, a HeLa cell, a Madin-Darby canine kidney (MDCK) cell, a W138 cell, a Hep G2 cell, a MMT 060562 cell, a TRI cell, a MRC 5 cell, an FS4 cell, a myeloma cell, or a Chinese hamster ovary (CHO) cell.

10. A method for making an anti-HIV antibody or a fragment thereof, comprising:
obtaining the cultured cell of claim 9;
culturing the cell in a medium under conditions permitting expression of a polypeptide encoded by the vector and assembling of an antibody or fragment thereof; and purifying the antibody or fragment from the cultured cell or the medium.


SEQUENCE LISTING (112 sequences)

SEQ ID NO: 1 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 455 aa, synthetic construct
  REGION 1..455: note = Synthetic
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVDMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVEMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMDRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMERDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 455 aa, synthetic construct
  REGION 1..455: note = Synthetic
QVRLSQSGGQMKKPGESMRLSCRASGYEFLNCPINWIRLAPGRRPEWMGWLKPRWGAVNYARKFQGRVTMTRDVYDDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 455 aa, synthetic construct
  REGION 1..455: note = Synthetic
QVRLSQSGGQMKKPGESMRLSCRASGYEFLNCPINWIRLAPGRRPEWMGWLKPRWGAVNYARKFQGRVTMTRDVYEDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 455 aa, synthetic construct
  REGION 1..455: note = Synthetic
QVRLSQSGGQMKKPGESMRLSCRASGYEFLNCPINWIRLAPGRRPEWMGWLKPRWGAVNYARKFQGRVTMTRDVYSDTAFLELRDLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 455 aa, synthetic construct
  REGION 1..455: note = Synthetic
QVRLSQSGGQMKKPGESMRLSCRASGYEFLNCPINWIRLAPGRRPEWMGWLKPRWGAVNYARKFQGRVTMTRDVYSDTAFLELRELTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 455 aa, synthetic construct
  REGION 1..455: note = Synthetic
QVRLSQSGGQMKKPGESMRLSCRASGYEFLNCPINWIRLAPGRRPEWMGWLKPRWGAVNYARKFQGRVTMTRDVYSDTAFLELRSLTSEDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 455 aa, synthetic construct
  REGION 1..455: note = Synthetic
QVRLSQSGGQMKKPGESMRLSCRASGYEFLNCPINWIRLAPGRRPEWMGWLKPRWGAVNYARKFQGRVTMTRDVYSDTAFLELRSLTSDDTADYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 455 aa, synthetic construct
  REGION 1..455: note = Synthetic
QVRLSQSGGQMKKPGESMRLSCRASGYEFLNCPINWIRLAPGRRPEWMGWLKPRWGAVNYARKFQGRVTMTRDVYSDTAFLELRSLTSDDTAEYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 455 aa, synthetic construct
  REGION 1..455: note = Synthetic
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGADVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 455 aa, synthetic construct
  REGION 1..455: note = Synthetic
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAEVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 455 aa, synthetic construct
  REGION 1..455: note = Synthetic
QVRLSQSGGQMKKPGESMRLDCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 455 aa, synthetic construct
  REGION 1..455: note = Synthetic
QVRLSQSGGQMKKPGESMRLECRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 455 aa, synthetic construct
  REGION 1..455: note = Synthetic
QVRLSQSGGQMKKPGESMRLSCRASGYEFLNCPINWIRLAPGRRPEWMGWLKPRGDAVNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 455 aa, synthetic construct; identical to SEQ ID NO: 19
  REGION 1..455: note = Synthetic

SEQ ID NO: 21 - protein, 455 aa, synthetic construct
  REGION 1..455: note = Synthetic
QVRLSQSGGQMKKPGESMRLSCRASGYEFLNCPINWIRLAPGRRPEWMGWLKPRGGADNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 455 aa, synthetic construct
  REGION 1..455: note = Synthetic
QVRLSQSGGQMKKPGESMRLSCRASGYEFLNCPINWIRLAPGRRPEWMGWLKPRGGAENYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMTRDDYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMTRDEYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMTRDVYSDTADLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMTRDVYSDTAELELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMTRDVYSDTAYLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 455 aa, synthetic construct; identical to SEQ ID NO: 27
  REGION 1..455: note = Synthetic

SEQ ID NO: 29 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKDCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKECTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 455 aa, synthetic construct
  REGION 1..455: note = Synthetic
QVRLDQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 455 aa, synthetic construct
  REGION 1..455: note = Synthetic
QVRLEQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 34 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVDMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 35 - protein, 455 aa, synthetic construct
  REGION 1..455: note = Synthetic
QVRLSQSGGQMKKPGESMRLSCRASGYEFLNCPINWIRLAPGRRPEWMGWLKPRGGAVNYARKFQGRVEMTRDVYSDTAFLELRSLTSDDTAVYFCTRGKYCTARDYYNWDFEHWGRGAPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 36 - protein, 210 aa, synthetic construct
  REGION 1..210: note = Synthetic
EIVLTQSPATLSLSPGETAIISCRTSEHGDLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 210 aa, synthetic construct
  REGION 1..210: note = Synthetic
EIVLTQSPATLSLSPGETAIISCRTSEHGDLAWYQQRPGQAPRLVIYSGSTRAAGIPDRFSGSRWGADYNLSITNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 210 aa, synthetic construct
  REGION 1..210: note = Synthetic
EIVLTQSPATLSLSPGETAIISCRTSEYGDLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 210 aa, synthetic construct
  REGION 1..210: note = Synthetic
EIVLTQSPATLSLSPGETAIISCRTSDHGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 210 aa, synthetic construct
  REGION 1..210: note = Synthetic
EIVLTQSPATLSLSPGETAIISCRTSEHGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 210 aa, synthetic construct
  REGION 1..210: note = Synthetic
EIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 210 aa, synthetic construct
  REGION 1..210: note = Synthetic
EIVLDQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 210 aa, synthetic construct
  REGION 1..210: note = Synthetic
EIVLEQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 210 aa, synthetic construct
  REGION 1..210: note = Synthetic
EIVLTQDPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 210 aa, synthetic construct
  REGION 1..210: note = Synthetic
EIVLTQEPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 210 aa, synthetic construct
  REGION 1..210: note = Synthetic
EIVLTQSPADLSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 210 aa, synthetic construct
  REGION 1..210: note = Synthetic
EIVLTQSPAELSLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 210 aa, synthetic construct
  REGION 1..210: note = Synthetic
EIVLTQSPATLDLSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 210 aa, synthetic construct
  REGION 1..210: note = Synthetic
EIVLTQSPATLELSPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 210 aa, synthetic construct
  REGION 1..210: note = Synthetic
EIVLTQSPATLSLDPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 210 aa, synthetic construct
  REGION 1..210: note = Synthetic
EIVLTQSPATLSLEPGETAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 210 aa, synthetic construct
  REGION 1..210: note = Synthetic
EIVLTQSPATLSLSPGEDAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 210 aa, synthetic construct
  REGION 1..210: note = Synthetic
EIVLTQSPATLSLSPGEEAIISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 210 aa, synthetic construct
  REGION 1..210: note = Synthetic
EIVLTQSPATLSLSPGETADISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 210 aa, synthetic construct
  REGION 1..210: note = Synthetic
EIVLTQSPATLSLSPGETAEISCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 210 aa, synthetic construct
  REGION 1..210: note = Synthetic
EIVLTQSPATLSLSPGETAIIDCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 210 aa, synthetic construct
  REGION 1..210: note = Synthetic
EIVLTQSPATLSLSPGETAIIECRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 210 aa, synthetic construct
  REGION 1..210: note = Synthetic
EIVLTQSPATLSLSPGETAIISCDTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 210 aa, synthetic construct
  REGION 1..210: note = Synthetic
EIVLTQSPATLSLSPGETAIISCETSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDDFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDEFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFDGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFEGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGDRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGERWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRYGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRHGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRDGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSREGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRWGAEYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRWGADYDLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRWGADYELSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRWGADYNLDISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRWGADYNLEISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRWGADYNLSIDNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRWGADYNLSIENLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRWGADYNLSISNLEDGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFSGSRWGADYNLSISNLEEGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 210 aa, synthetic construct
  REGION 1..210: note = Synthetic
EIVLDQSPADLDLDPGETADIDCRTSQSGSLAWYQQRPGQAPRLVIYSGSTRAAGIPDRFSGSRWGADYNLSISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 210 aa, synthetic construct
  REGION 1..210: note = Synthetic
EIVLTQSPATLSLSPGETAIISCRTSQSGSLAWYQQRPGQAPRLVIYSGSTRAAGIPDRFDGDRDGADYDLDIDNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 5 aa, synthetic construct
  REGION 1..5: note = Synthetic
NCPIN

SEQ ID NO: 82 - protein, 8 aa, synthetic construct
  REGION 1..8: note = Synthetic
GYEFLNCP

SEQ ID NO: 83 - protein, 6 aa, synthetic construct
  REGION 1..6: note = Synthetic
GYEFLN

SEQ ID NO: 84 - protein, 17 aa, synthetic construct
  REGION 1..17: note = Synthetic
WLKPRWGAVNYARKFQG

SEQ ID NO: 85 - protein, 8 aa, synthetic construct
  REGION 1..8: note = Synthetic
LKPRWGAV

SEQ ID NO: 86 - protein, 6 aa, synthetic construct
  REGION 1..6: note = Synthetic
KPRWGA

SEQ ID NO: 87 - protein, 16 aa, synthetic construct
  REGION 1..16: note = Synthetic
GKYCTARDYYNWDFEH

SEQ ID NO: 88 - protein, 18 aa, synthetic construct
  REGION 1..18: note = Synthetic
TRGKYCTARDYYNWDFEH

SEQ ID NO: 89 - protein, 16 aa, synthetic construct
  REGION 1..16: note = Synthetic
GKYCTARDYYNWDFEH

SEQ ID NO: 90 - protein, 9 aa, synthetic construct
  REGION 1..9: note = Synthetic
RTSQSGSLA

SEQ ID NO: 91 - protein, 4 aa, synthetic construct
  REGION 1..4: note = Synthetic
QYGS

SEQ ID NO: 92 - protein, 9 aa, synthetic construct
  REGION 1..9: note = Synthetic
RTSQYGSLA

SEQ ID NO: 93 - protein, 7 aa, synthetic construct
  REGION 1..7: note = Synthetic
SGSTRAA

SEQ ID NO: 94 - skipped

SEQ ID NO: 95 - protein, 7 aa, synthetic construct
  REGION 1..7: note = Synthetic
SGSTRAA

SEQ ID NO: 96 - protein, 5 aa, synthetic construct
  REGION 1..5: note = Synthetic
QQYEF

SEQ ID NO: 97 - protein, 5 aa, synthetic construct
  REGION 1..5: note = Synthetic
QQYEF

SEQ ID NO: 98 - protein, 5 aa, synthetic construct
  REGION 1..5: note = Synthetic
QQYEF

SEQ ID NO: 99 - protein, 9 aa, synthetic construct
  REGION 1..9: note = Synthetic
RTSQYGSLA

SEQ ID NO: 100 - protein, 9 aa, synthetic construct
  REGION 1..9: note = Synthetic
RTSEHGDLA

SEQ ID NO: 101 - protein, 9 aa, synthetic construct
  REGION 1..9: note = Synthetic
RTSEYGDLA

SEQ ID NO: 102 - protein, 9 aa, synthetic construct
  REGION 1..9: note = Synthetic
RTSDHGSLA

SEQ ID NO: 103 - DNA, 1365 bp, synthetic construct
  misc_feature 1..1365: note = Synthetic
  source 1..1365: mol_type = other DNA
caagtgcgactgtcgcagtctggaggtcagatgaagaagcctggcgagtcgatgagactttcctgtcgggcttccggatatgaatttctgaattgtccaataaattggattcgcctggcccccggaagacggcctgagtggatgggatggctgaagcctaggggaggggccgtcaattacgcacgtaaatttcagggcagagtgaccatgactcgagacgtgtattccgacacagcctttttggagttgcgctccttgacatcagacgacacggccgtctatttttgtactaggggaaaatattgtactgcgcgcgactattataattgggacttcgaacactggggccggggtgccccggtcaccgtctcatcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 104 - DNA, 1365 bp, synthetic construct
  misc_feature 1..1365: note = Synthetic
  source 1..1365: mol_type = other DNA
caagtgcgactgtcgcagtctggaggtcagatgaagaagcctggcgagtcgatgagactttcctgtcgggcttccggatatgaatttctgaattgtccaataaattggattcgcctggcccccggaagacggcctgagtggatgggatggctgaagcctaggtggggggccgtcaattacgcacgtaaatttcagggcagagtgaccatgactcgagacgtgtattccgacacagcctttttggagttgcgctccttgacatcagacgacacggccgtctatttttgtactaggggaaaatattgtactgcgcgcgactattataattgggacttcgaacactggggccggggtgccccggtcaccgtctcatcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 105 - DNA, 1365 bp, synthetic construct
  misc_feature 1..1365: note = Synthetic
  source 1..1365: mol_type = other DNA
caagtgcgactgtcgcagtctggaggtcagatgaagaagcctggcgagtcgatgagactttcctgtcgggcttccggatatgaatttctgaattgtccaataaattggattcgcctggcccccggaagacggcctgagtggatgggatggctgaagcctaggtggggggccgtcaattacgcacgtaaatttcagggcagagtggacatgactcgagacgtgtattccgacacagcctttttggagttgcgctccttgacatcagacgacacggccgtctatttttgtactaggggaaaatattgtactgcgcgcgactattataattgggacttcgaacactggggccggggtgccccggtcaccgtctcatcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 106 - DNA, 630 bp, synthetic construct
  misc_feature 1..630: note = Synthetic
  source 1..630: mol_type = other DNA
gaaattgtgttgacacagtctccagccaccctgtctttgtctccaggggaaacagccatcatctcttgtcggaccagtgagcatggtgacttagcctggtatcaacagaggcccggccaggcccccaggctcgtcatctattcgggttctactcgggccgctggcatcccagacaggttcagcggcagtcggtggggggcagactacaatctcagcatcagcaacctggagtcgggagattttggtgtttattattgtcagcagtatgaattttttggccaggggaccaaggtccaggtcgacatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 107 - DNA, 630 bp, synthetic construct
  misc_feature 1..630: note = Synthetic
  source 1..630: mol_type = other DNA
gaaattgtgttgacacagtctccagccaccctgtctttgtctccaggggaaacagccatcatctcttgtcggaccagtgagcatggtgacttagcctggtatcaacagaggcccggccaggcccccaggctcgtcatctattcgggttctactcgggccgctggcatcccagacaggttcagcggcagtcggtggggggcagactacaatctcagcatcaccaacctggagtcgggagattttggtgtttattattgtcagcagtatgaattttttggccaggggaccaaggtccaggtcgacatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 108 - DNA, 630 bp, synthetic construct
  misc_feature 1..630: note = Synthetic
  source 1..630: mol_type = other DNA
gaaattgtgttgacacagtctccagccaccctgtctttgtctccaggggaaacagccatcatctcttgtcggaccagtgagtatggtgacttagcctggtatcaacagaggcccggccaggcccccaggctcgtcatctattcgggttctactcgggccgctggcatcccagacaggttcagcggcagtcggtggggggcagactacaatctcagcatcagcaacctggagtcgggagattttggtgtttattattgtcagcagtatgaattttttggccaggggaccaaggtccaggtcgacatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 109 - DNA, 630 bp, synthetic construct
  misc_feature 1..630: note = Synthetic
  source 1..630: mol_type = other DNA
gaaattgtgttgacacagtctccagccaccctgtctttgtctccaggggaaacagccatcatctcttgtcggaccagtgatcatggttccttagcctggtatcaacagaggcccggccaggcccccaggctcgtcatctattcgggttctactcgggccgctggcatcccagacaggttcagcggcagtcggtggggggcagactacaatctcagcatcagcaacctggagtcgggagattttggtgtttattattgtcagcagtatgaattttttggccaggggaccaaggtccaggtcgacatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 110 - protein, 17 aa, synthetic construct
  REGION 1..17: note = Synthetic
WLKPRGGAVNYARKFQG

SEQ ID NO: 111 - protein, 126 aa, synthetic construct
  REGION 1..126: note = Synthetic
QVRLSQSGGQMKKPGESMRLSCRASGYEFLNCPINWIRLAPGRRPEWMGWLKPRGGAVNYARKFQGRVDMTRDVYSDTAFLELRSLTSDDTAVYFCTRGKYCTARDYYNWDFEHWGRGAPVTVSSA

SEQ ID NO: 112 - protein, 101 aa, synthetic construct
  REGION 1..101: note = Synthetic
EIVLTQSPATLSLSPGETAIISCRTSEHGDLAWYQQRPGQAPRLVIYSGSTRAAGIPDRFSGSRWGADYNLSISNLESGDFGVYYCQQYEFFGQGTKVQVD